(19) United States
(12) Patent Application Publication
(10) Pub. No.: US 2015/0141617 A1
(43) Pub. Date: May 21, 2015

(54) PRODUCTION AND PURIFICATION OF ACTIVE EUKARYOTIC FORMYLGLYCINEGENERATING ENZYME (FGE) VARIANTS

(71) Applicants: Universität Bielefeld, Bielefeld (DE); Georg-August-Universität Göttingen Stifung Öffentl, Goettingen (DE)

(72) Inventors: Thomas Dierks, Bielefeld (DE); Eva Charlotte Ennemann, Bielefeld (DE); Karthikeyan Radhakrishnan, Bielefeld (DE); Safaraz Alam, Goettingen (DE); Bernhard Schmidt, Goettingen (DE); Michaela Wachs, Bielefeld (DE)

(21) Appl. No.: 14/490,331

(22) Filed: Sep. 18, 2014

Related U.S. Application Data
(60) Provisional application No. 61/879,157, filed on Sep. 18, 2013.

(51) Int. Cl. C12N 9/02; C12P 21/00

(52) U.S. Cl. CPC C12N 9/0051; C12P 21/00; C12Y 108/99
USPC 530/350; 435/189; 435/68.1

(57) ABSTRACT

The invention features compositions and methods for generation and uses of formylglycine generating enzyme (FGE) variants.

This application claims priority benefit of U.S. provisional application Ser. No. 61/879,157, filed Sep. 18, 2013, which application is incorporated herein by reference in its entirety.

### FIELD

The present invention relates to the technical fields of cellular and molecular biology, biotechnology as well as medicine. The present invention relates to a procedure for the production of recombinant isolated polypeptides structurally based on the for C-α-formylglycine Generating Enzyme (FGE) having a genetically modified furin-cleavage motif and expression-systems for producing the same as well as methods and kits for use of the same.

Several documents are cited throughout the text of this specification. Each of the documents cited herein (including any manufacturer's specifications, instructions, etc.) are hereby incorporated herein by reference; however, there is no admission that any document cited is indeed prior art as to the present invention.

### BACKGROUND

Limited endoproteolysis of inactive precursor proteins at sites marked by paired or multiple basic amino acids is a widespread process by which biologically active peptides and proteins are produced within the secretory pathway in eukaryotic cells. However, many mammalian proteins prepared by genetic engineering technology are still accessible to posttranslational modification of enzyme, such as the family of endoproteases proteases, i.e. subtilisin/Kex2p-like proprotein convertases, which are conserved through bacteria, yeast as well as mammals wherein furin (EC 3.4.21.85) is the mammal homolog. The family has been shown to be responsible for conversion of precursors of peptide hormones, neuropeptides, and many other proteins into their biologically active forms. While in natural environment of the cell this is an important process in order to activate or regulate protein activity on a post-translational level, for processes for use in industrial mass production this is unwanted in case, where the proprotein is the target of the production.

Production of a prokaryotic FGE protein is described in U.S. Pat. No. 8,097,701 B2 or U.S. Pat. No. 8,349,910 B2, the disclosure content of these applications are herein incorporated by reference in its entirety. However, while the eukaryotic FGE enzyme differs inter alia by an N-terminal region encoded by eukaryotic-specific exon 1 compared to the prokaryotic FGE, this 55aa long N-terminal region (at least in human) can be post-translationally cleaved off within a eukaryotic cell, thus resulting in an N-terminal truncated (delta 72) eukaryotic FGE enzyme. This N-terminal truncated eukaryotic FGE is non-functional in vivo and requires the presence of strong reducing agents such as DTT to be active in vitro.

Formylglycine generating enzyme (FGE) post-translationally converts a specific cysteine in newly synthesized sulfatases to formylglycine (FGly). FGly is the key catalytic residue of the Sulfatase family, comprising 17 non-redundant enzymes in human that play essential roles in development and homeostasis. FGE, a resident protein of the endoplasmic reticulum, is also secreted. A major fraction of secreted FGE is N-terminally truncated lacking residues 34-72.

FGE is important for the generation of post-translational modifications on e.g. sulfatases. Sulfatases form a family of enzymes that catalyse the hydrolysis of sulfate esters and sulfamates in a wide variety of substrates like glycosaminoglycans, sulfolipids and steroid sulfates (1, 2). In pro- and eukaryotic sulfatases, post-translational modification of the crucial cysteine residue in the conserved C×P×R motif to formylglycine is a hallmark for their activation and sulfatases devoid of this modification are catalytically inactive. Multiple sulfatase deficiency (MSD), a rare but fatal lysosomal storage disorder in humans is characterized by the production of all 17 human sulfatases with almost no FGly formation in their active sites (3). The formylglycine-generating enzyme catalyzes this unique and critical modification in nascent sulfatase polypeptides in the endoplasmic reticulum (ER) and mutations in the FGE encoding gene SUMF1 were discovered as the basis of MSD (4-8). Recently, a role for FGE in regulating cell lineage commitment was reported. FGE, via activation of sulfatases Sulf1 and Sulf2, was shown to control haematopoietic lineage development through FGF and Wnt signalling (9).

In eukaryotes, FGE is localized in the lumen of the endoplasmic reticulum (ER). The mature 41-kDa protein in humans, lacking the signal peptide (aa 1-33), is a N-glycosylated monomer containing 8 cysteines. The core domain containing the active site exhibits a novel fold with remarkably low secondary structural elements that is stabilized by two Ca²⁺ ions and two intramolecular disulfide bridges (10). The presence of two catalytic cysteines (residues 336 and 341) in the active site, which are involved in binding and oxidation of the cysteine in the sulfatase polypeptide, is highly conserved in FGE homologues from prokaryotes to eukaryotes (11). One of the defining features unique to eukaryotic FGE is the presence of a 55-residue N-terminal extension (amino acid positions 34-88 in the mature form of the protein). The inventors have previously shown that this N-terminal extension of human FGE, for which the structure is unknown, is required for activation of sulfatases in cultured cells. Especially a conserved pair of cysteines (residues 50 and 52) within this extension was shown to be involved, with Cys52 being critical for this activation (12). Moreover, the N-terminal extension has been shown to confer efficient retention of FGE in the ER by interaction with ERp44, a redox sensor and retention factor for Ero1α and adiponectin (13, 14). FGE that escapes the ER-retention machinery is secreted (15). Recently, the re-uptake of secreted FGE has also been reported and interestingly, the endocytosed FGE has been shown to activate sulfatases after it reaches the ER by an unknown mechanism (16).

However, in previous studies the inventors have shown that a large fraction of secreted FGE is in an N-terminally truncated form starting at glutamate 73 (15). The nature of this proteolytic truncation and the identity of the protease(s) involved have not been defined so far. It is well known that proteolytic processing mediated by proprotein convertases (PCs) along the secretory pathway activates and thereby regulates the function of several secreted proteins (17). In case of FGE, however, the relevance of processing for controlling its function as a master regulator in sulfatase biogenesis has not been investigated so far and truncation of a functionally indispensable N-terminal fragment of FGE during secretion validates more detailed analysis of this process.

In U.S. Pat. No. 8,227,721 B2 the human FGE protein is co-expressed in a cell in order to activate sulfatases in vivo, the disclosure content of this application is herein incorporated by reference in its entirety. In contrast, the present invention uses the protein which is secreted into the cell culture medium for in vitro purposes.

In line with the above, there is a need for the provision of physiologically active eukaryotic full length (fl) FGE for use in the treatment of sulfatase related diseases wherein the FGE can be conveniently administered for the first time by way of conventional routes. In addition, enzyme productions of FGE using bacterial sources are well known in the art; see e.g. WO2012/097333.

However, the above mentioned mammalian as well as bacterial expression systems all suffer from the drawback that full length eukaryotic FGE cannot be stably produced since it will be rapidly degraded by proteases.

In addition, even though small amounts of eukaryotic full length FGE can be generated by a cell line, purification of the FGE protein will result in more than 90% cleaved FGE—also termed delta 72 FGE.

Furthermore, in order that delta 72 FGE does exhibit a certain activity in vitro, it therefore requires the presence of a strong reductant, such as β-mercaptoethanol or DTT. However, these reducing agents are likely to disrupt the endogenous disulfide bridges which are present in therapeutically active polypeptides such as antibodies. Correct pairing of disulfide bridges is mandatory for proper protein folding and thus the activity of therapeutically active polypeptides. As a result, for providing therapeutic grade of therapeutically active polypeptides the reduced, i.e. denatured polypeptides have to be correctly reoxidized/refolded which is in particular at a commercial scale generally time-, labor- and cost-consuming.

Moreover, therapeutically active antibodies are often coupled to drugs and used in therapy. For administering a drug, it is crucial to know how many drug molecules are coupled to the antibody in order to provide the correct amount of the drug (dosage). Thus, homogenously drug-coupled antibodies are highly appreciated in order to ensure correct treatment. Hence, there is a need for the provision of active full length (fl) eukaryotic FGE capable of generating aldehyde-formylglycine on a polypeptide in vitro using a mild/physiologically occurring reductant. In particular, there is a need for generating site-directed FGly-modifications of diagnostic proteins/peptides, therapeutic antibodies or medical polypeptides with the aim of downstream orthogonal aldehyde-mediated coupling reactions. Hence, there is a need for flFGE-encoding polynucleotide molecules and methods to achieve production of useful quantities of active fl FGE polypeptide.

The solution to said technical problem is achieved by providing the embodiments characterized in the claims, and described further below.

### SUMMARY

The amino acid sequence of eukaryotic full length formylglycine-generating enzyme (FGE) polypeptide was found to contain a unique sequence of amino acids containing a furin cleavage site (RYSR SEQ ID NO: 48 for the human polypeptide). Modifications of the polypeptide in order to inactivate the furin cleavage site, according to the present invention, provides modified protease resistant FGE polypeptides which are more stable when expressed in cell systems, including mammalian and insect cells, as compared with the unmodified FGE polypeptides.

It is therefore an object of the present invention to provide variants as well as processes for their production as well as FGE variant expressing cells in order to provide good expression yields in media substantially devoid of the types of foreign proteins and other impurities that could be problematic when the polypeptide is later used in the manufacture of products (e.g. manufacture of formylglycine-containing or aldehyde tags in pharmaceutical products). The FGE variants or fragments thereof differ from FGE wild type in their N-terminal furin-cleavage motif which is an exclusive feature of full length FGE.

The invention thus involves in one aspect an isolated FGE polypeptide, the cDNA encoding this polypeptide, functional modifications and variants of the foregoing, useful fragments of the foregoing, as well as diagnostics and therapeutics relating thereto.

It is a further object of the invention to provide a process for producing FGE-variants in insect cells which facilitate the expression of various eukaryotic FGE polypeptides in high amounts.

Furthermore, the present invention relates to a method for providing highly purified FGE polypeptide variants.

Another object of the invention is to provide FGE variants which can be used to generate an aldehyde tag on a therapeutically active polypeptide of interest wherein the reaction takes place under mild reducing conditions ex vivo, i.e. in vitro.

In a further embodiment the invention provides a polypeptide with an aldehyde tag produced by the FGE variants according to the present invention.

Another object of the present invention is the provision of a fragment of FGE variants essentially consisting of the amino acid sequence of the modified furin-cleavage motif for use as an inhibitor of furin or furin-like proteases or administered as a medicament.

These and other objects of the invention are achieved by providing FGE variants which exhibit a non-cleavable cleavage motif i.e. non-functional furin-cleavage motif at their N-terminal region, for which reason these variants can be expressed in high amounts as full length polypeptides and are thus capable of using glutathione as a reducing agent during their enzymatic reaction. The following aspects of the present disclosure are illustrative, but not inclusive of the full disclosure:
1. A process for producing eukaryotic Cα-formylglycine Generating Enzyme (FGE) or a functional variant thereof having Cα-formylglycine generating activity or a fragment thereof, comprising:
   (i) culturing an insect cell containing an isolated polynucleotide encoding the eukaryotic FGE enzyme or a functional variant or a fragment thereof under conditions permitting the expression of FGE or functional variant or a fragment thereof;
   (ii) obtaining the produced FGE polypeptide of step (i).
2. The process of aspect 1, wherein for the production of eukaryotic full length (fl) FGE₍₃₄₋₃₇₄ₐₐ₎, the polynucleotide encoding an eukaryotic fl FGE variant or a fragment thereof comprises a furin cleavage motif in the N-terminal region compared to the human fl FGE wild type (SEQ ID NO:2) which is non-cleavable, wherein the amino acid numbering of the fl FGE variant or fragment thereof corresponds to human FGE amino acid (SEQ ID NO:2)
3. The process of aspect 1, wherein, the insect cell stably express the isolated polynucleotide; or the process further comprises the following steps which are to be conducted prior to step (i) of aspect 1:
   (ia) infecting the cell with a recombinant baculovirus, wherein the virus containing an isolated polynucleotide encoding the eukaryotic FGE or a functional variant thereof or a fragment thereof;
   (ib) producing an infected insect cell capable of expressing FGE or a variant thereof
4. The process of aspect 2, wherein the furin cleavage motif having at least a core motif of the amino acid formula:

R-Y-S-R
corresponding to human FGE amino acid (SEQ ID NO:2) aa 69-72.
5. The process of aspect 3, wherein the baculovirus is Autographa californica multicapsid nucleo polyhedrovirus (AcMNPV) or Bombyx mori nuclear polyhedrovirus (BmNPV).
6. The process of aspect 1, wherein the insect cell is selected from the group essentially consisting of cells derived from Spodopterafrugiperda, Trichoplusiani, Plutellasylostella, Manducasextra and Mamestrabrassicae; preferably wherein the insect cell is selected from the group consisting of Schneider cells S2 and S3, SF9, SF21, High FiveCells (BTI-TN-5B1-4), D.Mel-2 cells KCl cells and Mimi Sf9 insect cells.
7. The process of aspect 1, wherein the eukaryotic FGE species is selected from the group consisting of mammalian, human, fungus, algae and insect.
8. The process of aspect 1, wherein the species is human.
9. A Eukaryotic Cα-formylglycine Generating Enzyme (FGE) or a functional variant thereof having Cα-formylglycine generating activity or a FGE fragment obtainable by the process of aspect 1, wherein the obtained eukaryotic polypeptide exhibit insect-specific post-translational modifications.
10. A eukaryotic FGE polypeptide variant having Cα-formylglycine generating activity, wherein said variant comprises an amino acid sequence comprising a furin cleavage motif wherein the furin core cleavage motif having at least a core motif of the amino acid formula R-Y-S-R corresponding to human FGE amino acid (SEQ ID NO:2) aa 69-72 and having one or more amino acid modifications in the furin-cleavage motif, wherein the exchange results in
   i) an FGE variant having a non-cleavable furin cleavage motif; or
   ii) an FGE variant having an optimized furin cleavage motif, wherein the one or more of the amino acid modifications is located in the furin core cleavage motif, and/or
   the one or more amino acid modifications takes place in the extended furin-cleavage motif comprising:

Xₙ₋₆-R-Y-S-R-Xₙ₊₈,
corresponding to human FGE amino acid (SEQ ID NO: 2) aa 63-80, wherein
(iii) Xₙ₋₆ is SSAAAH in position 63 to 68,
(iv) Xₙ₊₈ is EANAPGPV in position 73 to 80,
wherein at least one amino acid residue in (i) to (iv) is changed compared to the wild type.
11. The polypeptide of aspect 10, wherein the polypeptide exhibits at least one of the following characteristics:
   (a) is at least a 41 kDa+/−3 kDa protein (SDS-PAGE) and/or has a 55 aa N-terminal extension compared to a prokaryotic FGE protein
   (b) exhibits in vitro formlyglycine generation activity;
   (c) is stable during chromatographic purification process;
   (d) exhibits the N-terminal sequence EAN (Glu-Ala-Asn);
   (e) exhibits an amino acid sequence having 85% or more identity to human FGE amino acid sequence (SEQ ID NO: 2); and/or;
   (f) catalyzes thiol-to-aldehyde oxidation of cysteine residues in the presence of glutathione.
12. The polypeptide of aspect 10, wherein
   i) the variant comprises at least one of the substitutions selected from the group consisting of SEQ ID NO:8, SEQ ID NO: 10, SEQ ID NO:12, SEQ ID NO: 14, SEQ ID NO: 16, SEQ ID NO: 18, SEQ ID NO:20; (SEQ ID NO:22, SEQ ID NO:24; SEQ ID NO:26, SEQ ID NO:4, SEQ ID NO:29 and SEQ ID NO: 31, or a combination thereof; and
   ii) wherein the amino acid sequence of the variant comprises of an amino acid sequence having at least a degree of identity to SEQ ID NO: 2 of at least 60%, such as at least 65%, 70%, 80%, 85% or 90%.
13. An isolated polynucleotide derived from a eukaryotic organism, comprising a nucleic acid sequence that code for the polypeptide of aspect 12.
14. A method of inhibiting a furin or furin-like protease comprising (i) contacting a target with the polypeptide of aspect 10, wherein the polypeptide consisting of the amino acid of the non-functional furin cleavage motif of aspect 10 (i).
15. A primer, comprising one of the following sequences (5′->3′)

(SEQ ID NO: 7)
TCGGCAGCCGCTCACGCATACTCGCGGGAGGCT
(SEQ ID NO: 9)
TCGGCAGCCGCTCACAAATACTCGCGGGAGGCT
(SEQ ID NO: 11)
GCAGCCGCTCACCGAGCCTCGCGGGAGGCTAAC
(SEQ ID NO: 13)
GCAGCCGCTCACCGAAAGTCGCGGGAGGCTAAC
(SEQ ID NO: 15)
GCAGCCGCTCACCGATTCTCGCGGGAGGCTAAC
(SEQ ID NO: 17)
GCAGCCGCTCACCGATCCTCGCGGGAGGCTAAC,
(SEQ ID NO: 19)
GCCGCTCACCGATACGCGCGGGAGGCTAACGCT
(SEQ ID NO: 21)
GCCGCTCACCGATACAGGCGGGAGGCTAACGCT
(SEQ ID NO: 23)
GCTCACCGATACTCGAAGGAGGCTAACGCTCCG
(SEQ ID NO: 25)
GCTCACCGATACTCGGCGGAGGCTAACGCTCCG
(SEQ ID NO: 27)
GCTCACGCATACTCGGCGGAGGCTAACGCTCCG
(SEQ ID NO: 28)
GCCGCTCACCGAGCCAGGCGGGAGGCTAACGCT
(SEQ ID NO: 30)
CACCGATACTCGCGGCCGGCTAACGCTCCGGGC
(SEQ ID NO: 32)
CCGGAATTCAGCCAGGAGGCCGGGACC

16. A vector comprising the polynucleotide of aspect 12 or a functional fragment thereof
17. The vector of aspect 16, wherein the nucleic acid further encodes a purification tag and/or linker.
18. A host cell expressing the vector of aspect 16 or 17.
19. The process of aspect 1, wherein the vector of aspect 16, the host cell of aspect 18 or a suitable primer of aspect 15 is used in the process of aspect 1.
20. The host cell of aspect 18, wherein the cell is selected from the group consisting of mammalian, human, algae, fungus and insect cell.
21. An antibody which selectively binds to the FGE variant of aspect 10.
22. The process of aspect 1 or the cell according to aspect 20, wherein the produced FGE polypeptide is secreted into the medium.
23. A method of providing purified FGE or a functional variant thereof, the method comprising the steps of
   i) (i) to (ii) of the process of aspect 1, wherein the vector of aspect 16 or 17 is expressed;
   ii) collecting the produced FGE enzyme or variant from the cell culture medium; and
   iii) purifying the produced FGE enzyme or variant thereof by chromatographic means.
24. An in vitro method of producing a tag in a polypeptide of interest, comprising the steps of
   (i) incubating a polypeptide having a motif comprising a sulfatase motif having a 2-formylglycine, together with the FGE polypeptide or a functional variant thereof of aspect 9 or aspect 10 in the presence of a reducing agent under conditions suitable for enzymatic activity to allow conversion of an amino acid residue to a formylglycine (FGIy) residue in the polypeptide and produces a converted tagged polypeptide;
   (ii) recovering the polypeptide with the newly generated tag.
25. The method of aspect 24, further comprising the step of
   (iii) attaching a moiety of interest to the newly generated tag, wherein the moiety is selected from the group consisting of detectable label, a small molecule, a peptide or a toxin.
26. The method of aspect 24, wherein glutathione is used as a reducing agent.
27. The method of aspect 24, wherein the tag is an aldehyde tag.
28. The method of any aspect 25, wherein the polypeptide is a medicament or a vaccine.
29. A polypeptide with a tag obtained by the method of aspect 24 or the polypeptide with a tag, which further comprises a moiety obtained by the method of aspect 25.
30. A composition comprising the FGE polypeptide or variant thereof of aspect 9 or obtained by the method of aspect 23 or the highly purified FGE polypeptide or variant obtained by the method of aspect 24 or the tagged polypeptide generated by the method of aspects 24 or 25.
31. The composition of aspect 30, which is
   (i) a pharmaceutical composition and further comprises a pharmaceutically acceptable carrier, or
   (ii) a diagnostic composition and optionally comprising reagents conventionally used in immuno or nucleic acid based diagnostic methods.
32. A method of treating or diagnosing a subject suffering from Multiple Sulfatase Deficiency (MSD) or a FGE deficiency related disease or condition comprising administering an therapeutically or diagnostically effective amount of the composition of aspect 30 comprising a fl FGE variant and a pharmaceutical acceptable carrier to the subject.
33. A kit comprising the polypeptide of aspect 9 or the cell of aspect 18 or the polypeptide of aspect 12, or at least one primer of aspect 15, the vector of aspect 16 or 17, optionally with reagents and/or instructions for use.
34. The kit of aspect 33, further comprising imidazole.
35. The process of aspect 1, wherein the process produces biologically active fl FGE variant (SEQ ID NO: 2, SEQ ID NO: 4, SEQ ID NO: 8 or SEQ ID NO: 26).
36. The process of aspect 35, wherein the fl FGE variant is produced in dimeric form.
37. The process of aspect 24, wherein the produced polypeptide is a non-naturally occurring or modified non-naturally occurring, recombinant polypeptide.
38. The process of aspect 37, wherein the modified non-naturally occurring, recombinant polypeptide comprising a heterologous sulfatase motif having a 2-formylglycine residue covalently attached to a moiety of interest.
39. The process of aspect 38, wherein the modified non-naturally occurring, recombinant polypeptide is selected from the group consisting of an Fc fragment, an antibody, an antigen-binding fragment of an antibody, a blood factor, a fibroblast growth factor, a protein vaccine, and an enzyme.

These and other objects are provided by the inventions disclosed herein.

### BRIEF DESCRIPTION OF THE DRAWINGS

FIG. 1: Analysis of the fl-FGE (WT) expression. High Five cells grown in suspension were infected with indicated volumes of fl-FGE-WT recombinant virus stock (2nd generation). 25 μL of cell lysate and 25 μl of medium were resolved by SDS-PAGE followed by detection of FGE after western blotting using FGE-antiserum.

FIG. 2: Analysis of the fl-FGE-R69A/R72A expression. High Five cells grown in suspension were infected with different volumes of fl-FGE-R69A/R72A recombinant virus stock (2nd generation). FGE expression was analyzed by separating 100 μL of culture supernatants by SDS-PAGE followed by detection of FGE after western blotting using FGE-antiserum. Δ72-FGE (lane 8) was loaded for comparison.

FIG. 3: Analysis of the Ni-NTA purification of fl-FGE-R69A/R72A from insect cell expression supernatant. 230 mL of conditioned medium were subjected to Ni-NTA affinity purification as described in the text. Aliquots of each fraction were separated by SDS-PAGE and visualized by coomassie-staining. The amount loaded (in μL and % of the whole fraction), protein concentrations determined by Bradford assay and calculated total amounts are shown.

FIG. 4: Analysis of the Δ72-FGE-wt expression. High Five cells grown in suspension were infected with different volumes of Δ72-FGE-wt recombinant virus stock (2nd generation). FGE expression was analyzed by separating 100 μL of culture cells (C) and supernatants (M) by SDS-PAGE followed by detection of FGE either by western blotting using FGE-antiserum or by coomassie staining.

FIG. 5: Purification of Δ72-FGE by His-Trap affinity chromatography. 400 ml of conditioned express five media were subjected to His Trap affinity purification as described earlier in the text. The elution fractions after affinity purification were analyzed by SDS-PAGE and visualized by coomassie-staining. 50 μl of starting material (Load) and flow through (FT) and 20 μl of elution fractions (2%) were taken out from each fraction and were analyzed. Fractions containing Δ72-FGE that resolves at 37 kDa were pooled and the indicated protein concentrations were determined by Bradford assay.

FIG. 6: Identification of FGE by LC-MALDI MS/MS. The amino acid sequence of human FGE is shown. Using LC-MALDI MS/MS and database search in the NCB Inr database, tryptic FGE peptides (marked in red) could be identified with E-values between 2.2e⁻³ and 3.3e⁻¹². This constitutes sequence coverage of 62%. Large (>3000 Da) or very short peptides (<500 Da) were out of mass range and were not identified.

FIG. 7: MALDI-ToF MS analysis of the 23-aa peptide. A representative mass spectrum of MALDI-ToF MS analysis of the 23-aa peptide showing both, the cysteine containing substrate peptide (2526.3 m/z) and the FGly-containing product peptide (2508.3 m/z), modified by fl-FGE-R69A/R72A from HighFive cells.

FIG. 8: FGE is N-terminally truncated in a post-ER compartment. A, FGE is N-terminally processed only upon secretion. HT1080 Tet-On cells were transiently transfected with cDNA encoding FGE-HA or FGE with an appended KDEL signal (FGE-HA-KDEL). 6 h post-transfection, FGE expression was induced with 20 ng/mL doxycycline. After 22 h of induction, cells (C) and medium (M) were analyzed by western blotting with FGE antiserum. B, FGE truncation is observed in various cell lines. FGE was transiently expressed in the indicated cell lines for 24 h and cells and medium were analyzed by western blotting with FGE antiserum. C, N-terminal processing of FGE is independent of its expression level. FGE was transiently expressed in HT1080 Tet-On cells and expression was induced with the indicated concentrations of doxycycline. After 22 h of induction, cells and medium (in a ratio of 2:1) were analyzed by western blotting. The amount of FGE in cells and medium was determined by calibration of the western blot with known amounts of purified FGE (not shown). D, Endogenous FGE is also secreted and proteolytically processed. HT1080 cells were cultured for 48 h and cell lysates and conditioned media were subjected to immunoprecipitation (IP) with FGE antiserum or preimmune serum (PIS). 100% of IP-fractions were analyzed by western blotting. Non-conditioned (non-c.) cell culture medium served as a control.

FIG. 9: The RXXR motif is required for proteolytic processing of secreted FGE but not for activity. A, Schematic representation of human FGE with the RYSR motif at the cleavage site (arrow). The cysteine residues are highlighted as black lines and the calculated molecular masses of fl- and processed Δ72-FGE are indicated. SP, signal peptide. B,HT1080 Tet-On cells were transiently transfected with FGE wildtype (Wt) and the indicated alanine variants of the FGE-RYSR-motif. 6 h post-transfection, FGE expression was induced with 20 ng/mL doxycycline. After induction for 20 h, cells (C) and medium (M) at a ratio of 2:1 were analyzed for FGE by western blotting using FGE anti-serum. The amount of FGE in the cells and medium was determined by calibration of the western blot with known amounts of purified FGE protein.C, MSDi Tet-On cells were transiently transfected with steroid sulfatase (STS) and FGE-wt or FGE-RYSR-motif variants in the indicated combinations. The amounts of STS and FGE were monitored in the cell extracts by western blotting. The relative specific activity of STS given below the lanes was calculated from the STS activity (nmol/h per mg cell protein) divided by the western blot signal of STS (arbitrary units/mg cell protein) and referred to that in cells expressing STS only.

FIG. 10: The RYSR motif is conserved in later diverging eukaryotes. The phylogenetic tree (left) of 13 representative species, chosen out of a total of 88 analyzed species (see Table 2), was generated from Newick format according to modern molecular consensus taxonomy (27) and visualized with Phylodendron (http://iubio.bio.indiana.edu/treeapp/treeprint-form.html). The species were divided into three groups based on their taxonomy and the presence of RYSR or YS at the cleavage site position. Group I represents 36 species from euarchontoglires, laurasiatheres and atlantogenata, group II consists of 22 sequences from marsupials to ray-finned fish and group III includes 30 species of urochordates to basal metazoan. WebLogo 3.0 was used to create Logos (I-III) of the three groups as well as a combined Logo (IV) of all 58 later diverging eukaryote sequences (19, 20). The four sequence logos display the degree of conservation of amino acids at positions P8 to P8′ of each group (representing residues 65-80 of human FGE) and were generated as described in Experimental Procedures. A high degree of conservation of a single amino acid at a particular position is represented by a large size (in units of bits) of the amino acid letter in the logo. Colors represent chemical properties (polar, basic, acidic, hydrophobic). Cleavage takes place between P1 and P1′, marked by arrows.

FIG. 11: Analysis of the conserved RYSR↓E cleavage motif by alanine scanning mutagenesis. A, B, HT1080 Tet-On cells were transiently transfected with pBI plasmids encoding FGE-wt (with RYSR↓E motif) or mutants thereof (with the mutated residues in the RYSR↓E motif indicated in bold). FGE expression was induced with 20 ng/mL doxycycline, 6 h after transfection. After 20 h of induction, cells and medium (at a ratio of 2:1) were analyzed by western blotting with FGE antiserum (upper panels). The cleavage efficiency was quantified from these blots and expressed as signal ratio of fl-FGE/Δ72-FGE in the medium, as indicated below each medium (M) lane in a bar graph. These data are representative of two independent experiments.

FIG. 12: Proteolytic processing of FGE is mediated by furin. A, Processing of secreted FGE is inhibited by the PC inhibitor RVKR-CMK. HT1080 cells transiently expressing FGE were treated with the indicated concentrations of the inhibitor. After 16 h of treatment, cells and medium were analyzed by western blotting. B, Impaired processing of secreted FGE in furin-deficient LoVo cells. Cells and medium from HT1080 and LoVo cells stably expressing FGE were analyzed by western blotting. C,FGE processing is abolished in furin-deficient CHO cells (CHO-FD11) but efficiently restored by co-expression of furin. Wild type CHO (CHO-K1) and CHO-FD11 cells transiently expressing FGE or CHO-FD11 cells transiently co-expressing furin and FGE were cultured for 24 h. Cells and media were analyzed by western blotting. D, Cellular and secreted FGE are processed by recombinant furin (rFurin) in vitro. Cell lysate and medium of CHO-FD11 cells stably expressing FGE were incubated at 25° C. for 3 h either in presence or absence of rFurin. E, Endogenous cellular FGE from HT1080 cells is cleaved by rFurin in vitro and this processing is inhibited by the RVKR-CMK-inhibitor. Equal amounts of HT1080 cell lysate were incubated with rFurin and 25 μM CMK-inhibitor as indicated. A-E, All western blots were probed with FGE antiserum.

FIG. 13: Proteolytic processing of FGE by other furin-like proteases and extracellular processing of secreted FGE. A, Cells and medium of CHO-FD11 Tet-On cells transiently expressing FGE alone or coexpressing the PCs furin, PACE4, PCSa or PC7 for 24 h were analyzed by western blotting using FGE antiserum (upper panel). The PC expression level was indirectly determined by analysis of the cell lysates for expression of EGFP, driven from the downstream IRES element (see Experimental Procedures), using an anti-GFP antibody (lower panel). B, Conditioned medium from CHO-FD11 cells stably expressing FGE was added to MSDi, HeLa, HEK293, CHO-FD11 or HT1080 cells, or left untreated (control) and incubated for 20 h before being analyzed by western blotting using FGE antiserum. C, Conditioned medium from CHO-FD11 cells was incubated with HEK293 cells for the indicated time points and analyzed as above.

FIG. 14: Furin-mediated processing of FGE leads to inactivation. FGly-generating activity was measured in vitro using conditioned media from CHO-FD11 cells containing fl-FGE, or from CHO-FD11 cells containing Δ72-FGE due to co-expression of FGE and furin. The activity assay was performed in triplicates with three sets of conditioned media as described in Experimental Procedures. A, Representative spectra of MALDI-ToF mass spectrometry analysis of the substrate peptide after incubation with FGE (for 20 min; a, b) or Δ72-FGE (for 30 min; c, d) containing conditioned medium using either 2 mM dithiothreitol (DTT) or 5 mM glutathione (GSH) as reducing agents, as indicated. The cysteine substrate peptide is detected showing a monoisotopic mass at 2526.3 m/z (a-d), while the corresponding signal of the FGly-containing product peptide appears at 2508.3 m/z (a-c), as indicated. B, The bar graph displays relative substrate peptide turnover with GSH or DTT as reductant for fl-FGE (a, b) and Δ72-FGE (c, d); substrate turnover in the presence of GSH is normalized to that of the corresponding DTT sample (100%). Mean values of triplicates of one representative experiment are shown.

FIG. 15: FGE in complex with ERp44 resists furin cleavage. Equal amounts of NEM-treated HT1080 Tet-On cell lysates (lysed without protease inhibitor) expressing either FGE alone or coexpressing FGE and myc-ERp44 were subjected to in vitro furin cleavage (see Experimental Procedures). Samples were boiled in SDS-PAGE sample buffer (with or without β-mercaptoethanol), subjected to SDS-PAGE under non-reducing (−SH, upper panel) or reducing (+SH, lower panel) conditions and analyzed by western blotting using either anti-FGE or anti-myc antibodies or both, as indicated. Note that FGE processing by endogenous furin (as indicated by the appearance of band * in lane 2) is abolished in the presence of protease inhibitor (lane 1).

FIG. 16: Separation of the recombinant fl-FGE-R69A/R72A monomer and dimer by size-exclusion chromatography. (A) The pooled elution fractions from Ni-NTA purification (SM) were further purified in a Superdex (SD200 10/300 GL) column using Ettan LC system (GE health care). The eluted material in 0.5 ml of running buffer (20 mM Tris, pH 8.0 containing 200 mM NaCl) were collected and analyzed in SDS-PAGE under reducing conditions followed by coomassie staining (B) Equal volume of elution fractions obtained as shown in ‘(A)’ were resolved in SDS-PAGE under non-reducing conditions followed by western blot and membrane decorated with FGE anti-serum. (SM: starting material; FGE: monomer; FGE2: dimer).

FIG. 17: Stability of purified fl-FGE-R69A/R72A. (A) Recombinant fl-FGE-R69A/R72A after Ni-NTA purification (panel Ni-NTA) which was stored at −80° C. for 4 weeks and the monomer (M) and dimer (D) of recombinant fl-FGE-R69A/R72A after Size exclusion chromatography (panel SEC) were analyzed by SDS-PAGE followed by Coomassie staining. (B) 250 mM imidazole was added to aliquots of the purified fraction from size exclusion chromatography, stored at −80° C. for two weeks and subsequently analysed by SDS-PAGE and Coomassie staining Using 2D-densitometry, the total amount of FGE (fl-form+truncated forms) was calculated from which the amount of FGE in the fl-form (shown as % fl-FGE) was deduced.

FIG. 18: Dependence of fl-FGE-R69A/R72A activity on DTT and GSH. (A) 13 ng of either monomeric or dimeric FGE was incubated with the substrate peptide (16 pmol) under standard assay conditions (see text) for 15 min at 37° C. in the presence of up to 15 mM DTT. After addition of 2 μl 10% TFA to stop the reaction, MALDI-TOF mass spectrometry (Ultraflex 2, Bruker) was used to determine the ratio of product to substrate peptide from which the percentage of substrate turnover was calculated. Data points shown are the mean±S.E. of triplicates. (B) 18 ng of either monomeric or dimeric FGE was incubated with the substrate peptide (16 pmol) under standard assay conditions (see text) for 20 min at 37° C. in the presence of up to 15 mM GSH. The reaction was stopped by addition of 2 μl 10% TFA and activity (expressed as percent substrate turnover) measured as shown for DTT-dependent assays. Data points shown are the mean of duplicates.

FIG. 19: Dependence of fl-FGE-R69A/R72A activity on pH. For analyzing the pH-dependence of FGE activity, the reaction was carried out under standard assay conditions in MTGC buffer (50 mM MOPS, 50 mM Tris, 50 mM Glycine and 50 mM CAPS) with pH in the range of 6.5-11.0. (A) 13 ng of either monomeric or dimeric FGE was incubated with the substrate peptide (16 pmol) under standard assay conditions in MTGC buffer of indicated pH for 20 min at 37° C. in the presence of 2 mM DTT. After addition of 2 μl 10% TFA to stop the reaction, MALDI-TOF mass spectrometry (Ultraflex 2, Bruker) was used to determine the ratio of product to substrate peptide. Data points shown are the mean of duplicates or the mean±S.E. of triplicates (for pH 9-10). (B) 28 ng of monomeric FGE or 28 ng of dimeric FGE was incubated with the substrate peptide (16 pmol) under standard assay conditions in MTGC buffer of indicated pH for 20 min at 37° C. in the presence of 5 mM GSH. The reaction was stopped by addition of 2 μl 10% TFA and activity measured as shown for DTT-dependent assays. Data points shown are the mean of duplicates or the mean±S.E. of triplicates (for pH 9-10).

FIG. 20: Dependence of Δ72-FGE activity on pH. (A) FGE (4 ng) was incubated with the substrate peptide (16 pmol) under standard assay conditions in MTGC buffer of indicated pH for 20 min at 37° C. in the presence of 2 mM DTT. After addition of 2 μl 10% TFA to stop the reaction, MALDI-TOF mass spectrometry (Ultraflex 2, Bruker) was used to determine the ratio of product to substrate peptide. Data points shown are the mean±S.E. of triplicates.

### SEQUENCE LISTING

The Sequence Listing associated with this application is filed in electronic form via EFS-Web and is hereby incorporated by reference into this specification in its entirety.

### DEFINITIONS

Unless otherwise stated, a term as used herein is given the definition as provided in the Oxford dictionary of biochemistry and molecular biology, Oxford University Press, 1997, revised 2000 and reprinted 2003, ISBN 0 19 850673 2.

For further elaboration of general techniques useful in the practice of this invention, the practitioner can refer to standard textbooks and reviews in cell biology and tissue culture; see also the references cited in the examples. General methods in molecular and cellular biochemistry can be found in such standard textbooks as Molecular Cloning: A Laboratory Manual, 3^{rd} Ed. (Sambrook et al., Harbor Laboratory Press 2001); Short Protocols in Molecular Biology, 4th Ed. (Ausubel et al. eds., John Wiley & Sons 1999); Protein Methods (Bollag et al., John Wiley & Sons 1996); Non-viral Vectors for Gene Therapy (Wagner et al. eds., Academic Press 1999); Viral Vectors (Kaplitt & Loewy eds., Academic Press 1995); Immunology Methods Manual (Lefkovits ed., Academic Press 1997); and Cell and Tissue Culture: Laboratory Procedures in Biotechnology (Doyle & Griffiths, John Wiley & Sons 1998). Reagents, cloning vectors and kits for genetic manipulation referred to in this disclosure are available from commercial vendors such as BioRad, Stratagene, Invitrogen, Sigma-Aldrich, and ClonTech.

“Amino acid” refers to any of the twenty standard α-amino acids as well as any naturally occurring and synthetic derivatives. Modifications to amino acids or amino acid sequences can occur during natural processes such as posttranslational processing, or can include known chemical modifications. Modifications include, but are not limited to: formylglycine, phosphorylation, ubiquitination, acetylation, amidation, glycosylation, covalent attachment of flavin, ADP-ribosylation, cross linking, iodination, methylation, and the like.

As used herein, the term “C[alpha]-formylglycine generating activity” refers to the ability of a molecule to form, or enhance the formation of, FGly on a substrate. The substrate may be a sulfatase as described elsewhere herein e.g. EP 2 235 301 A1, a synthetic oligopeptide (see, e.g., SEQ ID NO: 46, and the Examples), a recognition sequence as used in WO2009/120611 and/or WO2012/097333 A2. The disclosure content of these applications is herein incorporated by reference in its entirety. The substrate preferably contains the conserved hexapeptide of SEQ ID No:47 [L/V-C-X-P-S-R] or any of the modified sequences mentioned in WO2009/120611 and/or WO2012/097333 A2. Methods for assaying FGly formation are described in the art (see, e.g., Dierks, T., et al., Proc. Natl. Acad. Sci. U.S.A., 1997, 94:11963-11968), and elsewhere herein (see, e.g., the Examples).

The enzyme that oxidizes cysteine in a sulfatase motif to FGly is referred to herein as a formylglycine generating enzyme (FGE). As discussed above, unless otherwise indicated the term “FGE” is used herein to refer to FGly-generating polypeptides that mediate conversion of a cysteine (C) of a sulfatase motif to FGly.

In general, an FGE for use in the methods disclosed herein can be obtained from naturally occurring sources or synthetically produced. For example, an appropriate FGE can be derived from biological sources which naturally produce an FGE or which are genetically modified to express a recombinant gene encoding an FGE. Nucleic acids encoding a number of FGEs are known in the art and readily available (see, e.g., Preusser et al. 2005 J. Biol. Chem. 280(15): 14900-10 (Epub 2005 Jan. 18); Fang et al. 2004 J Biol Chem. 79(15): 14570-8 (Epub 2004 Jan. 28); Landgrebe et al. Gene. 2003 Oct. 16; 316:47-56; Dierks et al. 1998 FEBS Lett. 423(1):61-5; Dierks et al. Cell. 2003 May 16; 113(4):435-44; Cosma et al. (2003 May 16) Cell 113(4):445-56.

Dierks et al. Cell. 2005 May 20; 121(4):541-52; Roeser et al. (2006 Jan. 3) Proc Natl Acad Sci USA 103(1):81-6; Sardiello et al. (2005 Nov. 1) Hum Mol Genet. 14(21):3203-17; WO 2004/072275; WO 2008/036350; U.S. Patent Publication No. 2008/0187956; and GenBank Accession No. NM_{—}182760. Accordingly, the disclosure here provides for recombinant host cells genetically modified to express an FGE polypeptide/variant that is compatible to produce an aldehyde tag of a tagged target polypeptide and/or to produce formylglycine (FGly). In certain embodiments, the FGE used may be a naturally occurring polypeptide and/or enzyme (may have a wild type amino acid sequence). In other embodiments, the FGE used may be non-naturally occurring, in which case it may, in certain cases, have an amino acid sequence that is at least 80% identical, at least 90% identical or at least 95% identical to that of a wild type enzyme. Because FGEs have been studied structurally and functionally and the amino acid sequences of several examples of such enzymes are available, variants that retain enzymatic activity should be readily designable. FGE as defined above relates to the wild type FGE enzyme.

As used herein, the terms “wild type”, “wt”, “wild-type (wt) FGE polynucleotide,” “wild-type FGE DNA,” and “wild-type FGE (poly)nucleic acid” refer to SEQ ID NO: 1. SEQ ID NO: 2 is the mature peptide sequence (i.e., containing no signal peptide) of FGE that is endogenously expressed by a human cell. In one embodiment, the term wild type includes the FGE polypeptide sequence without the signal peptide and leader peptide i.e. without aa 1-33.

The terms “polypeptide”, “peptide”, “enzyme”, and “protein” are used interchangeably herein to refer to a polymer of amino acid residues. The terms apply to amino acid polymers in which one or more amino acid residue is an analogue of a corresponding naturally occurring amino acid, as well as to naturally occurring amino acid polymers. The term also includes variants on the traditional peptide linkage joining the amino acids making up the polypeptide.

As used herein, the term “variant”, “functional variant” refers to a FGE polypeptide or polynucleotide encoding a FGE polypeptide comprising one or more modifications relative to wild-type (wt) FGE polypeptide or the wild-type polynucleotide encoding FGE (such as substitutions, insertions, deletions, and/or truncations of one or more amino acid residues or of one or more specific nucleotides or codons in the polypeptide or polynucleotide, respectively). A “variant” or “modified FGE polypeptide” are used herein interchangeably and include polypeptides having an amino acid sequence sufficiently similar, i.e. have an amino acid sequence that is at least 80% identical, at least 90% identical or at least 95% identical to that of a wild type enzyme to the amino acid sequence of the natural FGE full length polypeptide, i.e. at least to the amino acid sequence of 73 to 374, at least to 69 to 374, at least 63 to 374, at least 34 to 374 amino acids of the human FGE amino acid sequence of SEQ ID NO: 2.

As used herein, the terms “numbered with reference to”, “compared to” or “corresponding to,” when used in the context of the numbering of a given amino acid or polynucleotide sequence, refers to the numbering of the residues of a specified reference sequence when the given amino acid or polynucleotide sequence is compared to the reference sequence. The following nomenclature may be used to describe substitutions in a test sequence relative to a reference sequence polypeptide or nucleic acid sequence: “R-#-V,” where # refers to the position in the reference sequence, R refers to the amino acid (or base) at that position in the reference sequence, and V refers to the amino acid (or base) at that position in the test sequence, in some embodiments, an amino acid (or base) may be called “X,” by which is meant any amino acid (or base). As a non-limiting example, for a variant polypeptide described with reference to a wild-type FGE polypeptide (e.g., SEQ ID NO: 2), “R69A” indicates that in the polypeptide being compared, the R at position 69 of the reference sequence is replaced by A, with amino acid position being determined by optimal alignment of the variant sequence with SEQ ID NO:2.

For the purposes of the present invention, the term “substantially similar” or “sufficiently similar” or “similar” means a first amino acid sequence that contains a sufficient or minimum number of identical or equivalent amino acid residues relative to a second amino acid sequence such that the first and second amino acid sequences have a common structural domain and/or common functional activity. For example, amino acid sequences that comprise a common structural domain that is at least about 45%, at least about 50%, at least about 55%, at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, at least about 99%, or at least about 100%, identical are defined herein as sufficiently similar. Amino acid substitutions which are conservative substitutions unlikely to affect biological activity are considered identical for the purposes of this invention and include the following: Ala for Ser, Val for Ile, Asp for Glu, Thr for Ser, Ala for Gly, Ala for Thr, Ser for Asn, Ala for Val, Ser for Gly, Tyr for Phe, Ala for Pro, Lys for Arg, Asp for Asn, Leu for Ile, Leu for Val, Ala for Glu, Asp for Gly, and the reverse. (See, for example, Neurath et al., The Proteins, Academic Press, New York (1979)). Further information regarding phenotypically silent amino acid exchanges can be found in Bowie et al., 1999, Science 247:1306-1310).

The term “fragment thereof” generally denotes a truncated, i.e. shorter version of the FGE enzyme or FGE variant as defined above.

The term a “fragment of FGE variant”, “functional variant” or “FGE variant or a fragment thereof” are used interchangeably herein and relates to a peptide i.e. amino acid sequence as defined above comprising at least the furin core (SEQ ID NO: 48), preferably also the furin cleavage motif (SEQ ID NO:45), thus consists of at least 3, 4, 5, 6, 7, 8, 9, 10, 11, 12 13, 14 15, 16, 17, 18, 19 or 20 amino acids corresponding to the amino acid sequence in position 63 to 80 of the human FGE enzyme (SEQ ID NO: 2). Preferably, the variant fragment is biologically active fragment. As used herein, the term “biologically active fragment,” refers to a polypeptide that has an amino-terminal and/or carboxy-terminal deletion(s) and/or internal deletion(s), but where the remaining amino acid sequence is identical to the corresponding positions apart from the furin cleavage motif (aa 63 to 80) in the sequence to which it is being compared (e.g. a full-length human FGE of the present invention) and that retains substantially all of the activity of the full-length FGE biologically active fragment can comprise about 60%, about 65%, about 70%, about 75%, about 80%, about 85%, at about 90%, about 91%, about 92%, about 93%, about 94%, about 95%, about 96%, about 97%, about 98%, or about 99% of a full-length FGE polypeptide.

The term “FGE or fragment thereof”, “fragment of FGE polypeptide” or “fragment of FGE enzyme” are used interchangeably herein and relates to a polypeptide of at least 18 amino acids produced by the insect cells of the invention can also relate to any amino acid fragment of FGE enzyme such as the N-terminally truncated FGE enzyme consisting of amino acid sequence 72 to 374, or only a C-terminal domain or a genetically engineered hybrid consisting of different FGE domains as long as the fragment is produced by the insect cells. A fragment can comprise about 60%, about 65%, about 70%, about 75%, about 80%, about 85%, at about 90%, about 91%, about 92%, about 93%, about 94%, about 95%, about 96%, about 97%, about 98%, or about 99% of a full-length FGE polypeptide (SEQ ID NO: 2). Preferably, the fragment is biologically active fragment. As used herein, the term “biologically active fragment,” refers to a polypeptide that has an amino-terminal and/or carboxy-terminal deletion(s) and/or internal deletion(s), but where the remaining amino acid sequence is identical to the corresponding positions in the sequence to which it is being compared (e.g. a full-length human FGE of the present invention) and that retains at least some of the activity of the full-length polypeptide. In some embodiments, the biologically active fragment is a biologically active FGE fragment.

In the present invention, a furin cleavage site in eukaryotic, preferably human FGE has been identified, and modified to prevent furin cleavage of eukaryotic FGE. According to the invention, one or more of the codons encoding the furin cleavage site is altered, for example, by site-directed mutagenesis, to prevent recognition of the cleavage site by furin. Preferably, one or more codons are altered to disrupt the cleavage site. Since the minimal furin recognition site is for example in human RYSR (SEQ ID NO: 48), any modification that disrupts the RYSR pattern in human FGE is within the scope of the present invention. This also apply to the extended furin cleavage recognition motif.

The invention also includes degenerate nucleic acids which include alternative codons to those present in the native materials. For example, serine residues are encoded by the codons TCA, AGT, TCC, TCG, TCT and AGC. Thus, it will be apparent to one of ordinary skill in the art that any of the serine-encoding nucleotide triplets may be employed to direct the protein synthesis apparatus, in vitro or in vivo, to incorporate a serine residue into an elongating FGE polypeptide. Similarly, nucleotide sequence triplets which encode other amino acid residues include, but are not limited to: CCA, CCC, CCG and CCT (proline codons); CGA, CGC, CGG, CGT, AGA and AGG (arginine codons); ACA, ACC, ACG and ACT (threonine codons); AAC and AAT (asparagine codons); and ATA, ATC and ATT (isoleucine codons). Other amino acid residues may be encoded similarly by multiple nucleotide sequences. Thus, the invention embraces degenerate nucleic acids that differ from the biologically isolated nucleic acids in codon sequence due to the degeneracy of the genetic code.

The terms “isolated”, “purified”, and “biologically pure” refer to material which is substantially or essentially free from components which normally accompany it as found in its native state, such as for example in an intact biological system. Isolated DNA exhibits a free 3′ end OH group and on its 5′ end a phosphate group which does not occur in nature. As used herein with respect to nucleic acids, the term “isolated” means: (i) amplified in vitro by, for example, polymerase chain reaction (PCR); (ii) recombinantly produced by cloning; (iii) purified, as by cleavage and gel separation; or (iv) synthesized by, for example, chemical synthesis. An isolated nucleic acid is one which is readily manipulated by recombinant DNA techniques well known in the art. Thus, a nucleotide sequence contained in a vector in which 5′ and 3′ restriction sites are known or for which polymerase chain reaction (PCR) primer sequences have been disclosed is considered isolated but a nucleic acid sequence existing in its native state in its natural host is not. An isolated nucleic acid may be substantially purified, but need not be. For example, a nucleic acid that is isolated within a cloning or expression vector is not pure in that it may comprise only a tiny percentage of the material in the cell in which it resides. Such a nucleic acid is isolated, however, as the term is used herein because it is readily manipulated by standard techniques known to those of ordinary skill in the art.

As used herein with respect to polypeptides, the term “isolated” means separated from its native environment in sufficiently pure form so that it can be manipulated or used for any one of the purposes of the invention. Thus, isolated means sufficiently pure to be used (i) to raise and/or isolate antibodies, (ii) as a reagent in an assay, (iii) for sequencing, (iv) as a therapeutic, etc.

The term “conditions permitting the expression” refers to expression of FGE or functional variant or a fragment thereof or polynucleotides introduced in accordance with of the invention (e.g., transfected, infected, or transformed) into an insect cell or for the production of a polynucleotide and/or polypeptide of the invention.

The terms “induce”, “inhibit”, “increase”, “decrease”, “lower”, “affect”, “modulate” or the like, which denote quantitative differences between two states, refer to at least statistically significant differences between the two states and do not necessarily indicate a total elimination of the expression or activity such as cleavage by furin of a FGE variant polypeptide or peptide. Such terms are applied herein to, for example, levels of expression, and levels of activity, which are compared to wt human FGE (SEQ ID NO:1 or 2).

The term “eukaryotic” cell shall refer to a nucleated cell or organism, encompassing but not limited to insect, plant, algae, fungus, mammalian and animal.

### DETAILED DESCRIPTION

Before the invention is described in detail, it is to be understood that this invention is not limited to the particular component parts of the devices described or process steps of the methods described as such devices and methods may vary. It is also to be understood that the terminology used herein is for purposes of describing particular embodiments only, and is not intended to be limiting. It must be noted that, as used in the specification and the appended claims, the singular forms “a,” “an”, and “the” include singular and/or plural referents unless the context clearly dictates otherwise. It is moreover to be understood that, in case parameter ranges are given which are delimited by numeric values, the ranges are deemed to include these limitation values.

The present invention relates to a process for producing high amounts of eukaryotic FGE enzyme in insect cells. Expression of full length eukaryotic FGE which can use glutathione in vitro can be provided by expressing FGE variants having a non-functional furin cleavage motif and subsequently these FGE variants can be used for in vitro generation of aldehyde tags under mild e.g. non-denaturating conditions. In one aspect the present invention relates to a process for producing eukaryotic C-α-formylglycine Generating Enzyme (FGE) or a functional variant or FGE fragment thereof having Cα-formylglycine generating activity or a fragment thereof, comprising: (i) culturing an insect cell containing an isolated polynucleotide encoding the eukaryotic FGE enzyme or a functional variant or a fragment thereof under conditions permitting the expression of FGE or functional variant or a fragment thereof; (ii) obtaining the produced FGE polypeptide or a functional variant or FGE fragment thereof, i.e. the polypeptide of step (ii).

The present invention is based on the surprising finding that the inventors demonstrate for the first time that the truncated form of FGE is generated intracellular by limited proteolysis mediated by proprotein convertase(s) (PCs) along the secretory pathway. The cleavage site is represented by the sequence RYSR⁷²↓ (SEQ ID NO: 48), a motif that is conserved in higher eukaryotic FGEs implying important functionality; see Table 2. Residues R69 and R72 are critical, as their mutation abolishes FGE processing. Furthermore, residues Y70 and S71 confer an unusual property to the cleavage motif such that endogenous as well as overexpressed FGE is only partially processed also the FGE is cleaved by furin, PACE4 and PC5a. Furthermore the surrounding amino acids, i.e. up to one, two three, four, five or six amino acids before the RYSR and one, two, three, four, five, six, seven or eight amino acids after the last R are also important to confer furin function and belong to the non-canonical furin cleavage recognition sequence/motif. Processing is disabled in furin-deficient cells but fully restored upon transient furin expression, indicating that furin is the major protease cleaving FGE. Furin is a calcium dependent serine endoprotease that processes numerous proproteins of different secretory pathways into their mature forms by cleaving at the carboxyl side of the recognition sequence, R-Xaa-(K/R)-R (SEQ ID NO: 49), where Xaa can be any amino acid, the furin cleavage motif of the present invention is listed and discussed below as well as in SEQ ID NO:45 and/or SEQ ID NO: 48.

As mentioned above, the full length FGE exhibits a furin-cleavage motif which is conserved through eukaryotes; see Table 2 which when non-functional allows expression of full length FGE instead of N-terminally truncated FGE.

Thus, in a preferred embodiment, for the production of eukaryotic full length (fl) FGE (34-374 aa), the polynucleotide encoding an eukaryotic fl FGE variant or a fragment thereof comprises a furin cleavage motif in the N-terminal region compared to the human fl FGE wild type (SEQ ID NO:2) which is non-functional, wherein the amino acid numbering of the fl FGE variant or fragment thereof corresponds to human FGE amino acid (SEQ ID NO:2).

Preferably, FGE variants will be sufficiently similar to the amino acid sequence of the preferred polypeptides of the present invention, in particular to FGE full length polypeptide as described in the Examples. Such variants generally retain the functional activity to bind to the cognate ligand of the native FGE of the present invention such as having the ability to use glutathione for their enzymatic activity in vitro. Variants include polypeptides that differ in amino acid sequence from the native and wt hydrophobic polypeptide, respectively, by way of one or more amino acid deletion(s), addition(s), and/or substitution(s). These may be naturally occurring variants as well as artificially designed ones.

Modifications suitable for inactivating the furin cleavage site includes amino acid substitutions, deletions, additions, or combinations of these, that alter the amino acid sequence RYSR to disrupt the furin cleavage site pattern RYSR, particularly disrupting the pattern. For further guidance for introducing amino acid exchange in order to generate suitable mutations see M. J. Betts, R. B. Russell. Amino acid properties and consequences of substitutions. In Bioinformatics for Geneticists, M. R. Barnes, I. C. Gray eds, Wiley, 2003; which is incorporated herein by reference in its entirety.

In one embodiment, the insect cells heterologously expresses the isolated polynucleotide. As used herein, the term “heterologous(ly)” means (a) obtained from a cell or an organism through isolation and introduced into another cell or organism, as, for example, via genetic manipulation or polynucleotide transfer, and/or (b) obtained from a cell or an organism through means other than those that exist in nature, and introduced into another cell or organism, as for example, through cell fusion, induced mating, or transgenic manipulation. A heterologous material may, for example, be obtained from the same species or type, or a different species or type than that of the organism or cell into which it is introduced. Preferred in accordance with the present invention is the heterologous expression of eukaryotic, preferably human wt full length, truncated i.e. delta72 FGE, variants or fragments thereof.

The insect cells used in the method of the present invention are preferably selected from the group consisting of insect cells derived from Spodoptera frugiperda, Trichoplusia ni, Plutella sylostella, Mand uca sextra and Mamestra brassicae; preferably the insect cell is selected from the group consisting of SF9, SF21, High Five™ Cells (BTI-TN-5B1-4) KCl, Drosophila SFM and Mimic™ Sf9 insect cells.

Other parental cell lines available for production of stably transfected cell lines include D.Mel-2 cells, KCl, IPLB-Sf21, BTI-Tn5B1-4, BTI-MG-1, Tn368, Ld652Y, and BTI-EAA, any cell lines derived from the cell lines listed here, as well as any cell line susceptible to baculovirus infection. Those skilled in the art would appreciate that, in order to meet their unique expression needs, this method is applicable to cell lines not specifically listed.

Insect cells transiently or stably expressing a polypeptide of interest can be generated by various means such as baculovirus infection, transfection via electroporation or with lipid-based agents and are well known to the skilled person and are commercially available by Novagen (Merck) and Invitrogen. Also vectors, cosmids, BACs, different media compositions for transfection and maintenance as well as protocols and troubleshooting as well as further reading is described in detail in the manuals available from Invitrogen and Novagen (Merck) in addition in WO0166696 an apoptotic resistant Sf9 insect cell line for expressing high amounts of proteins is described, see also U.S. Pat. No. 5,728,580 as well as Dyring C. et al., Journal 10 (2011) 28-35; McCarroll, L. and L. A. King. Current Opinions in Biotechnology 8 (1997) 590-594. In accordance with the present invention, high amounts of FGE variants are produced by the inventive process, i.e. more than 30, preferably 40, more preferably >50 mg FGE/liter culture medium.

The invention also provides a method for small as well as large-scale recombinant FGE full length or functional variants or FGE fragments thereof peptide and/or polypeptide production using the baculovirus expression system allowing increased yields of the wanted peptide and/or polypeptide. The invention provides a method to produce a recombinant FGE full length or functional variant or FGE fragment thereof in insect-cell culture which comprises selecting a recombinant baculovirus encoding said protein, growing insect cells in growth medium in a culture vessel, and infecting the cells with a multiplicity of infection of at least 0.001. Thus, in one embodiment the process further comprises the following steps which are to be conducted prior to step (i) of claim 1: (ia) infecting the cell with a recombinant baculovirus, wherein the virus containing an isolated polynucleotide encoding the eukaryotic FGE or a functional variant thereof or a fragment thereof; (ib) producing an infected insect cell capable of expressing FGE or a variant thereof.

A preferred embodiment of the invention provides a method to produce a recombinant protein in insect-cell cultures which comprises selecting a recombinant baculovirus encoding said protein, growing the insect cells in growth medium in a culture vessel with a sufficient volume to contain at least 10 ml, 250 ml to 2 liters and infecting the insect cells with an inoculum of at least one baculovirus with an m.o.i of at least 0.01 PFU of said baculovirus/cell. In a preferred embodiment the baculovirus is Autographa californica multicapsid nucleo polyhedrovirus (AcMNPV) or Bombyx mori nuclear polyhedrovirus (BmNPV). Any suitable baculovirus can be used in accordance with the present invention, as long the virus can infect insect cells and lead to the expression of an heterologous recombinant FGE variant in a sufficient amount.

The invention provides a method wherein multiplicities of infection are used that are considerably lower than for example the m.o.i. of 1-5 leading to an asynchronously infected culture. A preferred embodiment of the method according to the invention comprises growing the cells in a culture vessel with a sufficient volume to contain at least 10, more preferably at least 20, more preferably at least 50 or 250 liters growth medium, thereby allowing scaling-up of baculovirus cultures expressing heterologous proteins. One can for example use a culture vessel with a volume that is larger than needed for the volume of growth medium that is present e.g. one can use 100 L culture vessels to cultivate 20-70 liters cell-culture. A preferred embodiment of the method according to the invention comprises infecting the cells at a cell density of 1×10<5> to 5×10<6> cells/ml, more preferably at 5×10<5> to 1.5×10<6> cells/ml, thereby keeping the actual volume of the virus inoculum within easily manageable limits. Yet another embodiment of the method according to the invention comprises infecting the cells with an m.o.i. such as 0.00025, 0.0005, 0.001, 0.002, 0.003, 0.004, 0.005, 0.006, 0.007, 0.008, 0.009, 0.01, 0.02, 0.03, 0.04, 0.05, 0.06, 0.07, 0.08, 0.09, 0.1, 0.2, 0.3, 0.4, 0.5, 0.6, 0.7, 0.8, 0.9, 1, 2, 3, 4, 5, 6, 7, 8, 9, 10, 11, 12, 13, 14, 15, 16, 17, 18, 19, 20, 30, 40, 50, 60, 70 80, 90 or 100, whereby preferably the inoculum is kept as small as possible.

There are a number of commercial systems available for expressing recombinant proteins using baculovirus, including flashBAC™ (Oxford Expression Technologies EP 1 144 666), BackPack™ (BD Biosciences Clontech), BacVector® 1000/2000/3000 (Novagen®), BAC-TO-BAC® (Invitrogen™ U.S. Pat. No. 5,348,886), and BaculoDirect™ (Invitrogen™). All of these systems are based on the principle of expressing recombinant proteins by placing them under the control of the very late baculovirus promoters polh or p10. Furthermore, systems allowing for inducible control of protein expression, particularly, for repression of recombinant protein expression during virus amplification as part of the scale up phase, or for repression of baculovirus production during the protein production phase are described in WO2013014256, the disclosure content of which is hereby incorporated by referencing herein. These and further systems are suitable for production of high yields of recombinant protein even in large industrial-scale cell cultures. Further suitable baculovirus-based technology systems are described in Nature Biotechnol. 23: 567-575, wherein the “BacMam” technology uses baculoviruses as vehicles to deliver and inducibly express recombinant proteins in mammalian cells. Corresponding approaches that use baculovirus technology to deliver an expression system into mammalian cells, for inducible expression of a recombinant protein in mammalian cells, have also been disclosed by McCormick et al (J Gen Virol, 2002; 83: 383-394 and J Gen Virol, 2004; 85: 429-439). Further embodiments and examples for baculovirus-cell expression systems and method for generating them are found within the disclosure, definitions, claims and examples of international application PCT/EP2011/050996, such embodiments and examples being henceforth readily incorporated into the present invention by the person of ordinary skill following the disclosure herein. The disclosure of said application, is hereby incorporated by referencing herein.

In accordance with the present invention, the insect cell can express FGE from every known species, in particular eukaryotic, see for example Table 2 wherein it is evident, that all eukaryotic listed species exhibit an N-terminal sequence contrary to bacteria-derived FGE. However, in a preferred embodiment of the present invention, the eukaryotic FGE species is selected from the group consisting of mammalian, human, fungus, algae and insect. In a more preferred embodiment, the species is human.

In view to the above, the skilled person will appreciate that every FGE produced by the insect cells can be distinguished by its post-translational modifications, such as N-Acetylglucosamine, phosphorylation, where sialic acid, hexuronic acid, and sulfate and/or glycosylation pattern, which differs from human post-translational modifications; see inter alia for further reading Lim et al., 2011, Biotechnol Prog., 5, 1390-6. doi: 10.1002/btpr.662 as well as Kenny et al., Methods Mol Biol. 2013; 988:145-67. doi: 10.1007/978-1-62703-327-5 9. These FGE polypeptides will in most cases exhibit insect specific post-translational modifications which are not encoded by sequence. Thus, in another aspect the invention naturally extends to eukaryotic C-α-formylglycine Generating Enzyme (FGE) or a functional variant thereof having Cα-formylglycine generating activity or a FGE fragment obtainable by the process of invention, wherein the obtained eukaryotic FGE polypeptide exhibit insect-specific post-translational modifications or is at least distinguishable by the post-translational modifications from human-expressed FGE polypeptides and/or fragments thereof. In a preferred embodiment, human FGE polypeptides are expressed.

As outlined above, the process of the present invention makes use of the discovery made by the inventors that the furin cleavage motif having at least a core motif of the amino acid formula: R-Y-S-R corresponding to human FGE amino acid (SEQ ID NO: 2) aa 69-72. However, as also shown in Example 11 amino acids around the core motif are also important for proteolytic processing of FGE. This finding can be further underlined by the fact that cleavage efficiency of mammalian PCs has been shown to be directly dependent on ˜20 amino acid residues surrounding the cleavage site and especially the positions P4 to P1′ (RYSR72↓E in FGE) (SEQ ID NO: 48) are important, see for further reading Turpeinen H. et al., BMC Genomics 2011, 12:618, the disclosure content of the reference is incorporated herein by its entirety. Thus, according to the present invention, it is prudent to expect that any amino acid mutation in these reaction leads to increased or decreased or inhibited cleavage by furin on the FGE full length enzyme.

Thus, in one aspect the present invention relates to an eukaryotic FGE polypeptide variant having Cα-formylglycine generating activity, wherein said variant comprises an amino acid sequence comprising the furin cleavage motif of the invention and having one or more amino acid modification such as an exchange in the furin-cleavage motif, wherein the modification results in i) a FGE variant having a non-functional or non-cleavable furin cleavage motif; or ii) a FGE variant having an optimized furin cleavage motif, wherein the one or more of the amino acid modification is located in the furin core motif as defined above, or the amino acid modification takes place in the extended furin-cleavage motif comprising: Xₙ₋₆-RYSR-Xₙ₊₈, corresponding to human FGE amino acid (SEQ ID NO:2) aa 63-80, wherein (iii) Xₙ₋₆ can be SSAAAH in position 63 to 68, (iv) Xₙ₊₈ is at least EANAPGPV, in position 73 to 80 (SEQ ID NO: 45).

The term “modification” in connection with FGE polypeptides or peptides of the present invention is defined as deletion, substitution or introduction of at least one, two, three, or four amino acid, preferably at least one amino acids and/or any other modifications of the amino acid which will result in a non-cleavable furin motif or in an improved cleavable motif Peptides and proteins can be derivatized either naturally or synthetically; such modifications can include, but are not limited to, glycosylation, phosphorylation, acetylation, myristylation, prenylation, palmitoylation, amidation and/or addition of glycerophosphatidyl inositol; Suitable modifications of amino acids are well known for the skilled person; see e.g. Basle et al., Chem Biol. 2010 Mar. 26; 17(3):213-27, methods for generating modifications onto amino acids are described in WO2000078791; the disclosure content of these publications are incorporated herein by reference in its entirety. Preferably the modification is an amino acid exchange.

As shown in the Examples, amino acid substitution of glutamic acid (E) to proline (P) in the furin cleavage motif (Xₙ₊₈) results in a non-functional FGE variant. In accordance with the above, the variant FGE polypeptide exhibit an amino acid modification in (i) Xₙ₋₆ i.e. in SSAAAH, wherein any of the amino acid can be changed to an uncharged small and/or hydrophobic, positive and/or polar charged amino acid, and/or a modification as defined herein and/or (ii) Xₙ₊₈ is a non-polar and/or polar, acid and/or basic amino acid and/or a modification as defined herein; wherein at least one amino acid in residue in (i) to (ii) is changed compared to the wild type.

Furthermore, in one embodiment, the Xₙ₋₆-RYSR-Xₙ₊₈ motif has been modified. In accordance with the present invention, “modifying a motif” is used interchangeably with the term “modification” as defined above. Further suitable modifications of amino acids are well known for the skilled person; see e.g. Basle et al., Chem Biol. 2010 Mar. 26; 17(3):213-27, methods for generating modifications onto amino acids are described in WO2000078791; the disclosure content of these publications are incorporated herein by reference in its entirety. Preferably, amino acid substitution with another amino acid is used.

In a preferred embodiment, the modified FGE polypeptides of the invention include amino acid modifications in the FGE amino acid sequence, wherein one or more, and preferably two or more, preferably three or more of the amino acid residues 69-RYSR-72 and/or in the amino acid residues 63-SSAAAH-RYSR-EANAPGPV-80 (SEQ ID NO: 45) are substituted with a different amino acid residue and/or otherwise modified, disrupting the furin cleavage motif pattern. Preferably, one or more arginine is substituted with a non-basic, more preferably a neutral amino acid. By way of example, substitution of one arginine (R) results in the disrupted sequence XYSR or RYSX; substitution of two arginines results in the disrupted sequence XYSX or YS, wherein X can be any amino acid which is not positively charged such as substituting R to alanine (A), proline (P), glycine (G), valine (V), isoleucine (I) or leucine (L) or even negatively charged as glutamic acid (E) or aspartic acid (D) resulting in decreased, inhibited, lowered binding between the FGE cleavage motif and furin. In one embodiment, the sequence RYSR motif has been deleted in the modified FGE polypeptides of the present invention.

In one embodiment, the modified FGE polypeptides of the invention include deletions of one or more, preferably two or more of the amino acid residues 69-RYSR-72 of SEQ ID NO: 48 to disrupt the RXXR furin cleavage pattern. For example, deletion of tyrosine (Y) or serine (S) results in the disrupted sequence RSR or RYR. In one embodiment, at least two amino acids within the furin cleavage site are altered to remove amino acids tyrosine (Y) and serine (S) that can be recognized by furin.

Depending on the use, i.e. generation of a FGE variant having increased binding affinity to furin, an amino acid substitution can be chosen which will increase, support or optimize the binding between the FGE cleavage motif and furin, such as substituting Y to tyrosine (K), serine (S), phenylalanine (F) or histidine (H), S to alanine (A), glycine (G), valine (V), isoleucine (I) or leucine (L) arginine (R) or lysine (K). Preferably, tyrosine (Y) is changed to an uncharged or positively charged amino acid or residue and/or serine (S) to an uncharged or positively charged amino acid or residue.

Depending on the use, i.e. generation a FGE variant having decreased binding affinity to furin, an amino acid substitution can be chosen which will decrease, i.e. reduce, lower, retard, inhibit the binding between the FGE cleavage motif and furin, such as substituting Y to aspartic acid (D), or glutamic acid (E), alanine (A), glycine (G), proline (P), valine (V), isoleucine (I) or leucine (L).

Preferably the polypeptide exhibit an amino acid exchange of R to a polar or non-polar amino acid and/or small amino acid or Y is exchanged/substituted to a basic, small or hydrophobic amino acid, S is exchanged to a negative, hydrophobic or small amino acid wherein the amino acid exchange/substitution results in a FGE variant comprising a non-functional furin cleavage motif or in an improved furin cleavage motif.

In a preferred embodiment the amino acid substitution leads to a substantial changes in function or immunological identity which is made by selecting substitutions that are less conservative than those mentioned in the definition section, i.e. selecting residues that differ more significantly in their effect on maintaining (a) the structure of the polypeptide backbone in the area of the substitution, for example as a sheet or helical conformation, (b) the charge or hydrophobicity of the molecule at the target site, or (c) the bulk of the side chain. The substitutions which in general are expected to produce the greatest changes in the protein properties are those in which: (a) the hydrophilic residue, e.g., seryl or threonyl, is substituted for (or by) a hydrophobic residue, e.g., leucyl, isoleucyl, phenylalanyl, valyl or alanyl; Tryptophan, Tyrosinyl (b) a cysteine or proline is substituted for (or by) any other residue; (c) a residue having an electropositive side chain, e.g., lysyl, arginyl, or hystidyl, is substituted for (or by) an electronegative residue, e.g., glutamyl or aspartyl; or (d) a residue having a bulky side chain, e.g., phenylalanine, is substituted for (or by) one not having a side chain, e.g., glycine, in this case, or (e) by increasing the number of sites for post-translational modifications which will lead to the desired effect. In accordance with the present invention the post-translational covalent bond modifying process is selected from the group consisting of a phosphorylation, glycosylation, carboxylation, ADP-ribosylation, methylation, isoprenylation, acylation and/or sulfation.

The modified FGE polypeptides of the invention also include amino acid additions to the FGE amino acid sequence where one or more amino acid residues are inserted into the furin cleavage sequence 69-RYSR-72 (SEQ ID NO: 48), disrupting the RXXR pattern. For example, one or two or more amino acids can be inserted, such as in the sequence 69-RYZnR-72 where Z is not S, and n is 1 or more or 69-RZnSR-72 where Z is not Y, and n is 1 or more; and preferably two or more amino acids can be inserted. Preferably, n is 2, 3, 4, or 5, and Z is a neutral amino acid.

The skilled person is well aware of suitable techniques for generating amino acid substitutions, such as site directed mutations as shown in the Examples. The amino acid substitution provides for an amino acid residue that fails to provide the requisite combination of charge and size and/or pKₐ of an Arginine side chain for catalysis of a phosphotransfer reaction.

It is understood that one way to define the disclosed FGE variants or fragments thereof herein is to define them in terms of homology/identity to specific known sequences. Specifically disclosed are variants of human full length FGE having a variation in one or 2, 3, 4, 5, 6, 7, 8, 9, 10, 11, 12, 13 and/or 14 amino acid position(s) corresponding to human amino acid sequence position 63 to 80, and fragments/peptides thereof comprising at least the modified sequence. In addition, these FGE variants herein disclosed have at least, 60%, 65%, 70% or at least 75% or at least 80% or at least 85% or at least 90% or at least 95% homology to the human FGE of SE ID NO:1 specifically recited herein. Those of skill in the art readily understand how to determine the homology of two proteins.

Based on the teaching given herein the skilled person is well in the position to provide further suitable FGE variants. Apart from amino acid substitutions the amino acids located in the furin cleavage motif i.e. four up to 18 amino acid residues, can also be modified by posttranslational modifications which leads to the desired effect of providing a FGE enzyme or fragment thereof wherein the cleavage motif is modified and therefore is improved for catalytic cleavage or is no longer a substrate for the furin or furin-like proteases.

Thus, in one embodiment the FGE full length polypeptide and variants or fragments thereof according to the invention can be defined by its distinct properties/characteristics such as a particular size in kDa, amino acid sequence, enzymatic activity which can be determined with techniques well known in the art such as SDS-PAGE, IEF, UV, CD, fluorescence spectroscopy, MALDI ToF mass spectrometry, Sequencing, ELISA and NMR, see also the Examples. Further suitable methods such as diagnostically and therapeutically, as well as an immunohistochemical assays, such as Western blots, ELISA, radioimmunoassays, immunoprecipititations, cell fluorescence activated cytometry and/or cell sorting (FACS) magnetic activated cell sorting (MACS) or other immunochemical assays known in the art.

General information and protocols are disclosed in Raem, Arnold M. Immunoassays. 1st ed., Munich; Heidelberg: Elsevier, Spektrum Akademischer Verlag., 2007; David Wild (Ed.): The Immunoassay Handbook. 3rd ed. Elsevier Science Publishing Company, Amsterdam, Boston, Oxford 2005.

In one embodiment of the present invention, the polypeptide of the invention in particular the full length FGE variants having one or more amino acid exchanges in the furin-cleavage motif exhibit at least one of the following characteristics:
(a) is at least a 41 kDa+/−3 kDa protein (SDS-PAGE) and/or has approximately a 55aa N-terminal extension compared to prokaryotic FGE;
(b) exhibit in vitro formlyglycine formation activity;
(c) is stable during chromatographic purification process;
(d) exhibits the N-terminal sequence EAN (Glu-Ala-Asn);
(e) exhibits an amino acid sequence having 85% or more identity human FGE amino acid sequence (SEQ ID NO: 2); and/or;
(f) catalyze thiol-to-aldehyde oxidation of cysteine residues in the presence of glutathione.

As evident from Example 19, the inventive process not only leads to the production of full length FGE in monomeric form, but also in dimeric form. These fl FGE dimers are more stable than monomeric fl FGE forms and are still capable of using GSH as a reducing agent.

In this context, it should be understood that 41 kDa as well as 37 kDa band visible in SDS PAGE are not absolute numerical values. The inventors observed that also some smaller bands of the two most prominent bands are present, maybe due to the cleavage by peptidases. Aminopeptidases catalyze the cleavage of amino acids from the amino terminus of protein or peptide substrates. Thus, 1, 2, 3, 4, 5, 6, 7, 8, 9, 10, 11, 12, 13, 14, 15, 16, 17, 18, 19, or 20 amino acid may be cleaved off on the N-terminus of FGE, either the delta 72 or the full length variant. Alternatively or additionally, also carboxypeptidases which are protease enzymes that hydrolyze (cleaves) a peptide bond at the carboxy-terminal (C-terminal) end of a protein or peptide. Humans, animals, and plants contain several types of carboxypeptidases that have diverse functions ranging from catabolism to protein maturation. Thus, 1, 2, 3, 4, 5, 6, 7, 8, 9, 10, 11, 12, 13, 14, 15, 16, 17, 18, 19, or 20 amino acid may be hydrolyzed on the C-terminus of FGE, either the delta 72 or the full length variant.

In addition, a shift of up to 3 kDa of 37 kDa and/or 41 kDa band in a SDS PAGE gel corresponding to full length or delta 72 FGE protein may also be due to the occurrence of different post-translational modifications which may or may not be present on FGE polypeptides, variants or fragments thereof. Individual FGE polypeptides may differ in respect to the extent, to the complexity, to the nature, to the antennarity and to the order of attached glycosyl-, sialyl-, and acetyl groups. Even charged anorganic groups like phosphate and sulphate may contribute to the nature of a specific FGE polypeptide. However, even full length FGE or delta 72 FGE does not exhibit exactly 37 kDa or 41 kDa, respectively, they could still be defined by their amino acid sequence while having a different, i.e. distinct isoelectric point. Isoelectric points as revealed for example by Isoelectric Focussing (IEF) gels or distinct number of charges as revealed for example by Capillary Zone Electrophoresis (CZE).

In a preferred embodiment the variant i.e. modified FGE polypeptide comprises at least one of the substitutions selected from the group consisting of SEQ ID NO:8 (R69A); SEQ ID NO: 10 (R69K), SEQ ID NO:12 (Y70A), SEQ ID NO: 14 (Y70K), SEQ ID NO: 16 (Y70F), SEQ ID NO: 18 (Y705), SEQ ID NO:20 (S71A); SEQ ID NO:22 (S71R), SEQ ID NO:24 (R72K); SEQ ID NO:26 (R72A), SEQ ID NO:4, (R69A/R72A), SEQ ID NO:29 (Y70A/S71R) and SEQ ID NO: 31 (E73P), or a combination of the mentioned amino acid substitutions thereof. In addition, the amino acid sequence of the variant comprises of an amino acid sequence having at least a degree of identity to SEQ ID NO:2 of at least 70%, such as at least 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% and/or 100%.

As this specification discusses various polypeptides and polypeptide sequences it is understood that the nucleic acids that can encode those polypeptide sequences are also disclosed. This would include all degenerate sequences related to a specific polypeptide sequence, i.e. all nucleic acids having a sequence that encodes one particular polypeptide sequence as well as all nucleic acids, including degenerate nucleic acids, encoding the disclosed variants and derivatives of the protein sequences. Thus, while each particular nucleic acid sequence may not be written out herein, it is understood that each and every sequence is in fact disclosed and described herein through the disclosed polypeptide sequences.

Useful fragments of the polynucleotides of the invention include probes and primers. In one aspect, the present invention relates to a primer for the generation of the FGE variants as listed in the Table 1 below, comprising or consisting of one of the following sequences

TABLE 1
FGE Primer sequences for generating
FGE fl variants

| SEQ | | Mutation |
|---|---|---|
| ID | | amino acid |
| NO: | Nucleotide sequence | exchange |
| 7 | TCGGCAGCCGCTCACGCATACTCGCGGGAGGCT | R69A |
| 9 | TCGGCAGCCGCTCACAAATACTCGCGGGAGGCT | R69K |
| 11 | GCAGCCGCTCACCGAGCCTCGCGGGAGGCTAAC | Y70A |
| 13 | GCAGCCGCTCACCGAAAGTCGCGGGAGGCTAAC | Y70K |
| 15 | GCAGCCGCTCACCGATTCTCGCGGGAGGCTAAC | Y70F |
| 17 | GCAGCCGCTCACCGATCCTCGCGGGAGGCTAAC | Y70S |
| 19 | GCCGCTCACCGATACGCGCGGGAGGCTAACGCT | S71A |
| 21 | GCCGCTCACCGATACAGGCGGGAGGCTAACGCT | S71R |
| 23 | GCTCACCGATACTCGAAGGAGGCTAACGCTCCG | R72K |
| 25 | GCTCACCGATACTCGGCGGAGGCTAACGCTCCG | R72A |
| 27 | GCTCACGCATACTCGGCGGAGGCTAACGCTCCG | R69A/R72A |
| 28 | GCCGCTCACCGAGCCAGGCGGGAGGCTAACGCT | Y70AS71R |
| 30 | CACCGATACTCGCGGCCGGCTAACGCTCCGGGC | E73P |
| 14 | CCGGAATTCAGCCAGGAGGCCGGGACC | Bac-wt no |
| | | ER signal |

These primers on itself, or modified to the specific need can be used, for example, in PCR methods to generate the FGE polypeptide variants and/or to amplify and detect the presence of modified FGE polynucleotide(s) in vitro, as well as in Southern and Northern blots for analysis of polynucleotides encoding protease resistant or protease sensitive FGE. Cells transiently or stably overexpressing the protease resistant or protease sensitive FGE polynucleotide molecules of the invention can also be identified by the use of such probes or those described in the Examples. Methods for the production and use of such primers and probes are known.

Other useful fragments include antisense or sense oligonucleotides comprising a single-stranded nucleic acid sequence capable of binding to a target FGE polypeptide variants mRNA (using a sense strand) or DNA (using an antisense strand) sequence.

In one aspect the present invention relates to an isolated nucleic acid derived from eukaryotic organism, comprising a nucleic acid sequence that code for the polypeptide as mentioned above. The polynucleotide of the invention encoding the above described FGE variant may be, e.g., DNA, cDNA, RNA or synthetically produced DNA or RNA or a recombinantly produced chimeric nucleic acid molecule comprising any of those polynucleotides either alone or in combination. Preferably said polynucleotide is part of a vector. Thus, in another aspect the present invention relates to a vector comprising the polynucleotide or the primer or a functional portion thereof. Such vectors may comprise further genes such as marker genes which allow for the selection of said vector in a suitable host cell and under suitable conditions. Preferably, the polynucleotide of the invention is operatively linked to expression control sequences allowing expression in prokaryotic or eukaryotic cells. Expression of said polynucleotide comprises transcription of the polynucleotide into a translatable mRNA. Regulatory elements ensuring expression in eukaryotic cells, preferably mammalian or insect cells, are well known to those skilled in the art. They usually comprise regulatory sequences ensuring initiation of transcription and optionally poly-A signals ensuring termination of transcription and stabilization of the transcript. Additional regulatory elements may include transcriptional as well as translational enhancers, and/or naturally associated or heterologous promoter regions.

In this respect, the person skilled in the art will readily appreciate that the polynucleotides encoding at least the full length FGE polypeptide having at least one mutation in the furin motif, i.e. the variant or may encode a fragment of the FGE variant. Likewise, said polynucleotides may be under the control of the same promoter or may be separately controlled for expression. Possible regulatory elements permitting expression in prokaryotic host cells comprise, e.g., the PL, lac, tip or tac promoter in E. coli, and examples for regulatory elements permitting expression in eukaryotic host cells are the AOX1 or GAL1 promoter in yeast or the CMV-, SV40-, RSV-promoter (Rous sarcoma virus), CMV-enhancer, SV40-enhancer or a globin intron in mammalian and other animal cells.

Beside elements which are responsible for the initiation of transcription such regulatory elements may also comprise transcription termination signals, such as the SV40-poly-A site or the tk-poly-A site, downstream of the polynucleotide. Furthermore, depending on the expression system used leader sequences capable of directing the polypeptide to a cellular compartment or secreting it into the medium may be added to the coding sequence of the polynucleotide of the invention and are well known in the art. The leader sequence(s) is (are) assembled in appropriate phase with translation, initiation and termination sequences, and preferably, a leader sequence capable of directing secretion of translated protein, or a portion thereof, into the periplasmic space or extracellular medium. Optionally, the heterologous sequence can encode a fusion protein including a C- or N-terminal identification peptide imparting desired characteristics, e.g., stabilization or simplified purification of expressed recombinant product. In this context, suitable expression vectors are known in the art such as Okayama-Berg cDNA expression vector pcDV1 (Pharmacia), pCDM8, pRc/CMV, pcDNA1, pcDNA3 (Invitrogen), or pSPORT1 (GIBCO BRL).

Preferably, the expression control sequences will be eukaryotic promoter systems in vectors capable of transforming or transfecting eukaryotic host cells, but control sequences for prokaryotic hosts may also be used. Once the vector has been incorporated into the appropriate host, the host is maintained under conditions suitable for high level expression of the nucleotide sequences, and, as desired, the collection and purification of the FGE variants or fragments thereof.

Suitable insect expression system, i.e. vectors, promoters and the like are described in detail above.

The modified FGE polypeptides to be expressed in host cells can also be a fusion protein, which includes the FGE polypeptide and at least one heterologous polypeptide. As discussed below, heterologous polypeptides can be fused to the FGE polypeptide to facilitate, for example, secretion, stability, purification, and/or targeting of the modified FGE polypeptide. Examples of fusion proteins provided by the present invention includes fusions of modified FGE polypeptides with, for example Fc polypeptides and leucine zipper domains to promote the oligomerization of the FGE polypeptides as described in WO 00/29581.

As described above, the polynucleotide of the invention can be used alone or as part of a vector to express the (poly)peptide of the invention in cells, for, e.g., protein production, research tool, gene therapy or diagnostics of diseases related to FGE deficient diseases. The polynucleotides or vectors of the invention are introduced into the cells which in turn produce the FGE variant. Gene therapy, which is based on introducing therapeutic genes into cells by ex-vivo or in-vivo techniques is one of the most important applications of gene transfer. Suitable vectors and methods for in-vitro or in-vivo gene therapy are described in the literature and are known to the person skilled in the art; see, e.g., Giordano, Nature Medicine 2 (1996), 534-539; Schaper, Circ. Res. 79 (1996), 911-919; Anderson, Science 256 (1992), 808-813; Isner, Lancet 348 (1996), 370-374; Muhlhauser, Circ. Res. 77 (1995), 1077-1086; Wang, Nature Medicine 2 (1996), 714-716; WO94/29469; WO 97/00957 or Schaper, Current Opinion in Biotechnology 7 (1996), 635-640, and references cited therein. The polynucleotides and vectors of the invention may be designed for direct introduction or for introduction via liposomes, or viral vectors (e.g. adenoviral, retroviral) into the cell. Preferably, said cell is a germ line cell, embryonic cell, or egg cell or derived therefrom, most preferably said cell is a stem cell.

Furthermore, the present invention relates to vectors, particularly plasmids, cosmids, viruses and bacteriophages used conventionally in genetic engineering that comprise a polynucleotide encoding a FGE variant as described; optionally in combination with a polynucleotide of the invention that encodes a purification tag or linker. Preferably, said vector is an expression vector and/or a gene transfer or targeting vector. Expression vectors derived from viruses such as retroviruses, vaccinia virus, adeno-associated virus, herpes viruses, or bovine papilloma virus, may be used for delivery of the polynucleotides or vector of the invention into targeted cell population. Methods which are well known to those skilled in the art can be used to construct recombinant viral vectors; see, for example, the techniques described in Sambrook, Molecular Cloning A Laboratory Manual, Cold Spring Harbor Laboratory (1989) N.Y. and Ausubel, Current Protocols in Molecular Biology, Green Publishing Associates and Wiley Interscience, N.Y. (1994). Alternatively, the polynucleotides and vectors of the invention can be reconstituted into liposomes for delivery to target cells. The vectors containing the polynucleotides of the invention can be transferred into the host cell by well-known methods, which vary depending on the type of cellular host. For example, calcium chloride transfection is commonly utilized for prokaryotic cells, whereas calcium phosphate treatment or electroporation may be used for other cellular hosts; see Sambrook, supra.

The term “host cell”, as used herein, includes any cell type that is susceptible to transformation, transfection, transduction, and the like with a nucleic acid construct or expression vector comprising a polynucleotide of the present invention. “Host cell” is a cell, including but not limited to a eukaryotic or prokaryotic cell, such as mammalian cell, animal cell, insect cell, plant cell, algae cell, fungus cell, bacterial cell or cell of a microorganism into which an isolated and/or heterologous polynucleotide sequence has been introduced (e.g., transformed, infected or transfected) or is capable of taking up exogenous nucleic acid (e.g., by transformation, infection or transfection). In a preferred embodiment, the host cell is selected from the group consisting of mammalian, human, algae, fungus and insect cell. Any cell which is capable of expressing an integrated (in the genome) or a free replicating (e.g. plasmid) transgene or comprises endogenously or heterologously a FGE and/or FGE variant as defined above or a homolog and/or ortholog thereof, having substantially the same functional proteolytic cleavage motif as the mammalian furin-cleavage motif or can be expressed as a FGE enzyme having an N-terminal region which comprises the two conserved cysteine residues which helps to use glutathione for oxidation reaction of cysteine residues in at least in vivo reactions, is suitable in accordance with the meaning of the present invention; see e.g., US patent application 2008/0305523 for suitable fungus cells; U.S. Ser. No. 08/477,559 for suitable plant cells; EP2560479 for suitable algae cells; US 2011/0117643 for suitable animal cells; US patent 20130034897 for suitable bacterial cells as well as EP1050582 for suitable microorganism cells. However, a preferred embodiment in accordance with the present invention is the use of the vector, the host cell or a suitable primer as defined above, in the process of the invention or any method.

A host cell as defined above contains a nucleic acid such as an active gene coding for the respective polypeptide and this nucleic acid is transcribed and translated during culture of the cell in the medium. The gene can be introduced into this host cell as an exogenous gene, preferably with regulation (regulatory) elements (see, e.g., EP-B 0 148 605) or can already be present in the host cell as an active endogenous gene or can become activated as an endogenous non-functional gene. Such an activation of endogenous genes can be achieved by the specific introduction of regulation (regulatory) elements into the genome by homologous recombination, see for further reading international applications WO 91/09955 and WO 93/09222.

As shown in Examples 9 to 11 mammalian cells are suitable to express FGE variants having a non-functional furin cleavage motif at higher amounts than human FGE wild type (aa 34 to 374) which is cleaved within the cell or later upon chromatographic purification process. In order to increase the amount of expressed FGE variant protein or FGE full length wild type protein a host cell which is deficient of furin or furin-like proteolytic activity is used for the process of the present invention. According to the present invention, a furin and/or furin-like proteolytic deficient cell means that the cell, either (a) express a non-functional furin and/or furin-like polypeptide; (b) lacks the gene(s) coding for one or more furin and/or furin-like enzyme(s); (c) express siRNA, lhRNA, shRNA specific to target the mRNA of furin and/or furin-like protease, (d) is maintained in the presence of a furin inhibitor, selected from the group essentially consisting of a complement-binding peptide specific for the cleavage motif defined above or listed in Table 1 or outlined below, RVKR-Chloromethylketone (CMK) or an derivative thereof and/or wherein the furin-like protein is selected from the group consisting of following proteases furin, PC2, PC1/PC3, PC4, PC5/PC6, LPC/PC7/PC8/SPC7PACE4, PCSK9.

Furin-like protease can be any protease which exhibit in vivo, ex vivo or in vitro the ability to bind to the furin cleavage motif of the present invention and facilitates the dissociation of the FGE polypeptide chain leading to a truncated polypeptide chain. For further reading regarding the furin-like protease or furin in particular its mechanism of action, inhibitors, sequences see e.g. Nakayama K et al., Biochem J. 1997 Nov. 1; 327 (Pt 3):625-35. Non-peptidic furin inhibitors containing amidinohydrazone moieties are described in Kibirev V K et al., Ukr Biokhim Zh. 2013 January-February;85(1):22-32 amidinohydrazone-derived inhibitors in Sielaff FBioorg Med Chem Lett. 2011 Jan. 15; 21(2):836-40 and furin and furin-like protease inhibitors in Basak A. et al., J Mol Med (Berl). 2005 November; 83(11):844-55. Furin-derived human peptides known to inhibit furin actions are peptide having the residues 55-62, 50-62, 39-62, 50-83, 55-83, 64-83 and 74-83 in the pro-mouse PC1/3 sequence and residues 54-62, 48-62 and 39-62 of the pro-human furin sequence; see e g. Basak A et al., Biochem J. 2003 July 1; 373(Pt 1):231-9. Generation of knock out of the furin gene either inducible such as tet on/off, via lentiviral or other suitable methods such as transient or inducible expression shRNA, microRNA or RNAi systems are WO2013/006142 or US20090203055 well known to the skilled person and are described in several textbooks and can be commercially obtained from various distributors such as Thermo Fischer, Clontech or Invitrogen.

According to another aspect of the invention, immunogenic fragments of the FGE polypeptide variant or a fragment thereof as described above are provided. The immunogenic fragments may or may not have C_{α}-formylglycine generating activity. Thus, immunogenic fragments which are isolated binding polypeptides are provided which selectively bind a polypeptide encoded by the foregoing nucleic acid molecules of the invention. Preferably the isolated binding polypeptides selectively bind a polypeptide which comprises at least the sequence of amino acids 63 to 83 of SEQ ID NO: 45 i.e. at least SEQ ID No:48, fragments thereof, or any polypeptide variants having a different furin-cleavage motif as described which leads to a different function such as a non-functional cleavage or an improved cleavage described elsewhere herein. Preferred, in accordance with the present invention, an antibody which recognize an epitope of the N-terminal region of human FGE (aa 33 to 72) is provided.

In preferred embodiments, the isolated binding polypeptides include antibodies and fragments of antibodies. Fragments spanning a modified furin cleavage site, including a fragment where the furin cleavage site has been deleted, can be used to generate specific antibodies against modified FGE polypeptides. The fragments should be short, between 5 and 20 amino acids, and preferably between 5 and 10 amino acids. Using known selection techniques, specific epitopes can be selected and used to generate monoclonal or polyclonal antibodies. Such antibodies have utility in the assaying protease resistant FGE activity, specifically identifying the expression of protease resistant FGE, and in the purification of the modified FGE from cell culture. The skilled person is well in the position to generate monoclonal or polyclonal antibodies or a fragment or derivatives such as F(ab), F(ab′), F(ab′)2, Fv, Fc, and single chain antibodies which are produced by recombinant DNA techniques or by enzymatic or chemical cleavage of intact antibodies (see, for example Antibodies: A Laboratory Manual, Harlow and Lane (eds), Cold Spring Harbor Press, (1988)), see, for example, U.S. Pat. Nos. RE 32,011, 4,902,614, 4,543,439, and 4,411,993, and Monoclonal Antibodies: A New Dimension in Biological Analysis, Plenum Press, Kennett, McKearn and Bechtol (eds.) (1980)). In a preferred embodiment the antibody selectively binds to the FGE variant of the invention.

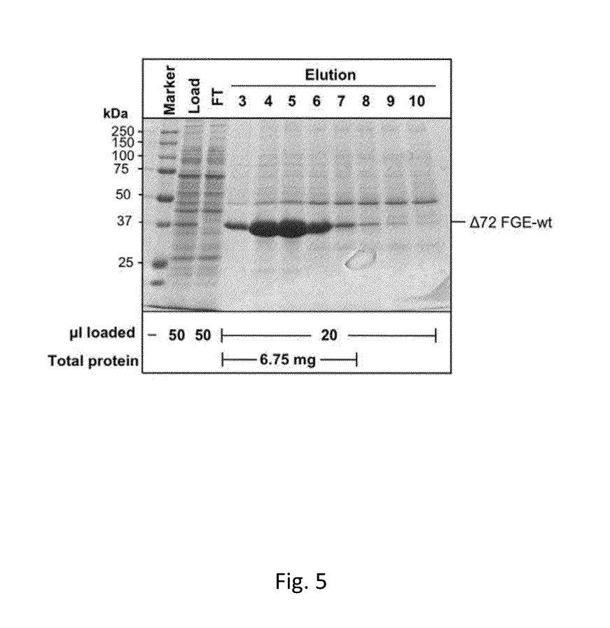

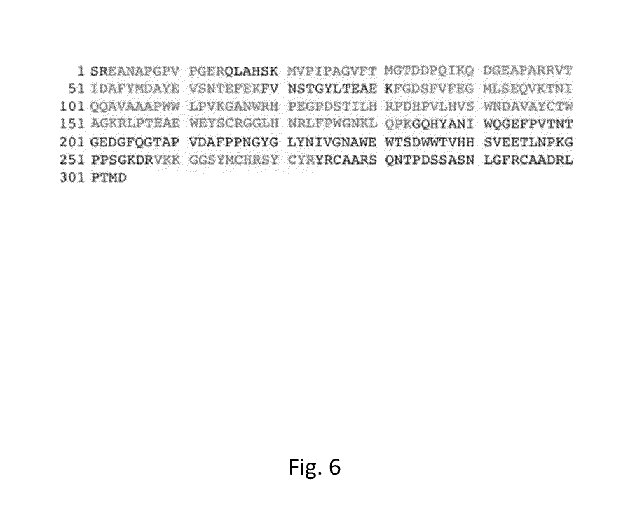

As stated above, insect cells can secrete the desired FGE polypeptide product into the cultivation medium and can subsequently be purified to a high degree of purity; see also Example 7, and FIGS. 5 and 6. The same holds true for mammalian cells as shown in Example 11. Thus, in a preferred embodiment the FGE polypeptide generated by the process or the FGE variants or fragment thereof are secreted into the medium.

As an alternative, various other mammalian cells can be used to express the FGE variants of the invention wherein the cells are optimized for high FGE variant protein expression such as fed-batch expression well known for the production of e.g. recombinant antibody generation. In addition, system can be used and the cells are cultivated under conditions wherein the proteases which recognize the claimed furin cleavage motif are not active. As stated above, this can be achieved by various ways; see supra.

As shown in Example 5 and 6 FGE variants can be purified by chromatographic means. Hence, in another aspect the present invention relates to a method of providing highly purified FGE or a functional variant or a fragment thereof, the method comprising the steps of (i) to (ii), optionally additional (ia) and (ib) of process of the invention using insect cells, wherein the polypeptides of the present invention are expressed. In one embodiment the above described vector is used wherein optionally a tag for purification is encoded by the vector and further comprising the steps of (ii) collecting the produced FGE polypeptide from the cell culture medium; iii) and purifying the produced FGE polypeptide by chromatographic means.

In this context, it is worth mentioning that the FGE polypeptides produced in insect cells have certain advantages over other cell culture systems. The insect cell system is the closest system to the mammalian cells. Recent reports have shown that insect cells can be grown without serum, so viruses and prions are no longer an issue. Insect cells can secrete the desired product so downstream processing is approximately the same as for yeast expression systems. If the cells are grown in serum free medium, approval gets much easier and the downstream process is much cheaper because of no additional steps to yield a higher level of pureness. The same is shown for mammalian expressed recombinant proteins, which are used to produce therapeutically active polypeptides. Thus, highly purified FGE variants and/or fragments thereof are suitable for use as a medicament in the treatment of a disease or condition or comprised in a diagnostic composition.

Useful derivatives of the modified polypeptides of the invention include, for example, modified human FGE polypeptides attached to at least one additional chemical moiety, or to at least one additional heterologous polypeptide to form covalent or aggregate conjugate such as glycosyl groups, lipids, phosphate, acetyl groups, or C-terminal or N-terminal fusion proteins and the like. In a preferred embodiment, the tag is encoded by the vector described above. Preferred heterologous polypeptides include those that facilitate purification, stability, cellular or tissue targeting, or secretion of the modified human FGE. Modifications of the amino acid sequence of human FGE polypeptides can be accomplished by any of a number of known techniques. For example, mutations can be introduced at particular locations by known procedures such as oligonucleotide-directed mutagenesis (Walder et al, 1986, Gene, 42:133; Bauer et al., 1985, Gene 37:13; Craik, 1985, BioTechniques, 12-19; Smith et al., 1981, Genetic Engineering: Principles and Methods, Plenum Press; and U.S. Pat. No. 4,518,584 and U.S. Pat. No. 4,737,462). The modified human FGE polypeptides of the present invention are preferably provided in an isolated form, and preferably are substantially purified. The polypeptides can be recovered and purified from recombinant cell cultures by known methods, including ammonium sulfate or ethanol precipitation, anion or cation exchange chromatography, phosphocellulose chromatography, hydrophobic interaction chromatography, affinity chromatography, hydroxylapatite chromatography, and lectin chromatography. In a preferred embodiment the FGE polypeptide is purified by chromatographic means comprising essentially consisting of anion exchange chromatography (AEX) reverse phase HPLC (RP-HPLC), hydroxyapatite, hydrophobic interaction (HIC), cation exchange (CEX), affinity (i.e. immunoaffinity or dye ligands) and size exclusion (gel filtration) (SEC) chromatography. Some intermediate steps are also common such as concentration, diafiltration, ultrafiltration, dialysis, precipitation with ethanol, salt and others.

Modified human FGE can be fused to heterologous regions used to facilitate purification of the polypeptide. Many of the available peptides (peptide tags) allow selective binding of the fusion protein to a binding partner. Non-limiting examples of peptide tags include 6-His, thioredoxin, hemaglutinin, GST, and the OmpA signal sequence tag. A binding partner that recognizes and binds to the peptide can be any molecule or compound including metal ions (for example, metal affinity columns), antibodies, antibody fragments, and any protein or peptide, which binds the heterologous peptide to permit purification of the fusion protein.

The purified affinity FGE polypeptide may be then attached to a suitable matrix such as agarose beads, acrylamide beads, glass beads, cellulose, various acrylic copolymers, hydroxylalkyl methacrylate gels, polyacrylic and polymethacrylic copolymers, nylon, neutral and ionic carriers, and the like. Attachment of the affinity FGE polypeptide to the matrix may be accomplished by methods such as those described in Methods in Enzymology, 44 (1976), or by other means known in the art. Attachment of the affinity FGE polypeptide to the matrix serves to immobilize the affinity FGE polypeptide. Such immobilized FGE polypeptide can be used to be incubated with another polypeptide of interest in order to allow the immobilized FGE to convert a suitable cysteine residue on the polypeptide of interest into a formylglycine amino acid.

Using molecular oxygen and an reducing agent, FGE oxidizes a cysteine residue in the substrate to an active site 3-oxoalanine residue, which is also called C(alpha)-formylglycine. Known substrates include for examples GALNS (SEQ ID NO: 109), ARSA (SEQ ID NO: 110), STS and ARSE (SEQ ID NO: 110), further substrates for examples any of the 17 human sulfatases are well known to the skilled person.

As shown in the Examples the present inventors for the first time successfully purified fl-FGE produced in insect cells. In contrast to Δ72-FGE, which needs DTT as a reducing agent for in vitro function, this fl-FGE variant is also active in the presence of the significantly milder reductant glutathione, which probably is the physiological co-substrate. Thus, fl-FGE-R69A/R72A purified from insect cells as described here opens up new avenues to in vitro applications including site-directed FGly-modification of proteins/peptides with the aim of downstream orthogonal aldehyde-mediated coupling reactions. The use of glutathione as reducing agent will be advantageous for many protein substrates, as physiological disulfide bridges should remain intact under these conditions.

Thus, in another aspect of the present application, the present invention relates to a method of producing an aldehyde tag in a polypeptide of interest, comprising the steps of (i) incubating a polypeptide of interest having a motif comprising a cysteine which can be processed by FGE in vitro, together with the FGE enzyme or a functional variant thereof of the invention or the purified FGE defined above in the presence of a reducing agent under conditions suitable for enzymatic activity to allow conversion of an amino acid residue to a formylglycine (FGIy) residue in the polypeptide and produces a converted tagged polypeptide; (ii) recovering the polypeptide with the newly generated tag. In a further embodiment, the method further comprises the step of (iii) attaching a moiety to the aldehyde of the newly generated formylglycine, i.e. coupling a moiety of interest to the newly generated tag of step (ii).

In other words, an in vitro method of producing a tag in a polypeptide of interest, comprising the steps of incubating a polypeptide having a motif comprising a heterologous sulfatase motif with the target cysteine residue, together with the FGE polypeptide or a functional variant thereof in the presence of a reducing agent, preferably glutathione (GSH) under conditions suitable for enzymatic activity of the inventive FGE polypeptide or functional variant thereof to allow conversion of an amino acid residue to a formylglycine (FGIy) residue in the polypeptide and produces a converted tagged polypeptide. As is evident from the above discussion of aldehyde tagged polypeptides, the modified heterologous sulfatase motif of the modified polypeptide can be positioned at any desired site of the polypeptide. Aldehyde tags can be positioned at any location within a target polypeptide at which it is desired to provide for conversion and/or modification of the target polypeptide, with the proviso that the site of the aldehyde tag is accessible for conversion by an FGE in its folded conformation and/or subsequent modification at the FGly.

Furthermore, the produced aldehyde tag can be further covalently coupled to a moiety of interest in order to produce an aldehyde moiety in a polypeptide of interest.

In accordance with the present invention the formylglycine (FGIy) is a converted tag. A tag is a site-specific labeling of a protein. The polypeptide of interest can be any polypeptide as long as it contains a moiety which can be recognized by the FGE protein produced by the present invention. Preferably, the FGE is a fl FGE variant of the present invention. In particular the polypeptide exhibits a moiety comprising or essentially consisting of a sequence from GALNS, ARSA, STS and ARSE naturally occurring or the sulfatase moiety described as “SUMF1-type” FGE in Cosma et al. Cell 2003, 113, (4), 445-56; Dierks et al. Cell 2003, 113, (4), 435-44 and WO2009/120611 or synthetic generated moiety such as LCTPSR, MCTPSR, VCTPSR, LCSPSR, LCAPSR, LCVPSR, and LCGPSR (SEQ ID NO: 47). Other specific sulfatase motifs are readily apparent from the US2013203111. Preferably, the cysteine residue (C) to be modulated is located in the expressed polypeptide in such a way that the FGE variant can oxidize the cysteine residue to generate formylglycine. By that an aldehyde functional group is generated. Subsequently the aldehyde group is further incubated with a partner which is reactive in order to attach a moiety of interest, i.e. a label. By that the polypeptide of interest will exhibit an aldehyde group covalently attached to a moiety of interest tag on a distinct pre-determined location of the polypeptide.

By “aldehyde tag” or “ald-tag” is meant an amino acid sequence that contains an amino acid sequence derived from a sulfatase motif which is capable of being converted, or which has been converted, by action of a formylglycine generating enzyme (FGE) to contain a Cα-formylglycine residue (referred to herein as “FGly”). The FGly residue generated by an FGE is often referred to in the literature as a “formylglycine”. Stated differently, the term “aldehyde tag” is used herein to refer to an amino acid sequence comprising an “unconverted” sulfatase motif (i.e., a sulfatase motif in which the cysteine residue has not been converted to FGly by an FGE, but is capable of being converted) as well as to an amino acid sequence comprising a “converted” sulfatase motif (i.e., a sulfatase motif in which the cysteine residue has been converted to FGly by action of an FGE).

By “conversion” as used in the context of action of a formylglycine generating enzyme (FGE) on a sulfatase motif refers to biochemical modification of a cysteine residue in a sulfatase motif to a formylglycine (FGly) residue (Cys to FGly). The present invention exploits a naturally-occurring, genetically-encodable sulfatase motif for use as a peptide tag, i.e, aldehyde tag, to direct site-specific modification of a polypeptide.

In US2013203111 suitable sulfatase motifs are described as well as the generation of aldehyde tags using FGE delta 72, i.e. the truncated bacterial version. The disclosure content of US2013203111 as well as WO 2009/120611 applications are incorporated herein by reference in its entirety. The aldehyde tagged, FGly-containing polypeptides can be subjected to modification to provide for attachment of a wide variety of moieties. Exemplary labels of interest include, but are not necessarily limited to, a detectable label, a small molecule, a peptide, and the like. In general, the label can provide for one or more of a wide variety of functions or features. Exemplary label moieties include detectable labels e.g., dye labels e.g., chromophores, fluorophores, biophysical probes spin labels, NMR probes, FRET-type labels e.g., at least one member of a FRET pair, including at least one member of a fluorophore/quencher pair, BRET-type labels e.g., at least one member of a BRET pair, immune-detectable tags e.g., FLAG, His(6), and the like, localization tags e.g., to identify association of a tagged polypeptide at the tissue or molecular cell level e.g., association with a tissue type, or particular cell membrane, and the like; light-activated dynamic moieties e.g., azobenzene mediated pore closing, azobenzene mediated structural changes, photodecaging recognition motifs; water soluble polymers e.g., PEGylation; purification tags e.g., to facilitate isolation by affinity chromatography e.g., attachment of a FLAG epitope; membrane localization domains e.g., lipids or GPI-type anchors; immobilization tags e.g., to facilitate attachment of the polypeptide to a surface, including selective attachment, and drugs e.g., to facilitate drug targeting, e.g., through attachment of the drug to an antibody; targeted delivery moieties, e.g., ligands for binding to a target receptor e.g., to facilitate viral attachment, attachment of a targeting protein present on a liposome, etc., and the like. The reactive partner for the aldehyde tagged polypeptide can comprise a small molecule drug, toxin, or other molecule for delivery to the cell and which can provide for a pharmacological activity or can serve as a target for delivery of other molecules.

The aldehyde moiety of a converted aldehyde tag can be used for a variety of applications including, but not limited to, visualization using fluorescence or epitope labeling (e.g., electron microscopy using gold particles equipped with aldehyde reactive groups), protein immobilization (e.g., protein microarray production), protein dynamics and localization studies and applications, and conjugation of proteins with a moiety of interest (e.g., moieties that improve a parent protein's therapeutic index (e.g., PEG), targeting moieties (e.g., to enhance bioavailability to a site of action), and biologically active moieties (e.g., a therapeutic moiety). The aldehyde tagged, FGly-containing polypeptides can be subjected to modification to provide for attachment of a wide variety of moieties.

The moiety of interest is provided as component of a reactive partner for reaction with an aldehyde of the FGly residue of a converted aldehyde tag of the tagged polypeptide. Since the methods of tagged polypeptide modification are compatible with conventional chemical processes, the methods of the invention can exploit a wide range of commercially available reagents to accomplish attachment of a moiety of interest to a FGly residue of an aldehyde tagged polypeptide. For example, aminooxy, hydrazide, hydrazine, or thiosemicarbazide derivatives of a number of moieties of interest are suitable reactive partners, and are readily available or can be generated using standard chemical methods.

For example, an aminooxy-PEG can be generated from monoamino-PEGs and aminooxyglycine using standard protocols. The aminooxy-PEG can then be reacted with a converted aldehyde tagged polypeptide to provide for attachment of the PEG moiety. Delivery of a biotin moiety to a converted aldehyde tagged polypeptide can be accomplished using aminooxy biotin, biotin hydrazide or 2,4 dinitrophenylhydrazine.

Provided the present disclosure, the ordinarily skilled artisan can readily adapt any of a variety of moieties to provide a reactive partner for conjugation to an aldehyde tagged polypeptide as contemplated herein.

In other embodiments, an aldehyde tag site is positioned at a site which is post-translationally modified in the native target polypeptide. For example, an aldehyde tag can be introduced at a site of glycosylation (e.g., N-glycosylation, O-glycosylation), phosphorylation, sulftation, ubiquitination, acylation, methylation, prenylation, hydroxylation, carboxylation, and the like in the native target polypeptide. Consensus sequences of a variety of post-translationally modified sites, and methods for identification of a post-translationally modified site in a polypeptide, are well known in the art. It is understood that the site of post-translational modification can be naturally-occurring or such a site of a polypeptide that has been engineered (e.g., through recombinant techniques) to include a post-translational modification site that is non-native to the polypeptide (e.g., as in a glycosylation site of a hyperglycosylated variant of EPO). In the latter embodiment, polypeptides that have a non-native post-translational modification site and which have been demonstrated to exhibit a biological activity of interest are of particular interest. The disclosure also provides herein methods for identifying suitable sites for modification of a target polypeptide to include an aldehyde tag. For example, one or more aldehyde tagged-target polypeptides constructs can be produced, and the constructs expressed in a cell expressing an FGE, or exposed to FGE following isolation from the cell (as described in more detail below). The aldehyde tagged-polypeptide can then be contacted with a reactive partner that, if the aldehyde tag is accessible, provides for attachment of a detectable moiety to the FGIy of the aldehyde tag. The presence or absence of the detectable moiety is then determined. If the detectable moiety is detected, then positioning of the aldehyde tag in the polypeptide was successful. In this manner, a library of constructs having an aldehyde tag positioned at different sites in the coding sequence of the target polypeptide can be produced and screened to facilitate identification of an optimal position of an aldehyde tag. In addition or alternatively, the aldehyde tagged-polypeptide can be tested for a biological activity normally associated with the target polypeptide, and/or the structure of the aldehyde tagged-polypeptide assessed (e.g., to assess whether an epitope normally present on an extracellular cell surface in the native target polypeptide is also present in the aldehyde tagged-polypeptide).

As more fully described in the Examples below, human wild type FGE protein (41 kDa+/−3 kDa) overexpressed and isolated from mammalian or insect cell cultures, when analyzed, for example, by electrophoresis, contains a number of polypeptides, shown as at least two bands on a gel. A prominent band in the mixture of proteins has a molecular weight of 37 kDa+/−3 kDa as measured by SDS PAGE, thus, evidencing that degradation of human FGE resulted from cleavage at the furin cleavage site or next to it. Such truncation of human FGE produces a shortened polypeptide wherein the N-terminal conserved cysteine residues are removed that are thought to be involved in interacting with glutathione. Accordingly, cleavage of FGE at the furin cleavage site (or next to it) is thought to remove a portion of the molecule that is required for biological activity.

In one embodiment, the present invention provides for the first time eukaryotic FGE polypeptide variants which comprise the full length sequence of activated FGE which can use glutathione in the generation of formlyglycine reaction. Further suitable reducing agents are reduced glutathione (GSH), dithiothreitol (DTT), dithioerythritol (DTE), cysteine, β-mercaptoethanol, wherein advantageously according to the teaching of the present invention glutathione is used as a reducing agent.

In line with the teaching of the invention, polypeptides of interest can be site directly labeled, or in vitro oxidized at a specific cysteine residue under mild conditions since: glutathione is the component of the physiological redox system (GSH/GSSG). Thus avoiding that denaturation i.e. un- or misfolding of the polypeptides due to the harsh reduction conditions (e.g. use of DTT). Once a polypeptide had been denatured, i.e. unfolded, it is required to refold the polypeptide in order to retain its biologically active state. Furthermore, the refolding process is time consuming and it is difficult to obtain standardized refolding parameters. Finally, removal of the denaturant including a filtration step leads to a loss of total protein yield. This is particularly true for diagnostic or therapeutic recombinantly expressed polypeptides exhibiting at least one cysteine disulfide bridge. Thus, in a preferred embodiment, the polypeptide of interest comprises at least one disulfide bridge.

In one embodiment, the aldehyde tag-based methods of protein modification are applied to modification of polypeptides that may provide for a therapeutic benefit, particularly those polypeptides for which attachment to a moiety can provide for one or more of, for example, an increase in serum half-life, a decrease in an adverse immune response, additional or alternate biological activity or functionality, and the like or other benefit or reduction of an adverse side effect. Where the therapeutic polypeptide is an antigen for a vaccine, modification can provide for an enhanced immunogenicity of the polypeptide.

Examples of classes of a polypeptide of interests are therapeutic proteins including those that are cytokines, chemokines, growth factors, hormones, antibodies, and antigens. Further examples include erythropoietin (EPO, e.g., native EPO, synthetic EPO (see, e.g., US 2003/0191291), human growth hormone (hGH), bovine growth hormone (bGH), follicle stimulating hormone (FSH), interferon (e.g., IFN-gamma, IFN-beta, IFN-alpha, IFN-omega, consensus interferon, and the like), insulin, insulin-like growth factor (e.g., IGF-I, IGF-II), blood factors (e.g., Factor VIII, Factor IX, Factor X, tissue plasminogen activator (TPA), and the like), colony stimulating factors (e.g., granulocyte-CSF (G-CSF), macrophage-CSF (M-CSF), granulocyte-macrophage-CSF (GM-CSF), and the like), transforming growth factors (e.g., TGF-beta, TGF-alpha), interleukins (e.g., IL-1, IL-2, IL-3, IL-4, IL-5, IL-6, IL-7, IL-8, IL-12, and the like), epidermal growth factor (EGF), platelet-derived growth factor (PDGF), fibroblast growth factors (FGFs, e.g., aFGF, bFGF), glial cell line-derived growth factor (GDNF), nerve growth factor (NGF), RANTES, and the like.

Further examples include antibodies, e.g., polyclonal antibodies, monoclonal antibodies, humanized antibodies, antigen-binding fragments (e.g., F(ab)′, Fab, Fv), single chain antibodies, and the like. Of particular interest are antibodies that specifically bind to a tumor antigen, an immune cell antigen (e.g., CD4, CD8, and the like), an antigen of a microorganism, particularly a pathogenic microorganism (e.g., a bacterial, viral, fungal, or parasitic antigen), and the like.

Moreover, the present invention relates to compositions comprising the aforementioned FGE variants or fragments thereof, such as the peptides comprising the mutated N-terminal furin-cleavage motif of the invention or chemical derivatives thereof, or the polynucleotide, vector or cell or further comprising the tagged polypeptide generated by the method of the invention.

In a further embodiment, the aldehyde-tagged polypeptides and/or the aldehyde-tagged polypeptides attached to at least one further moiety of interests, produced with the help of the inventive fl FGE variants are comprised within said compositions.

The invention provides compositions containing a substantially purified modified FGE polypeptide of the invention and a carrier. For therapeutic applications, the invention provides compositions adapted for pharmaceutical use, for example, containing a pharmaceutically acceptable carrier. Pharmaceutical compositions of the invention are administered to cells, tissues, or patients. The pharmaceutical compositions containing a FGE-modified polypeptide are also useful as vaccine adjuvants, for example, useful for obtaining long-term immunity.

The composition of the present invention may further comprise a pharmaceutically acceptable carrier. The term “chemical derivative” describes a molecule that contains additional chemical moieties that are not normally a part of the basic molecule. Such moieties may improve the solubility, half-life, absorption, etc. of the basic molecule. Alternatively the moieties may attenuate undesirable side effects of the basic molecule or decrease the toxicity of the basic molecule. Examples of such moieties are described in a variety of texts, such as Remington's Pharmaceutical Sciences.

In a further preferred embodiment, that relates to an aqueous, buffered pharmaceutical composition comprising at least one FGE variant, preferably at least one fl FGE variant and a buffer, wherein the buffer comprises imidazole, preferably 200 to 300 mM imidazole, more preferably 250 mM, wherein the composition exhibits long term stability. As shown in Example 19, the presence of 250 mM imidazole maintains the protein stability upon (long-term) storage of purified recombinant fl-FGE-R69A/R72A even after freezing and thawing cycles.

While the various compositions or polypeptides described herein may be shown with no protecting groups, in certain embodiments (e.g., particularly for oral administration), they can bear one, two, three, four, or more protecting groups. The protecting groups can be coupled to the C- and/or N-terminus of the peptide(s) and/or to one or more internal residues comprising the peptide(s) (e.g., one or more R-groups on the constituent amino acids can be blocked). Thus, for example, in certain embodiments, any of the peptides described herein can bear, e.g., an acetyl group protecting the amino terminus and/or an amide group protecting the carboxyl terminus, such as “Ac-RYSR-NH₂” (SEQ ID NO: 48 with blocking groups) or any other of the above mentioned modified furin cleavage motif amino acid sequences, either or both of these protecting groups can be eliminated and/or substituted with another protecting group. These amino and/or carboxyl termini of the subject peptides of this invention can improve oral delivery and can also increase serum half-life as described in WO2009/032693. Suitable and further protecting/blocking groups are well known to those of skill as are methods of coupling such groups to the appropriate residue(s) comprising the peptides of this invention (see, e.g., Greene et al., (1991) Protective Groups in Organic Synthesis, 2^{nd} ed., John Wiley & Sons, Inc. Somerset, N.J.). For further reading regarding fusing the peptides or polypeptides of the invention together with a linker region or with other peptides see WO2009/032693, which is hereby incorporated by reference in its entirety for its teaching of specific linked peptides and administration and formulation of peptides as a medicine.

Examples of suitable pharmaceutical carriers are well known in the art and include phosphate buffered saline solutions, water, emulsions, such as oil/water emulsions, various types of wetting agents, sterile solutions etc. Compositions comprising such carriers can be formulated by well known conventional methods. These pharmaceutical compositions can be administered to the subject at a suitable dose. Administration of the suitable compositions may be effected by different ways, e.g., by intravenous, intraperitoneal, subcutaneous, intramuscular, topical or oral, intrahecal, intradermal administration. Aerosol formulations such as nasal spray formulations include purified aqueous or other solutions of the active agent with preservative agents and isotonic agents. Such formulations are preferably adjusted to a pH and isotonic state compatible with the nasal mucous membranes. Formulations for rectal or vaginal administration may be presented as a suppository with a suitable carrier.

The dosage regimen will be determined by the attending physician and clinical factors. As is well known in the medical arts, dosages for any one patient depends upon many factors, including the patient's size, body surface area, age, the particular compound to be administered, sex, time and route of administration, general health, and other drugs being administered concurrently. A typical dose can be, for example, in the range of 0.001 μg to 10 mg (or of nucleic acid for expression or for inhibition of expression in this range); however, doses below or above this exemplary range are envisioned, especially considering the aforementioned factors. Generally, the regimen as a regular administration of the pharmaceutical composition should be in the range of 0.01 μg to 10 mg units per day. If the regimen is a continuous infusion, it should also be in the range of 0.01 μg to 10 mg units per kilogram of body weight per minute, respectively. Progress can be monitored by periodic assessment. Dosages will vary but a preferred dosage for intravenous administration of DNA is from approximately 10⁶ to 10¹² copies of the DNA molecule.

The compositions of the invention may be administered locally or systemically. Administration will generally be parenterally, e.g., intravenously; DNA may also be administered directly to the target site, e.g., by biolistic delivery to an internal or external target site or by catheter to a site in an artery. Preparations for parenteral administration include sterile aqueous or non-aqueous solutions, suspensions, and emulsions. Examples of non-aqueous solvents are propylene glycol, polyethylene glycol, vegetable oils such as olive oil, and injectable organic esters such as ethyl oleate. Aqueous carriers include water, alcoholic/aqueous solutions, emulsions or suspensions, including saline and buffered media. Parenteral vehicles include sodium chloride solution, Ringer's dextrose, dextrose and sodium chloride, lactated Ringer's, or fixed oils. Intravenous vehicles include fluid and nutrient replenishers, electrolyte replenishers (such as those based on Ringer's dextrose), and the like. Preservatives and other additives may also be present such as, for example, antimicrobials, anti-oxidants, chelating agents, and inert gases and the like. Furthermore, the pharmaceutical composition of the invention may comprise further agents such as interleukins or interferons depending on the intended use of the pharmaceutical composition.

Therapeutic or diagnostic compositions of the invention are administered to an individual in an effective dose sufficient to treat or diagnose disorders in which modulation of FGE-related activity is indicated. The effective amount may vary according to a variety of factors such as the individual's condition, weight, sex and age. Other factors include the mode of administration. The pharmaceutical compositions may be provided to the individual by a variety of routes such as by oral, intrahecal, intracoronary, intraperitoneal, subcutaneous, intravenous, transdermal, intrasynovial, intramuscular or oral routes. In addition, co-administration or sequential administration of other agents may be desirable.

A therapeutically effective dose refers to that amount of FGE variant, a fragment thereof, polynucleotides and vectors of the invention ameliorate the symptoms or condition. Therapeutic efficacy and toxicity of such compounds can be determined by standard pharmaceutical procedures in cell cultures or experimental animals, e.g., ED50 (the dose therapeutically effective in 50% of the population) and LD50 (the dose lethal to 50% of the population). The dose ratio between therapeutic and toxic effects is the therapeutic index, and it can be expressed as the ratio, LD50/ED50. Thus; in a preferred embodiment, the composition of the present invention is either a pharmaceutical composition and further comprises a pharmaceutically acceptable carrier, or a diagnostic composition and optionally comprises reagents conventionally used in immune- or nucleic acid based diagnostic methods.

Stieneke-Grober, A, et al. (1992) EMBO J. 11, 2407-2414 have shown that acylated peptidyl chloromethanes (—CH₂Cl; ‘chloromethylketones’) containing a consensus furin cleavage sequence, such as decanoyl-Arg-Glu-Lys-Arg-CH₂Cl, inhibit cleavage of influenza-virus HA by furin in vitro as well as in vivo as influenza HA, HIV gp160, cytomegalovirus glycoprotein B and parainfluenza-virus glycoprotein F₀, thereby inhibiting formation of infectious viruses Vey, M., et al., (1995) Virology 206, 746-749 or Ortmann, D. et al., (1994) J. Virol. 68, 2772-2776. Thus, without intending to be bound by theory, the FGE polypeptide or fragment thereof as defined above, can be used as a medicament in order to treat, diagnose or prevent virus replication in a subject by use of the present invention. In particular, use of the a peptide of the invention which exhibit an amino acid exchange wherein the exchange leads to an improved binding to protease but will not be cleaved by the protease will result in a complete blocking of the furin protease and thus will inhibit cleavage of virus polypeptide by furin and subsequently infection.

Furthermore, Multiple sulfatase deficiency (MSD) is a rare inborn error of metabolism affecting posttranslational activation of sulfatases by the formylglycine generating enzyme (FGE). Due to mutations in the encoding SUMF1 gene, FGE's catalytic capacity is impaired resulting in reduced cellular sulfatase activities. Thus, using any of the FGE variants having impaired furin cleavage motif would be beneficial for treatment since only small amounts of FGE variants are required since these variants exhibit full biological activity. For use and generation of FGE variants in treatment of MSD and related diseases see for further reading WO2004072275.

According to a further aspect of the invention, a method of treating Multiple Sulfatase Deficiency, is provided. The method involves administering to a subject in need of such treatment an agent that modulates Cα-formylglycine generating activity, in an amount effective to treat Multiple Sulfatase Deficiency in the subject.

In a further aspect, the invention relates to a method of treating a subject suffering from Morquio A syndrome, Multiple Sulfatase Deficiency (MSD) or a FGE deficiency related disease or condition comprising administering an effective amount of a pharmaceutical or diagnostic composition of the invention comprising the FGE variant or a fragment thereof and a pharmaceutical acceptable carrier to the subject. Variant polypeptide substrates having increased or decreased affinity for enzymes compared to their endogenous homologues are useful as therapeutic agonists and antagonists as well as for diagnostics.

In certain embodiments, the sulfatase deficiency includes, but is not limited to, Metachromatic leukodystrophy(MLD), Maroteaux-Lamy-Syndrome/MPS VI, X-linked Ichthyose (XLI), -linked Recessive Chondrodysplasia Punctata 1, Chondrodysplasia Punctata (CDPX1), Sanfilippo D/MPS IIID or Hunter-Syndrome/MPSII.

From the scientific literature it is known that furin is also involved in tumor metastasis, activation and virulence of many bacterial and viral pathogens and in neurodegenerative processes associated with Alzheimer's disease. Proteolytic activation of envelope glycoproteins is necessary for the entry of viruses into host cells and, hence, for their ability to undergo multiple replication cycles. In some cases, it has also been shown that the cleavability of the envelope glycoproteins is an important determinant for viral pathogenicity. The haemagglutinins (HAs) of mammalian influenza viruses and av irulent avian-influenza viruses, which cause local infection, are susceptible to proteolytic cleavage only in limited cell types, such as those of the respiratory and alimentary tracts. In contrast, those of virulent avian-influenza viruses caused a systemic infection see for further reading Klenk, H.-D. and Rott, R. (1988) Adv. Virus Res. 34, 247-281. As outlined in detail in U.S. Pat. No. 7,033,991 small, polybasic peptides such as hexa- to non a-peptides having L-Arg or L-Lys in most positions are effective as furin inhibitors. Removing the peptide terminating groups can improve inhibition of furin. High inhibition was seen in a series of non-amidated and non-acetylated polyarginines. The most potent inhibitor identified to date, non a-L-arginine, had a Ki against furin of 40 nM. Non-acetylated, poly-D-arginine-derived molecules are preferred furin inhibitors for therapeutic uses, such as inhibiting certain bacterial infections, viral infections, and cancers.

Thus, in another aspect of the present invention, the polypeptides derived from FGE comprising the amino acid 63 to 82, preferably 69 to 72 alone or coupled to another functional group or moiety and relating to the group of peptides which decrease the furin cleavage activity at least in vitro can be used as a medicament to treat or prevent virus infection or replication in a subject.

Naturally the present invention relates in another aspect to a polypeptide conjugate obtained from the method of the invention designed to be administered as a medicament or a vaccine.

In a further aspect, the invention relates to a kit comprising the polypeptide and/or the host cell and/or insect cell of the invention and/or the polynucleotide obtained by the process and/or the polypeptide variants and/or the primer and/or the vector and/or any combination thereof optionally with reagents and/or instructions for use or any combination thereof. The kit alternatively comprises a package containing an agent that selectively binds to any of the foregoing FGE isolated nucleic acids, or expression products thereof, and a control for comparing to a measured value of binding of said agent any of the foregoing FGE isolated nucleic acids or expression products thereof.

The inventive FGE polypeptide variants attached to a suitable matrix, as described above, are particularly useful in a kit for diagnostic and/or therapeutic purposes, since FGE is directly ready for use. As shown in Example 19, fl FGE variants produced by the present process are capable of also forming dimeric structures, which are more stable than the monomeric fl FGE variant forms, exhibit a different pH optimum compared to monomeric FGE while still being capable of using GSH as a reducing agent. Thereby, the present invention provides for the first time various fl FGE variants, which can be selected for different needs of an assay or application. In a further preferred embodiment, the kit comprises imidazole. As shown in Example 19, fl FGE variants of the present invention are formulated in a buffer containing imidazole (up to 250 mM) which increase the stability of the fl FGE variants dramatically.

In addition, the kit can contain instructions for using the components of the kit, particularly the compositions of the invention that are contained in the kit.

These and other embodiments are disclosed and encompassed by the description and examples of the present invention. Further literature concerning any one of the materials, methods, uses and compounds to be employed in accordance with the present invention may be retrieved from public libraries and databases, using for example electronic devices. For example the public database “Medline” may be utilized, which is hosted by the National Center for Biotechnology Information and/or the National Library of Medicine at the National Institutes of Health. Further databases and web addresses, such as those of the European Bioinformatics Institute (EBI), which is part of the European Molecular Biology Laboratory (EMBL) are known to the person skilled in the art and can also be obtained using internet search engines. An overview of patent information in biotechnology and a survey of relevant sources of patent information useful for retrospective searching and for current awareness is given in Berks, TIBTECH 12 (1994), 352-364.

The above disclosure generally describes the present invention. A more complete understanding can be obtained by reference to the following specific examples which are provided herein for purposes of illustration only and are not intended to limit the scope of the invention. Several documents are cited throughout the text of this specification. Full bibliographic citations may be found at the end of the specification immediately preceding the claims. The contents of all cited references (including literature references, issued patents, published patent applications as cited throughout this application and manufacturer's specifications, instructions, etc) are hereby expressly incorporated by reference; however, there is no admission that any document cited is indeed prior art as to the present invention.

### EXAMPLES

The examples which follow further illustrate the invention, but should not be construed to limit the scope of the invention in any way. Detailed descriptions of conventional methods, such as those employed in the construction of vectors and plasmids, the insertion of genes encoding polypeptides into such vectors and plasmids, the introduction of plasmids into host cells, and the expression and determination thereof of genes and gene products can be obtained from numerous publication, including Sambrook et al., (1989) Molecular Cloning: A Laboratory Manual, 2nd ed., Cold Spring Harbor Laboratory Press; “Molecular Biology Techniques Manual” 3rd Ed. ed by Coyne, James, Reid and Rybicki (2003) available at http://www.uct.ac.za/microbiology/manual/MolBiolManual.htm; “Molecular Biology Techniques: An Intensive Laboratory Course” by Ream, Field, and Field (Harcourt Brace & Company: 1998); “The Merck Manual of Diagnosis and Therapy” Seventeenth Ed. ed by Beers and Berkow (Merck & Co., Inc. 2003). Standard text books dedicated to baculovirus based expression systems are i.e.: Baculovirus and Insect Cell Expression Protocols, by Murahmmer, David W., ISBN: 9781588295378, 2007, Publisher: Springer Verlag. Baculovirus expression vectors, O'Reilly, D. R., Miller, L. K and Luckow, V. A., ISBN 0195091310, 1994, Oxford Univ. Press.

### Example 1

### Cloning of fl-FGE, fl-FGE-R69A/R72A and Δ72-FGE in pAcGP67 vector

1.1 Transfer vectors used for cloning FGE
a) pAcGP67-B-His7—used for cloning Δ72-FGE
b) pAcGP67-B-AlKa—used for cloning fl-FGE and fl-FGE-R69A/R72A
1.2 Description of pAcGP67-B-His7 vector pAcGP67-B-His7 plasmid is a kind gift from Dr. Santosh Lakshmi Gande (University of Frankfurt, Germany). This plasmid was generated by modification of the pAcGP67-B plasmid (BD Biosciences) to facilitate the expression and purification of C-terminally RGS-His7 tagged recombinant proteins in a baculovirus expression system. The cDNA sequence that encodes RGS-His7 followed by two stop codons, was cloned in frame between 5′-BamHI and 3′-PstI restriction sites in the MCS of pAcGP67-B.
1.3 Description of pAcGP67-B-AlKa Vector

fl-FGE expressed and purified from pAcGP67-B-His7 vector will carry 9 extra amino acid residues, encoded by the linker sequence, at the N-terminus, which is undesirable. To circumvent this problem, the linker sequence between the signal peptide and expressed protein of interest was shortened so that the purified protein will contain just 4 extra amino acids at the N-terminus after signal peptide cleavage. To achieve this, the DNA sequence between the EcoRV and EcoRI sites in pAcGP67-B-His7 (nucleotides 3998 to 4273) was exchanged in-frame with a DNA sequence lacking codons for 5 amino acid residues in the linker sequence. The (5′-EcoRV)-exchange sequence-(EcoRI-3′) was generated by PCR amplification using primerspAcGP67-EcoRV-F and pAcGP67-EcoRI-R and pAcGP67-B-His7 as template. In the resulting vector, named pAcGP67-B-AlKa, the BamHI and NcoI sites are lost as compared to the MCS of pAcGP67-B-His7. The presence of base pairs that code for Ala and Asp in the signal peptide cleavage site was maintained to preserve the cleavage site specificity. The cDNA sequence for full length FGE can be cloned between 5′-EcoRI and 3′-NotI sites inpAcGP67-B-AlKa.

### Primers for Generating “Exchange Sequence”:

a) pAcGP67-EcoRV-F (forward): (SEQ ID NO: 33)
5′-CGGATATCATGGAGATAATTAAA-3′
b) pAcGP67-EcoRI-R (reverse) (SEQ ID NO: 34):
5′-CCGGAATTCATCCGCCGCAAAGGCAGAATG-3′

1.4 Cloning of fl-FGE, fl-FGE-R69A/R72A and Δ72-FGE cDNAs into pAcGP67B-AlKa
1.4.1 fl-FGE (wild type, WT)

The cDNA sequence that encodes the mature human FGE (lacking the ER targeting signal sequence) was amplified by PCR with pBI-FGE-HA (Mariappan et al 2008) as template and the following primers:

a) pAC-FGE-Eco-F (forward primer)
(SEQ ID NO: 35):
5′-CCGGAATTCAGCCAGGAGGCCGGGACC-3′
b) BTVd72FGE-Not-R (reverse primer)
(SEQ ID NO: 36):
5′-ATAATGCGGCCGCTGTCCATAGTGGGCAGGCG-3′

The PCR product was purified and digested with EcoRI and NotI restriction enzymes and cloned in-frame into the 5′-EcoRI and 3′-NotI sites in the MCS of pAcGP67B-AlKa and verified by sequencing.

For sequencing, the following primers were used:

pAcGP67-forward (SEQ ID NO: 37):
5′ CCG GAT TAT TCA TAC CGT CCC 3′
pAcGP67-reverse (SEQ ID NO: 38):
5′ CGT GTC GGG TTT AAC ATT ACG 3′

cDNA-Sequence SEQ ID NO:1 (5′→3′) of fl-FGE:

GCGGATGAATTCAGCCAGGAGGCCGGGACCGGTGCGGGCGCGGGGTCCC
TTGCGGGTTCTTGCGGCTGCGGCACGCCCCAGCGGCCTGGCGCCCATGG
CAGTTCGGCAGCCGCTCACCGATACTCGCGGGAGGCTAACGCTCCGGGC
CCCGTACCCGGAGAGCGGCAACTCGCGCACTCAAAGATGGTCCCCATCC
CTGCTGGAGTATTTACAATGGGCACAGATGATCCTCAGATAAAGCAGGA
TGGGGAAGCACCTGCGAGGAGAGTTACTATTGATGCCTTTTACATGGAT
GCCTATGAAGTCAGTAATACTGAATTTGAGAAGTTTGTGAACTCAACTG
GCTATTTGACAGAGGCTGAGAAGTTTGGCGACTCCTTTGTCTTTGAAGG
CATGTTGAGTGAGCAAGTGAAGACCAATATTCAACAGGCAGTTGCAGCT
GCTCCCTGGTGGTTACCTGTGAAAGGCGCTAACTGGAGACACCCAGAAG
GGCCTGACTCTACTATTCTGCACAGGCCGGATCATCCAGTTCTCCATGT
GTCCTGGAATGATGCGGTTGCCTACTGCACTTGGGCAGGGAAGCGGCTG
CCCACGGAAGCTGAGTGGGAATACAGCTGTCGAGGAGGCCTGCATAATA
GACTTTTCCCCTGGGGCAACAAACTGCAGCCCAAAGGCCAGCATTATGC
CAACATTTGGCAGGGCGAGTTTCCGGTGACCAACACTGGTGAGGATGGC
TTCCAAGGAACTGCGCCTGTTGATGCCTTCCCTCCCAATGGTTATGGCT
TATACAACATAGTGGGGAACGCATGGGAATGGACTTCAGACTGGTGGAC
TGTTCATCATTCTGTTGAAGAAACGCTTAACCCAAAAGGTCCCCCTTCT
GGGAAAGACCGAGTGAAGAAAGGTGGATCCTACATGTGCCATAGGTCTT
ATTGTTACAGGTATCGCTGTGCTGCTCGGAGCCAGAACACACCTGATAG
CTCTGCTTCGAATCTGGGATTCCGCTGTGCAGCCGACCGCCTGCCCACT
ATGGACAGCGGCCGCGGAAGCCATCACCATCACCATCACCATTAA

Amino Acid Sequence of fl-FGE (SEQ ID NO:2):

ADEFSQEAGTGAGAGSLAGSCGCGTPQRPGAHGSSAAAHRYSREANAPG
PVPGERQLAHSKMVPIPAGVFTMGTDDPQIKQDGEAPARRVTIDAFYMD
AYEVSNTEFEKFVNSTGYLTEAEKFGDSFVFEGMLSEQVKTNIQQAVAA
APWWLPVKGANWRHPEGPDSTILHRPDHPVLHVSWNDAVAYCTWAGKRL
PTEAEWEYSCRGGLHNRLFPWGNKLQPKGQHYANIWQGEFPVTNTGEDG
FQGTAPVDAFPPNGYGLYNIVGNAWEWTSDWWTVHHSVEETLNPKGPPS
GKDRVKKGGSYMCHRSYCYRYRCAARSQNTPDSSASNLGFRCAADRLPT
MDSGRGSHHHHHHH*

1.4.2 fl-FGE-R69A/R72A

cDNA encoding FGE-R69A/R72A was generated by site directed mutagenesis PCR using pBI-FGE-HA (Mariappan et al. 2008) as template and mutagenesis primers. The resulting expression construct pBI-FGE-R69A/R72A-HA was verified for the presence of desired mutations (Arginine-69 and Arginine-72 converted to Alanine) by sequencing.

### Mutagenesis Primer Sequence:

FGE-R69AR72A:
(SEQ ID NO: 27)
5′-GCTCACGCATACTCGGCGGAGGCTAACGCTCCG-3′

The cDNA of fl-FGE-R69A/R72A was amplified by PCR using pBI-FGE-R69A/R72A-HA as template and primers pAc-FGE-Eco-F and BTVd72FGE-Not-R and cloned into pAcGP67B-AlKa as shown for fl-FGE-wt (see section 1.4.1).

Given below are the expected sequences of the mature secreted fl-FGE-R69A/R72A. cDNA sequence (5′→3′) (SEQ ID NO:3):

GCGGATGAATTCAGCCAGGAGGCCGGGACCGGTGCGGGCGCGGGGTCCCT
TGCGGGTTCTTGCGGCTGCGGCACGCCCCAGCGGCCTGGCGCCCATGGCA
GTTCGGCAGCCGCTCACGCATACTCGGCGGAGGCTAACGCTCCGGGCCCC
GTACCCGGAGAGCGGCAACTCGCGCACTCAAAGATGGTCCCCATCCCTGC
TGGAGTATTTACAATGGGCACAGATGATCCTCAGATAAAGCAGGATGGGG
AAGCACCTGCGAGGAGAGTTACTATTGATGCCTTTTACATGGATGCCTAT
GAAGTCAGTAATACTGAATTTGAGAAGTTTGTGAACTCAACTGGCTATTT
GACAGAGGCTGAGAAGTTTGGCGACTCCTTTGTCTTTGAAGGCATGTTGA
GTGAGCAAGTGAAGACCAATATTCAACAGGCAGTTGCAGCTGCTCCCTGG
TGGTTACCTGTGAAAGGCGCTAACTGGAGACACCCAGAAGGGCCTGACTC
TACTATTCTGCACAGGCCGGATCATCCAGTTCTCCATGTGTCCTGGAATG
ATGCGGTTGCCTACTGCACTTGGGCAGGGAAGCGGCTGCCCACGGAAGCT
GAGTGGGAATACAGCTGTCGAGGAGGCCTGCATAATAGACTTTTCCCCTG
GGGCAACAAACTGCAGCCCAAAGGCCAGCATTATGCCAACATTTGGCAGG
GCGAGTTTCCGGTGACCAACACTGGTGAGGATGGCTTCCAAGGAACTGCG
CCTGTTGATGCCTTCCCTCCCAATGGTTATGGCTTATACAACATAGTGGG
GAACGCATGGGAATGGACTTCAGACTGGTGGACTGTTCATCATTCTGTTG
AAGAAACGCTTAACCCAAAAGGTCCCCCTTCTGGGAAAGACCGAGTGAAG
AAAGGTGGATCCTACATGTGCCATAGGTCTTATTGTTACAGGTATCGCTG
TGCTGCTCGGAGCCAGAACACACCTGATAGCTCTGCTTCGAATCTGGGAT
TCCGCTGTGCAGCCGACCGCCTGCCCACTATGGACAGCGGCCGCGGAAGC
CATCACCATCACCATCACCATTAA

### Amino Acid Sequence (SEQ ID NO:4):

ADEFSQEAGTGAGAGSLAGSCGCGTPQRPGAHGSSAAAHAYSAEANAPGPVPGERQ
LAHSKMVPIPAGVFTMGTDDPQIKQDGEAPARRVTIDAFYMDAYEVSNTEFEKFVNS
TGYLTEAEKFGDSFVFEGMLSEQVKTNIQQAVAAAPWWLPVKGANWRHPEGPDSTI
LHRPDHPVLHVSWNDAVAYCTWAGKRLPTEAEWEYSCRGGLHNRLFPWGNKLQPK
GQHYANIWQGEFPVTNTGEDGFQGTAPVDAFPPNGYGLYNIVGNAWEWTSDWWTV
HHSVEETLNPKGPPSGKDRVKKGGSYMCHRSYCYRYRCAARSQNTPDSSASNLGFR
CAADRLPTMDSGRGSHHHHHHH*

### 1.4.3 Δ72-FGE

The cDNA sequence that encodes human FGE that lacks the N-terminal domain (amino acid residues 1-72) was amplified by PCR with pBI-FGE-HA (Mariappan et al 2008) as template and the following primers:

### Primers:

a) BTVd72FGE-Eco-F (Forward primer):
(SEQ ID NO: 39)
5′-CCGGAATTCGAGGCTAACGCTCCGGGC-3′
b) BTVd72FGE-Not-R (Reverse primer):
(SEQ ID NO: 40)
5′-ATAATGCGGCCGCTGTCCATAGTGGGCAGGCG-3′

The purified PCR product was digested with EcoRI and NotI restriction enzymes and cloned in-frame into the 5′-EcoRI and 3′-NotI sites in the MCS of pAcGP67B-His7 and verified by sequencing using the following primers.

pAcGP67-forward:
(SEQ ID NO: 41)
5′-CCGGATTATTCATACCGTCCC-3′
pAcGP67-reverse:
(SEQ ID NO: 42)
5′-CGTGTCGGGTTTAACATTACG-3′
ERp40-505c:
(SEQ ID NO: 43)
5′-GAGTGAGCAAGTGAAGAC-3′

Given below are the expected sequences of the mature secreted Δ72-FGE:

cDNA-Sequence (5′→3′) (SEQ ID NO:5):

GCGGATCTTGGATCCTCCATGGAATTCGAGGCTAACGCTCCGGGCCCCGT
ACCCGGAGAGCGGCAACTCGCGCACTCAAAGATGGTCCCCATCCCTGCTG
GAGTATTTACAATGGGCACAGATGATCCTCAGATAAAGCAGGATGGGGAA
GCACCTGCGAGGAGAGTTACTATTGATGCCTTTTACATGGATGCCTATGA
AGTCAGTAATACTGAATTTGAGAAGTTTGTGAACTCAACTGGCTATTTGA
CAGAGGCTGAGAAGTTTGGCGACTCCTTTGTCTTTGAAGGCATGTTGAGT
GAGCAAGTGAAGACCAATATTCAACAGGCAGTTGCAGCTGCTCCCTGGTG
GTTACCTGTGAAAGGCGCTAACTGGAGACACCCAGAAGGGCCTGACTCTA
CTATTCTGCACAGGCCGGATCATCCAGTTCTCCATGTGTCCTGGAATGAT
GCGGTTGCCTACTGCACTTGGGCAGGGAAGCGGCTGCCCACGGAAGCTGA
GTGGGAATACAGCTGTCGAGGAGGCCTGCATAATAGACTTTTCCCCTGGG
GCAACAAACTGCAGCCCAAAGGCCAGCATTATGCCAACATTTGGCAGGGC
GAGTTTCCGGTGACCAACACTGGTGAGGATGGCTTCCAAGGAACTGCGCC
TGTTGATGCCTTCCCTCCCAATGGTTATGGCTTATACAACATAGTGGGGA
ACGCATGGGAATGGACTTCAGACTGGTGGACTGTTCATCATTCTGTTGAA
GAAACGCTTAACCCAAAAGGTCCCCCTTCTGGGAAAGACCGAGTGAAGAA
AGGTGGATCCTACATGTGCCATAGGTCTTATTGTTACAGGTATCGCTGTG
CTGCTCGGAGCCAGAACACACCTGATAGCTCTGCTTCGAATCTGGGATTC
CGCTGTGCAGCCGACCGCCTGCCCACTATGGACAGCGGCCGCGGAAGCCA
TCACCATCACCATCACCATTAA

### Amino Acid Sequence (SEQ ID NO:6):

ADLGSSMEFEANAPGPVPGERQLAHSKMVPIPAGVFTMGTDDPQIKQDGE
APARRVTIDAFYMDAYEVSNTEFEKFVNSTGYLTEAEKFGDSFVFEGMLS
EQVKTNIQQAVAAAPWWLPVKGANWRHPEGPDSTILHRPDHPVLHVSWND
AVAYCTWAGKRLPTEAEWEYSCRGGLHNRLFPWGNKLQPKGQHYANIWQG
EFPVTNTGEDGFQGTAPVDAFPPNGYGLYNIVGNAWEWTSDWWTVHHSVE
ETLNPKGPPSGKDRVKKGGSYMCHRSYCYRYRCAARSQNTPDSSASNLGF
RCAADRLPTMDSGRGSHHHHHHH*

### Example 2

### Generation of Recombinant Baculovirus

The modified transfer vector pAcGP67 containing FGE cDNA and the BacMagic DNA-Kit from Novagen were used for generation of recombinant baculovirus according to the manufacturer's instructions. In detail, Sf9-cells (Invitrogen) were co-transfected with the transfer-vector and the provided BacMagic DNA using the following protocol:

1. 1×10⁶ cells in 2 ml BacVector® Insect Cell Medium were seeded in 35 mm plates at least 1 h before use. Plates were rocked gently in a side-to-side and back-and-forth pattern to ensure an even monolayer. The cells were allowed to attach to the surface of the plates for ˜1 h at 27° C.

2. A co-transfection mix of DNA and Insect GeneJuice® Transfection Reagent was prepared during the 1 h incubation period.

For each transfection, the following components were added to a sterile tube in the following order:

1 ml BacVector Insect Cell Medium

5 μl Insect GeneJuice

5 μl BacMagic DNA (100 ng total)

5 μl transfer vector DNA (500 ng total)

Total volume (1.015 ml)

Negative control:Instead of transfer vector DNA, corresponding amount of BacVector® Insect cell medium was added.

3. The components were mixed with vortexing for 10 sec.

4. It was then incubated at room temperature for 20-25 min to allow complexes to form.

5. Just prior to the end of the transfection mixture incubation period, culture medium from 35-mm plate(s) was removed carefully by using sterile pipette. Care was taken to not disturb the cell monolayer. When removing liquid from a dish of cells, the dish was tilted at a 30-60° angle, so the liquid pools to one side of the dish. The drying out of the cell monolayer was avoided.

6. Immediately after medium has been removed from cells, 1 ml of transfection mixture was added drop-wise to the center of dish. It was then incubated in a humidified container at 27° C. for 6 h.
7. After 6 h, 1 ml BacVector® Insect Cell Medium was added to each plate. Incubation was continued for 5 days in total. 8. After 5 days incubation, medium containing recombinant baculovirus were harvested (recombinant virus stock, 1st generation). Cells in the negative control formed a confluent monolayer. Virus-infected cells appeared grainy with enlarged nuclei.

### Example 3

### Amplification of Recombinant Virus Using Sf9 Cells Grown in Suspension Culture

1. 100 ml culture of Sf9 cells in a 500 mL shake flask at an appropriate cell density (e.g. 2×10⁶ Sf9 cells/ml in log phase growth, viability over 90%) was prepared. The cells were maintained at 27° C. in a shaker incubator at 185 rpm.

2. 0.5 ml of recombinant virus stock (1^{st} generation) was added to cell culture and cells were infected for 5 days. Under a phase-contrast inverted microscope the cells were checked for virus infection. The infected cells become grainy, uniformly rounded and enlarged, with distinct enlarged nuclei.

3. When cells appear to be well infected with virus, cell culture medium were harvested by centrifugation at 1000×g for 20 min at 4° C. Supernatants were removed aseptically by using sterilized pipette. Supernatant (recombinant virus stock, 2^{nd} generation) were stored at −20° C. or at −80° C. Multiple freeze/thaw cycles were avoided to maintain the virus titer.

### Example 4

### Expression of fl-FGE (WT)

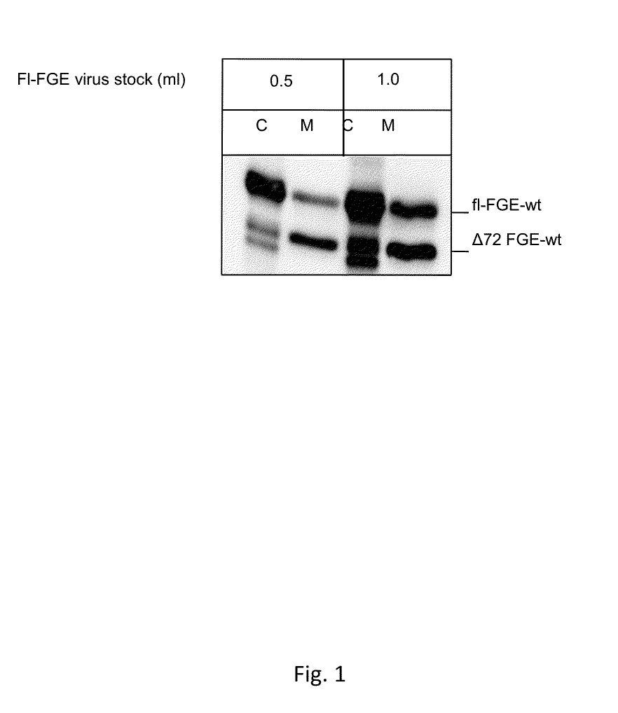

To check for the expression of fl-FGE (WT), 1×10⁶ High Five cells (Invitrogen) per mL were infected with either 0.5 ml or 1 ml of virus stock (second generation) in 50 ml Express five medium and cultured for 5 days at 27° C., 110 rpm. From these cultures, 100 μl aliquots were centrifuged at 100×g for 5 min and the supernatant (medium) was transferred to a new tube. The cell pellet was resuspended by boiling in 100 μl of 1× Laemmli buffer. Equal volumes of cells and medium were resolved in SDS-PAGE and the expression of fl-FGE (WT) in cells and medium was checked by western blotting using polyclonal FGE antiserum (FIG. 1).

A good expression of fl-FGE-wt was observable under tested conditions as shown in FIG. 1. However, note that a large fraction of secreted FGE is in the N-terminally truncated form (Δ72-FGE) due to furin-mediated processing, as has been shown earlier (Ennemann et al., 2013).

### Example 5

### Optimized Expression and Purification of fl-FGE-R69A/R72A

The expression conditions were optimized for the virus stock fl-FGE-R69A/R72A (SEQ ID NO: 4). Therefore 15 mL volumes of three times 0.5×10⁶ and four times 1×10⁶ High Five cells/mL suspensions were prepared in 50 mL bioreactor shakers. 150 μL, 300 μL and 1 mL of the virus stock were added to each cell density and the fourth 1×10⁶ cell/mL flask served as negative control. Suspensions were cultured for 5 days at 27° C., 185 rpm. Samples were taken every day, cells were spun down at 250×g and supernatants were stored at −20° C. 100 μL of the supernatants was separated by SDS-PAGE and were analyzed by western blotting using polyclonal FGE-antiserum.

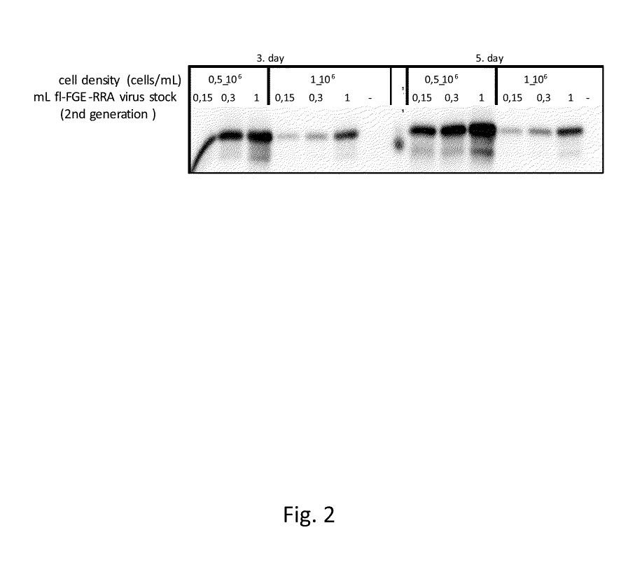

The western blot analysis (FIG. 2) showed that under these conditions a starting viable HighFive cell density of about 0.5×10⁶ is preferable. In addition, an increased volume of virus stock added led to an increased titer of FGE in the conditioned medium.

fl-FGE-R69A/R72A was produced by adding 13 mL of virus stock (second generation) to about 220 mL of 0.5×10⁶ cells/mL. The cells were cultured for 5 days at 27° C., 150 rpm in a sterile 1 L Erlenmeyer glass flask. The cells were spun down at 1000×g for 10 min at 4° C. and the culture supernatant (about 230 mL volume) was used for FGE purification on the same day.

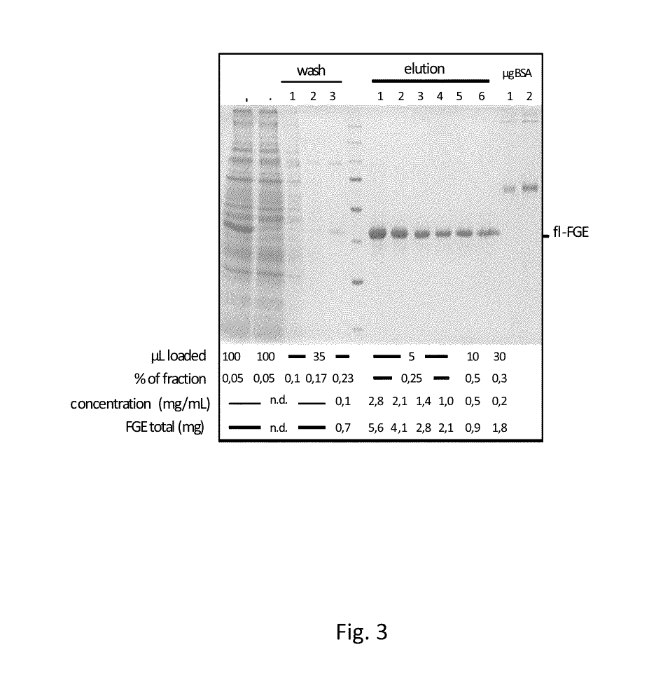

For purification a Ni-NTA agarose matrix from Qiagen was used for affinity purification via the Hiss-tag of the produced FGE. For the purification shown in FIG. 3 the beads (5 mL slurry) were first equilibrated in binding buffer (20 mM Tris, 100 mM NaCl, pH 8) before they were added to the fl-FGE-R69A/R72A (SEQ ID NO: 4) containing culture supernatant. Incubation was performed for 1 h, at 4° C. on a flask rotator. Afterwards, the matrix was spun down at 1000×g, 4° C. in a swing-out rotor for 5 min. The supernatant was collected (′ flow through′) and the affinity matrix pellet was resuspended in 35 mL of binding buffer. The sample was centrifuged as before. The supernatant was removed (Wash 1) and the affinity matrix was washed again in the same manner, once with wash buffer 2 (20 mL of 5 mM imidazole in binding buffer) and once with wash buffer 3 (10 mL of 15 mM imidazole in binding buffer). For elution, the matrix was resuspended in 2 mL of elution buffer 1 (250 mM imidazole in binding buffer). After one minute of incubation the matrix was spun down and the supernatant was collected as ‘elution fraction 1’. Elution was repeated in the same manner two times with 2 mL elution buffer 1, two times with 2 mL elution buffer 2 (500 mM imidazole in binding buffer) and finally one time with 10 mL of elution buffer 2. Aliquots of each fraction were boiled in Laemmli buffer for 5 min at 95° C. and were separated by SDS-PAGE for coomassie-staining analysis as shown in FIG. 3.

The coomassie-stained SDS-PAGE gel verifies the successful production and purification of FGE. A strong band for FGE was visible in the load fraction, which is missing in the flow through, demonstrating efficient binding of FGE to the Ni-NTA affinity matrix. Starting with the last washing step (Wash 3) the band corresponding to FGE appears at about 41 kDa size. The elution fractions clearly contained the purified fl-FGE-R69A/R72A(SEQ ID NO:4). In total, out of 230 mL culture volume about 17.2 mg FGE was purified. To summarize, the described culture conditions and purification method leads to a yield of about 75 mg fl-FGE-R69A/R72A (SEQ ID NO:4) per liter culture volume. Most important, FGE-R69A/R72A (SEQ ID NO:4) could be produced and purified as full-length enzyme. It is the first time that FGE could be purified without truncation.

### Example 6

### Optimized Expression and Purification of Δ72-FGE

The conditions for expression of truncated FGE (Δ72-FGE) (SEQ ID NO: 6) were optimized for Δ72-FGE virus stock (2^{nd} generation). High Five cells were grown in 100 ml Express Five Medium (Invitrogen), with two cell densities of 0.5×10⁶ cells/ml and 1.0×10⁶ cells/ml, in an Erlenmeyer flask. The cells were split into two 50 ml culture for each cell density. The cells were infected with either 0.5 mL or 1 mL of recombinant baculovirus for the aforementioned cell densities. The cells were infected until 96 hours at 27° C., 110 rpm. From these cultures, 100 μl aliquots were taken every 24 hours. The supernatant (medium) was transferred to a new tube after centrifugation at 100×g for 5 min and dried in a speed-vac. The pellet (cells) and the dried medium were resuspended in 100 μl of 1× Laemmli buffer and resolved by 10% SDS-PAGE. The expression of Δ72-FGE-wtin cells and medium was checked by either western blotting using polyclonal FGE antiserum or coomassie staining.

FIG. 5: Purification of Δ72-FGE by His-Trap affinity chromatography.

400 ml of conditioned express five media were subjected to His Trap affinity purification as described earlier in the text. The elution fractions after affinity purification were analyzed by SDS-PAGE and visualized by coomassie-staining. 50 μl of starting material (Load) and flow through (FT) and 20 μl of elution fractions (2%) were taken out from each fraction and were analyzed. Fractions containing Δ72-FGE that resolves at 37 kDa were pooled and the indicated protein concentrations were determined by Bradford assay.

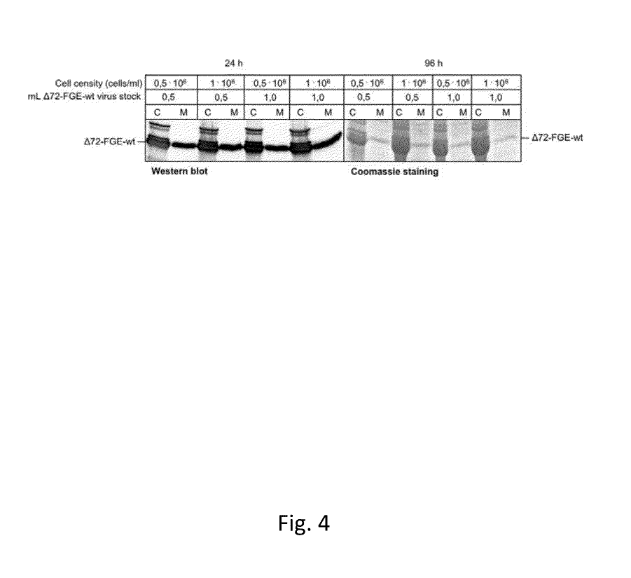

The results from western blot analysis and coomassie staining (FIG. 4) showed that the best expression under these conditions were achieved by a starting viable High Five cell density of about 1.0×10⁶ cells/mL (50 mL) infected with 1 ml of recombinant virus stock for 96 hours at 27° C.

High Five cells were grown to a density of 1×10⁶ cells/ml in 400 ml Express Five Medium. 8 ml of amplified 2^{nd} generation recombinant baculovirus was added to High five cells and the cells were infected for 96 h at 27° C. Cell supernatant was harvested by centrifugation at 3000 rpm at 4° C. The supernatant containing Δ72-FGE was later used for downstream processing.

The supernatant containing secreted Δ72-FGE was dialyzed against 5 L of buffer A (20 mMTris, 100 mM NaCl, pH 8.0) using 10 K MW cut-off dialysis tube (Snake Skin Dialysis tubing, 10K MWCO, 35 mm dry, Thermo Scientific) for 16 h at 4° C. After 8 h, old buffer A was replaced with 5 L of fresh buffer A and dialysis was continued for another 8 h at 4° C. The dialyzed material was filtered in 0.22-μm filter (Milipore) to remove any suspended particles. All the subsequent purification steps were performed at 4° C. using the Äkta purifier system. The filtered material was loaded onto a pre-equilibrated 1 ml His Trap HP column with a flow-rate of 0.5 ml/min using a 50 ml super-loop. The column was pre-equilibrated with buffer B (20 mMTris, 100 mM NaCl, 5 mM imidazole, pH 8.0) prior to loading. After loading, the column was washed with 10 column volumes (10 ml) of buffer B. Δ72-FGE-wt was eluted with a linear gradient of 0-100% buffer C (20 mMTris, 100 mMNaCl, 500 mM Imidazole pH 8.0) for 30 min at a flow rate of 1 ml/min; 1-ml elution fractions were collected. Aliquots of each fraction were boiled in 1× Laemmli buffer for 5 min at 95° C. and were separated by SDS-PAGE for coomassie-staining analysis as shown in FIG. 5.

The results from coomassie-stained SDS-PAGE gel demonstrate the successful production and purification of FGE. A prominent band appearing at ˜37 kDa in the elution fractions 3 to 7, represents the purified Δ72-FGE (shown in FIG. 4). The identity of protein was further determined by LC MALDI MS/MS and data base search (FIG. 6). In total, out of 400 mL culture volume about 6.75 mg FGE was purified. The total yield of purified Δ72-FGE was around 17 mg per liter culture volume.

### Example 7

### Comparison of the Expression Systems

With the mammalian cell line HT1080 stably producing FGE-His6, 2 mg FGE could be purified out of one liter conditioned medium (Preusser-Kunze et al. 2005). However, more than 90% of this FGE lacks the N-terminal sequence up to amino acid position 72. It has been shown that Δ72-FGE purified from mammalian cell culture supernatant has a specific activity between 60 mU/mg and 137 mU/mg under in vitro conditions (Preusser-Kunze et al. 2005; Peng et al. unpublished). By contrast, we describe here that 17 mg of purified Δ72-FGE (SEQ ID NO:6) could be obtained from the Baculo-virus expression system per liter conditioned Express Five Medium yielding a fully functional protein (specific activity 70-90 mU/mg) (Alam, Thesis 2013, unpublished).

Most notably, even 75 mg of purified fl-FGE-R69A/R72A (SEQ ID NO:4) could be obtained per liter conditioned medium with the Baculovirus expression system showing a specific activity of 33 mU/mg (Wachs et al., unpublished).

### Example 8

### In Vitro Activity of fl FGE with Gluthatione and DTT

a) fl-FGE-R69A/R72A (SEQ ID NO: 4) is active and e.g. generates FGly within a 23-aa long substrate peptide 16 pmol of the 23 amino acid (aa) long peptide Ac-MTDFYVPVSLCTPSRAALLTGRS-NH₂ (SEQ ID NO:46) were incubated with 12 ng of fl-FGE-R69A/R72A for 20 min at 37° C. under assay conditions (50 mM Tris, 67 mM NaCl, 15 μM CaCl₂, 0.33 mg/mL BSA, 2 mM DTT, pH 9.3 in a total volume of 30 μL).

The reaction was stopped by adding 3 μL of 20% trifluoroacetic-acid (TFA), immediately followed by vortexing and by a short centrifugation at 10000×g. The peptide was purified and concentrated by C18-Zip-Tip treatment. Therefore the Zip-Tip was prepared by pipetting three times 10 μL of 50% acetonitrile, 0.05% TFA in water and three times 10 μL 0.1% TFA in water. The 33 μL sample was pipetted 10 times up and down. The bound peptide was washed by pipetting 10 times 10 μL 0.1% TFA in water and was eluted in 10 μL 50% acetonitrile, 0.05% TFA in water by pipetting 10 times up and down. For MS-analysis the following matrix was prepared freshly: 40 μL of a saturated α-cyano-hydroxycinnamic acid solution in aceton were added to 10 μL of a solution containing 10 mg/mL nitrocellulose in 50% Aceton/50% Isopropanol (v/v). 0.5 μL of the matrix were spotted onto a polished steel target and 1 μL of the purified sample was added. The dried sample spot was analyzed by MALDI-ToF mass spectrometry using the Ultra flextreme spectrometer from Bruker Daltronics. The cysteine containing substrate peptide (2526.28 [M+H]⁺) and the FGly containing product peptide (2508.29 [M+H]⁺) were detected.

The Δ72-FGE (SEQ ID NO: 6) variant also converts the cysteine-containing substrate peptide to the FGly-containing product peptide in presence of the reductant DTT.

b) fl-FGE-R69A/R72A (SEQ ID NO:4) is able to generate FGly by using GSH as reductant:10 times stock solutions of DTT and GSH were prepared in 20 mM Tris pH 9.3. For the reaction, 13 ng of Baculo-fl-FGE were incubated with 16 pmol of the 23 aa-long substrate peptide, 2 mM DTT or 5 mM reduced glutathione for 15 min at 37° C. under assay conditions (50 mM Tris, 67 mM NaCl, 15 μM CaCl₂, 0.33 mg/mL BSA, pH 9.3 in a total volume of 30 μL). The reactions were stopped and prepared further for MS-analysis as described above. The ratio of the intensity of the FGly-product peptide divided by the sum of the substrate and product intensities multiplied with 100 yields %-turnover of the substrate peptide. Based on that, specific activities were calculated.

The tested aliquot of fl-FGE-R69A/R72A (SEQ ID NO:4) was active in presence of GSH. The specific activities determined (4 measurements each) were 15.6±1.3 mU/mg in presence of GSH and 24.1±1.7 mU/mg in presence of the control reductant DTT (M. Wachs, unpublished).

### Example 9

### FGE in Human Expression System

### Expression Plasmids

The construction of plasmids used to express FGE tagged C-terminally with either -RGS-His6 (pSB-FGE-His) or -HA (pBI-FGE-HA), FGE with an appended KDEL motif at the C-terminus (pBI-FGE-HA-KDEL), steroid sulfatase (pBI-STS) and myc-ERp44 (pBI-myc-ERp44) were described earlier (12, 15). All variants of the cleavage site motif of FGE used in this study were generated by site-directed mutagenesis PCR with pBI-FGE-HA as template and Long-template Expand PCR system (Roche). The coding sequences of the primers were:

(SEQ ID NO: 7 R69A)
TCGGCAGCCGCTCACGCATACTCGCGGGAGGCT,
(SEQ ID NO: 9 R69K)
TCGGCAGCCGCTCACAAATACTCGCGGGAGGCT
(SEQ ID NO: 11 Y70A)
GCAGCCGCTCACCGAGCCTCGCGGGAGGCTAAC
(SEQ ID NO: 13 Y70K)
GCAGCCGCTCACCGAAAGTCGCGGGAGGCTAAC
(SEQ ID NO: 15 Y70F)
GCAGCCGCTCACCGATTCTCGCGGGAGGCTAAC
(SEQ ID NO: 17 Y70S)
GCAGCCGCTCACCGATCCTCGCGGGAGGCTAAC,
(SEQ ID NO: 19 S71A)
GCCGCTCACCGATACGCGCGGGAGGCTAACGCT
(SEQ ID NO: 21 S71R)
GCCGCTCACCGATACAGGCGGGAGGCTAACGCT
(SEQ ID NO: 23 R72K)
GCTCACCGATACTCGAAGGAGGCTAACGCTCCG
(SEQ ID NO: 25 R72A)
GCTCACCGATACTCGGCGGAGGCTAACGCTCCG
(SEQ ID NO: 27 R69A/R72A)
GCTCACGCATACTCGGCGGAGGCTAACGCTCCG
(SEQ ID NO: 28 Y70AS71R)
GCCGCTCACCGAGCCAGGCGGGAGGCTAACGCT
(SEQ ID NO: 30 E73P)
CACCGATACTCGCGGCCGGCTAACGCTCCGGGC.

The resulting constructs were verified by full-length sequencing of the coding region to exclude any PCR prone errors. Plasmids for expression of the PCs encoding human furin (Gene ID 5045), human PACE4 (=PCSK6, Gene ID 5046), murine PCSa (=Pcsk5, Gene ID 18552) and rat PC7 (=Pcsk7, Gene ID 29606) were kindly provided by Abdel-Majid Khatib (18). Note that the PC cDNAs were encoded as pIRES constructs coding also for enhanced green fluorescence protein (EGFP) such that EGFP expression levels serve as a measure of the expression levels of PCs.

### Generation of the Phylogenetic Tree and Sequence Logos

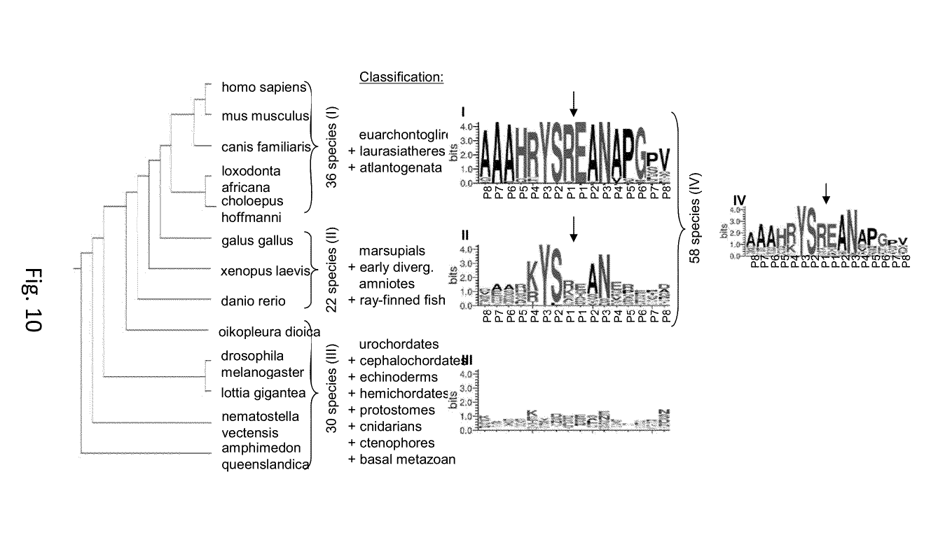

The phylogenetic tree (FIG. 10, left) is based on the Newick format of 13 representative species out of a total of 88 SUMF1 sequences from different species. For sequence logo generation through the WebLogo 3.0 program (19, 20) species could be divided into three subgroups, based on the presence of the motif R-Y-S-R (group I) or R/K/X-Y-S-R/K/X (SEQ ID NO:44) (group II; X denotes any amino acid) or no common sequence (group III) at position P1-P4 of the cleavage site in most species of the given classifications (FIG. 10). The sequences were centered at P1 (Arg72) or based on a ClustalW alignment in case of the sequences of group III that do not comprise a R/K/X-Y-S-R/K/X (SEQ ID NO:44) motif 16 amino acids corresponding to positions 65-80 in human FGE are displayed. All 88 sequences are listed as supplementary data (Table 2).

### Cell Culture and Transfection

HT1080, HeLa, HEK 293 and BHK cells (American Type Culture Collection, USA) were cultured at 37° C. under 5% CO₂ in Dulbecco's modified Eagle's medium (Invitrogen) containing 10% fetal calf serum (FCS) (Lonza). CHO-K1 and furin-deficient CHO-FD11 cells (kindly provided by Steven Leppla (21)) were cultured in DMEM supplemented with 40 μg/mL proline (Fisher Scientific).

### Generation of Furin Deficient CHO-FD11 Cells

Furin deficient CHO cells (CHO-FD11) as mentioned above, are well known (Susan-Resiga, et al., 2011 The Journal of Biological Chemistry, 286, 22785-22794. Zhang et al., J Virol. Mar. 2003; 77(5): 2981-2989, Nour et al., Mol. Biol. Cell Nov. 1, 2005 vol. 16 no. 11 5215-5226 or Pilz et al., Virology, vol. 428, Issue 1, 20 Jun. 2012, Pages 58-63) and further described in detail in Gordon et al, 1995, in Infect. Immun, 63 (1) (1995), pp. 82-87. In particular, Furin deficient CHO cells (CHO-FD11) are were generated from CHO-K1 cells that are resistant to bacterial toxins. Several toxins like Diptheria toxin (DT), protective antigen (PA) from Bacillus anthracis and Pseudomonas exotoxin A (PE) induce cytotoxicity only upon activation by proteolytic cleavage mediated by cellular proteases. By treatment of CHO-K1 cells with recombinant bacterial toxins, one can generate mutant CHO cells that are resistant to bacterial toxin-mediated cytotoxicity, thereby deficient in proteases responsible for activation of the bacterial toxins. The method for the generation of CHO-FD11 cells that are deficient for the protease furin is described in Gordon et al., (21). Briefly, CHO-K1 cells (obtained from American Type Culture Collection ATCC CCL-61) are grown in T-75 flasks to 80% confluency. Cells are treated with 6 μl ethyl methanesulfonate (EMS) per 20 ml of medium for 18 h at 37° C. After washing with fresh medium, 5×10⁵ cells per ml are plated in 100 mm diameter dishes and incubated for further 5 days at 37° C. The cells are then treated with recombinant bacterial toxins viz., 50 ng of FP50 (a derivative of PE) per ml in combination with 100 to 100 ng/ml PA toxin derivatives. 36 h post-treatment, the medium containing the toxin is removed and replenished with fresh medium. Surviving colonies are screened for their sensitivity towards bacterial toxins PE, DT, PA and cleavage site mutants of PA and resistant clones were generated by limiting dilution.

HT1080 Tet-On and MSDiTet-On cells, for doxycycline inducible protein expression were maintained as described earlier (12). All transfections were performed with lipofectamine LTX as recommended by the manufacturer (Invitrogen). A stable CHO-FD11 Tet-On cell line was generated by transfection of CHO-FD11 cells with pUHrT62 (kindly provided by Nadja Jung) encoding the reverse tetracycline controlled transactivator and neomycin resistance vector pSB4.7 pA in a 10:1 ratio (15). The stable clones were selected with medium containing 0.8 mg/mL neomycin (Invitrogen) and screened through western blotting for doxycycline-dependent FGE expression after transient transfection with pBI-FGE-HA plasmid. CHO-FD11 cells stably expressing FGE-RGS-His6 were generated by transfecting CHO-FD11 cells with pSB-FGE-RGS-His6 and transfectants were selected with 0.8 mg/mL G-418 sulfate (PAA). The stable clone was selected through FGE expression analysis by western blotting.

### Preparation of Cell and Medium Samples for SDS-PAGE and Western Blot Analysis

3 μg of pSB-FGE-RGS-His6 vector was used for single transfections of HT1080, HeLa, HEK 293, BHK and CHO cells. Conditioned medium was collected after 24 h and centrifuged (500×g, 5 min) Cells were washed once with PBS, treated with trypsin (Lonza) and pelleted by 250×g to remove the medium. Cell pellets were resuspended in PBS (pH 7.4) containing protease inhibitor (PI) cocktail (Sigma) and lysed by sonication 3×10 s on ice. For co-expression of FGE with one of the PCs in CHO-FD11 Tet-On cells 2 μg of pBI-FGE-HA and 2 μg of the plasmids encoding PCs (see above) were used for transient transfection. In case of transient transfections using pBI-vector constructs the protein expression was induced by replenishing the medium with medium containing 2 μg/mL doxycycline (BD Biosciences) at 5 h post-transfection. After induction for 24 h, the cells and media were collected and processed as described above for further analysis by SDS-PAGE and western blotting.

Western blot analyses were carried out using rabbit polyclonal antiserum against FGE and rabbit polyclonal anti-GFP antibody (Living Colors® A.v. Peptide Antibody, Clontech) as primary antibodies and the peroxidase-conjugated goat anti-rabbit secondary antibody (Invitrogen). Quantification of western blot signals was performed using AIDA 2.1 software package (Raytest) and calculation of FGE amounts were based on 10 or 20 ng FGE standard signals, present on the same blot.

### Immunoprecipitation

HT1080 cells were cultured in four 14 cm plates with 5% FCS containing medium for 48 h. Medium was collected and centrifuged 10 min at 1000×g, to remove cell debris. Cells of one plate were harvested by trypsinisation, pelleted at 1000×g for 5 min and lysed by sonication in PBS (pH 7.4) containing 0.1% Triton X100 and PI cocktail, followed by centrifugation at 20 000×g for 15 min. As a negative control fresh medium containing 5% FCS was used and 0.01% Triton X100 and PI cocktail was added to it and the cleared medium. All supernatants were pre-incubated with rabbit preimmune serum for 30 min at 4° C. and after addition of ProteinA-Sepharose CL-4B (Sigma-Aldrich) the bound material was pelleted down by centrifugation at 7000×g for 10 min. The supernatants of the conditioned medium and the cell lysate were split into two parts and either rabbit preimmune serum or rabbit FGE antiserum was added. After incubation at 4° C. and addition of ProteinA-Sepharose CL-4B the bound material was pelleted down by centrifugation at 7000×g for 10 min. The pellets were washed stepwise as described earlier (22). The pellets were boiled in 1× Laemmli buffer for 5 min at 95° C. and centrifuged at maximum speed for 5 min. 100% of the medium and 30% of the cell lysate supernatants were loaded for 12.5% SDS-PAGE followed by western blotting and detection with FGE antiserum.

### Steroid Sulfatase (STS) Activity Assay and Western Blotting

Activity assays of STS were performed as described earlier (15, 23). For western blot analysis, polyclonal antisera against FGE and steroid sulfatase were used as primary antibodies. Horseradish peroxidase-conjugated goat anti-rabbit antibody was used as secondary antibodies. Signals of steroid sulfatase are given as relative amounts, i.e. related to signal intensities detected in cells expressing steroid sulfatase only. Relative specific sulfatase activities were calculated, i.e. catalytic activity divided by the western blot signal (arbitrary units) and referred to that of cells expressing the sulfatase only (relative specific sulfatase activity=1). Absolute values for this reference are given in the legends.

In Vitro Furin Cleavage Assay and Treatment of Cells with RVKR-CMK Inhibitor

CHO-FD11 cells stably expressing FGE-His6 or HT1080 cells were grown for 24 h, and cells and medium were harvested. For treatment with furin in vitro cell pellets were resuspended in HEPES pH 7.5 buffer containing 1 mM CaCl₂ and 0.5% Triton X-100 and lysed in the absence of protease inhibitors by sonication on ice. Appropriate amounts of cell lysate and medium were incubated for 3 h at 25° C. with 4 units of furin (NEB) with or without 25 μM decanoyl-RVKR-CMK (Alexis biochemicals) as indicated in the figures. For in vivo treatment, HT1080 Tet-On cells were transiently transfected with pBI-FGE-HA and 4 h post-transfection, the medium was replenished with medium containing 20 ng/mL doxycycline and various concentrations of decanoyl-RVKR-CMK or only DMSO as a carrier control. The cells and medium were collected after 16 h of induction/treatment for further analysis. The samples were boiled in Laemmli buffer and analyzed by SDS-PAGE and western blotting with FGE antiserum.

### Extracellular Processing of FGE Containing Conditioned Medium

CHO-FD cells stably expressing FGE-His6 were cultured on 25 cm plates in 15 mL 5% FCS containing medium for 24 h. Medium was centrifuged at 500×g and supernatants were sterile filtered to remove any floating producer cells. The conditioned medium was added to confluent 10 cm plates of CHO-FD11 (negative control), MSDi, HT1080, HeLa and HEK 293 cells for the indicated incubation times. Medium was collected and centrifuged at 500×g. Equal amounts of medium were used for western blot analysis.

### In Vitro FGE Activity Assay

FGE activity was assayed using conditioned media that contained either secreted fl-FGE (obtained from CHO-FD FGE-His₆ cells) or Δ72-FGE (obtained from CHO-FD Tet-On cells after transient co-transfection with pBI-FGE-HA and pIRES-Furin constructs). After expression for 48 h at 1% or 2.5% FCS containing DMEM, the medium was collected and centrifuged at 500×g for 5 min. The supernatant was analyzed by SDS-PAGE and western blotting and aliquots were directly used for activity testing.

The activity assay was performed in triplicates with three sets of conditioned media at pH 9.3 under standard conditions as described earlier (4) with some modifications; no BSA was added to the reaction mixture and 2 mM DTT or 5 mM GSH were used as reducing agents.

### Example 10

### FGE is N-Terminally Truncated During Secretion by a Non-Saturable Mechanism in a Post-ER Compartment

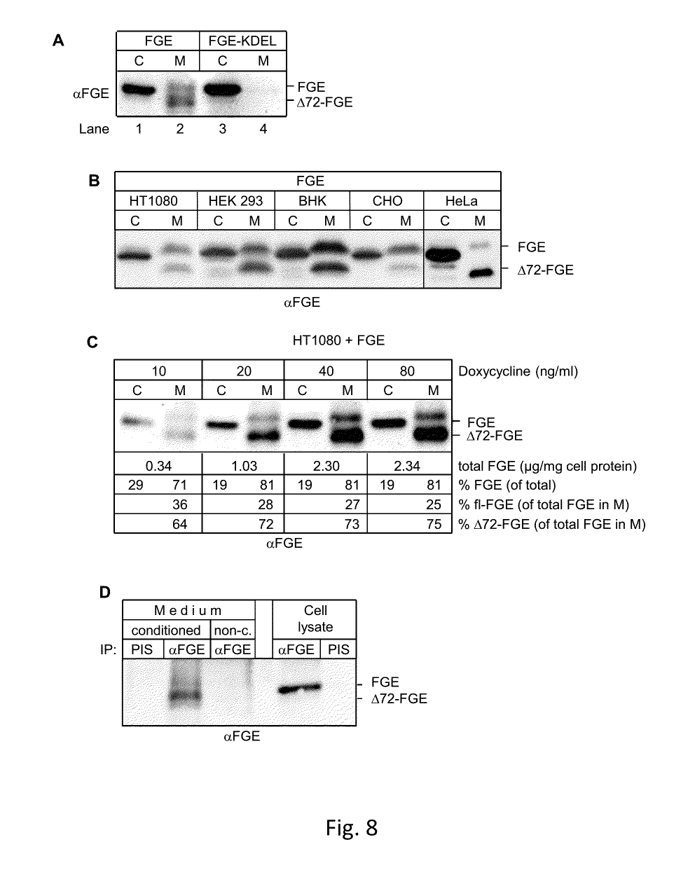

FGE, an ER localized enzyme, lacks the canonical (KDEL-like) ER-retention signal and is retained in the ER via interactions with ERp44 by a saturable mechanism (13). It is known that recombinantly expressed FGE (41 kDa) in HT1080 cells is released into the medium and the majority of the secreted protein represents a N-terminally truncated form of 37 kDa in size lacking residues 34-72 (Δ72-FGE) (15). To examine whether this proteolytic processing occurs in a compartment along the secretory route, the inventors expressed FGE and FGE with an appended KDEL sequence at the C-terminus (FGE+KDEL) in HT1080 cells (FIG. 8A). Analysis of the cell homogenate and medium clearly shows that the FGE population escaping the ER retention machinery is eventually secreted (FIG. 8A, lane 2) and thereby proteolytically processed, whereas retaining FGE in the ER through an appended KDEL sequence prevents the secretion and in turn the proteolytic processing (FIG. 8A, lane 3 and 4). These data indicate that the transport of FGE to a post-cis-Golgi compartment during secretion is a prerequisite for cleavage. It should be noted that the unprocessed FGE in the medium (see also FIG. 8C) has a lower electrophoretic mobility in SDS-PAGE compared to the intracellular FGE due to modification of the attached N-glycan in the secretory pathway (see ref 15).

To analyze the proteolytic processing in detail and to exclude any cell type-specific effect, the inventors studied the secretion profile of FGE in various cell lines. FGE was transiently expressed in HT1080, HeLa, HEK293, BHK and CHO cells. The analysis of the cell homogenate and medium revealed that FGE is secreted in all cell lines tested (FIG. 8B) and that usually the majority of the secreted FGE is in the truncated form. Of note, the extent of processing was comparable across cell lines except in CHO cells, wherein the truncation was less pronounced. The secretion of the unprocessed full-length (fl) form suggests that the processing mechanism could be saturated due to high FGE expression levels. To test this hypothesis, FGE was expressed under the control of a doxycycline inducible promoter in HT1080 Tet-On cells. FGE expression was induced with increasing concentrations of doxycycline and quantification of the amount of fl- and Δ72-FGE in the secretions shows that even a seven-fold increased FGE expression does not increase the proportion of the fl form in the medium (FIG. 8C). About 25-35% of secreted FGE is in the unprocessed form independent of the expression level indicating that incomplete processing is not due to saturation of the processing machinery. To determine whether also endogenous FGE is secreted and whether it is secreted in a processed form, Subsequently FGE from untransfected HT1080 and HeLa cell homogenate and medium samples were immunoprecipitated. In both cell lines FGE was traceable in the secretions, as shown in FIG. 8D for HT1080 cells. The majority of secreted FGE was in the truncated form and surprisingly the unprocessed form was also detected to similar relative levels as observed under overexpression conditions.

### Example 11

### The RYSR Motif in the N-Terminus of FGE is Indispensable for Proteolytic Processing of Secreted FGE but not Required for FGE Activity In Vivo

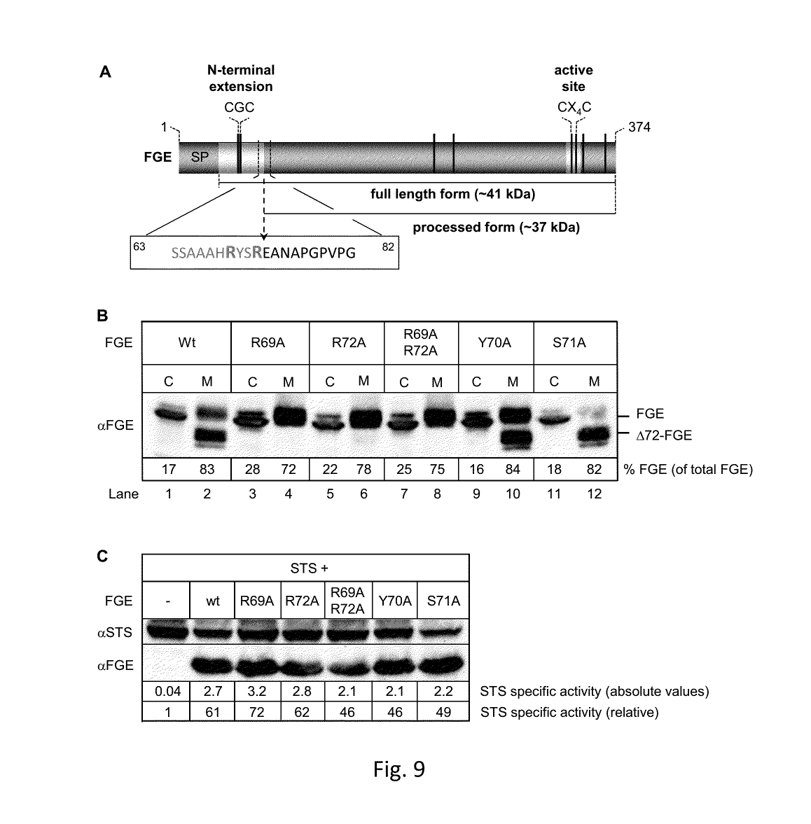

Since many secreted glycoproteins that are processed along the conventional secretory pathway contain a R-X-X-R-like motif, the inventors speculated that the RYSR motif (SEQ ID NO: 48) in FGE (residues 69-72, FIG. 9A) represents a potential proproteinconvertase (PC) cleavage site. This is supported by our previous finding that the N-terminally truncated FGE starts at glutamate 73 (15). Surprisingly, an in silico analysis using a widely used PC cleavage site prediction program (24) did not yield any potential cleavage motif in FGE and only a recently published program “PiTou” predicts a cleavage at the RYSR motif (SEQ ID NO: 48), albeit with a low score (+0.93) (25).

To analyze biochemically the role of the R-X-X-R-like motif in the N-terminus of FGE, the following alanine variants were generated: FGE-R69A (SEQ ID NO: 8), -R72A (SEQ ID NO: 26), -R69A/R72A (SEQ ID NO: 4), -Y70A (SEQ ID NO: 12) and S71A (SEQ ID NO: 20). Expression of FGE-R69A, -R72A or -R69A/R72A led to a clear resistance to truncation and all secreted FGE was in the unprocessed fl-form (FIG. 9B, lanes 4, 6, 8) indicating that both arginine residues (Arg69 and Arg72) are equally essential for proteolytic cleavage. Expression of FGE-S71A led to a nearly complete processing of secreted FGE (FIG. 2B, lane 12), whereas the Y70A mutation conferred partial resistance to cleavage, with 1.5 fold more FGE secreted in the fl-form compared to wildtype (wt) FGE (FIG. 9B, compare lanes 2 and 10). These data indicate that the RYSR motif is a bona fide cleavage motif and that residues Tyr70 and Ser71 determine cleavage efficiency.

Expression of sulfatases in immortalized MSD cells which lack endogenous FGE activity leads to the production of inactive sulfatases whereas co-expression of a sulfatase with FGE in these cells yields active sulfatases, thus providing a reliable system to investigate FGE mediated FGly generation in vivo (12). Using this method the inventors examined whether the RYSR motif is required for the activation of sulfatases. cDNAs encoding steroid sulfatase (STS) alone or co-transfected along with cDNAs encoding RYSR-motif alanine variants of FGE in immortalized MSD Tet-On cells were transfected (FIG. 9C). Upon doxycycline induction, STS was barely active when expressed alone, whereas co-expression with FGE-wt increased the activity of STS approx. 50-fold, as observed previously (12). Co-expressing the alanine variants of the RYSR motif led to an increase in STS activity to similar levels as observed for FGE-wt, indicating that the integrity of the RYSR motif is not required for the activity of FGE in vivo. In conclusion, the RYSR motif in the N-terminus of FGE serves as an authentic cleavage motif rather than a role in the activity of cellular FGE.

### Example 12

### The RYSR Motif is Highly Conserved Among Higher Eukaryotes

Since the RYSR motif was only recognized as a weak potential cleavage site by the prediction program, the inventors analyzed the conservation of the cleavage site flanking regions across species in the animal kingdom. The whole N-terminus including the cleavage site as well as the important Cys-Gly-Cys-motif that has been studied earlier (12, 13) is encoded by exon1 of the SUMF1 gene. Notably, this region has no counterpart in homologous prokaryotic genes (26) and in fact arose as an extension in early eukaryotes and persisted to their present day descendants.

A phylogenetic tree with 13 representative species (FIG. 10, left) provides an overview for our set of 88 available eukaryotic sequences, which were arranged according to modern molecular taxonomy (27, see Table 2 for the full set of sequences). Within this taxonomy-based list of species the inventors could identify three groups. Group III consisted of the 30 earlier diverging eukaryote species from sponge (basal metazoan) to sea pineapple (urochordates), among which no significant sequence conservation was detected in the relevant region (FIG. 10, WebLogo III). By contrast, in the 58 later diverging eukaryote species from northern pike (ray-finned fish) to human the sequences are significantly conserved forming the cleavage site motif K/R-YS-R/K (SEQ ID NO: 50) (FIG. 10, WebLogo IV). The 22 species from northern pike to short tailed opossum (marsupials) mainly carry either a Lys- or an Arg-residue at positions P4 and P1 (group II), while the 36 sequences representing the full range of placental mammal species form a highly conserved RYSR core motif (SEQ ID NO: 48) (group I). Of note, some of the earlier diverging eukaryote species (group III) do also bear a similar motif like the later diverging eukaryotes, e.g. KYKR (SEQ ID NO: 51) in the mountain pine beetle (dendroctonusponderosae) (Table 2).

Interestingly, especially the residues Tyr70 and Ser71 within this motif are almost 100% conserved among the 58 later diverging eukaryotes and, additionally, Glu73, Ala74 and Asn75, representing the neo-N-terminus of processed FGE, are as highly conserved as the R/K-YS-R/K (SEQ ID NO: 50) itself (FIG. 10, WebLogo IV). The high degree of conservation of these seven consecutive residues in later diverging eukaryotes is best explainable by a strong selective pressure implying important functionality of this sequence.

Notably, all 88 species from human to sponge contain the essential Cys-Gly-Cys sequence mentioned above, which is required for sulfatase activation (12). Therefore the cleavage site motif arose later in the molecular evolution of the N-terminus, and gained a function that may serve as a tool to regulate FGE activity in later diverging eukaryotes.

### Example 13

### RYSR⁷²↓ E Represents a Unique Cleavage Motif that Imparts Suboptimal Cleavage Efficiency

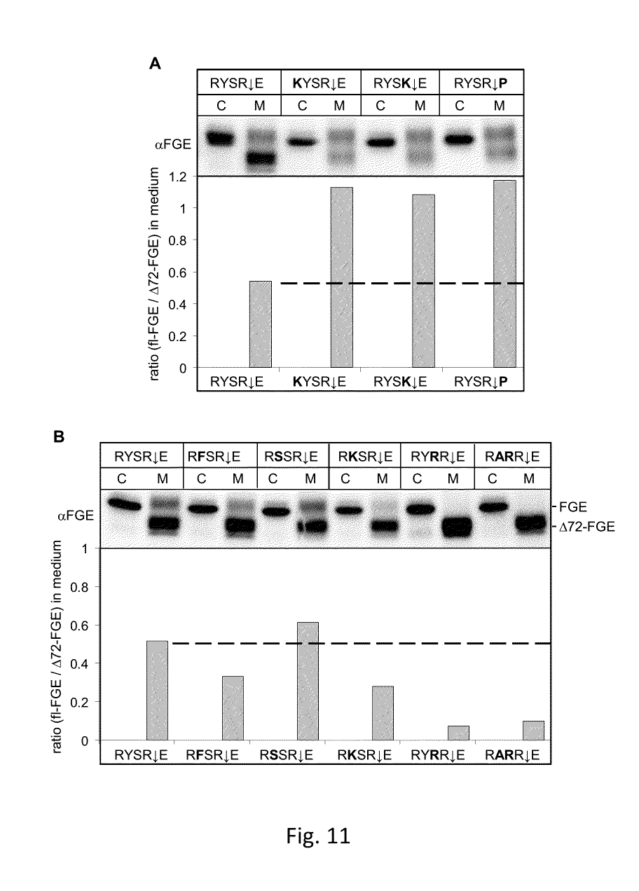

Our observation that under any condition tested approx. 20-30% of secreted FGE-wt is in the unprocessed form (FIGS. 8 and 9) suggested that the processing of the N-terminal extension of secreted FGE is suboptimal. The cleavage efficiency of mammalian PCs has been shown to be directly dependent on ˜20 amino acid residues surrounding the cleavage site and especially the positions P4 to P1′ (RYSR⁷²↓ E in FGE) are important. To determine biochemically the functional advantage of the conserved residues in the RYSR⁷²↓ E motif, the inventors transiently expressed motif variants in HT1080 cells and quantified the extent of processing in the secretions by SDS-PAGE and western blotting. Expression of the motif variants KYSR⁷²↓ E (SEQ ID NO: 10) and RYSK⁷²↓ E, (SEQ ID NO: 24) representing motifs found in group II species that contain Lys at positions P4 or P1 respectively, led to truncation of the secreted protein, but the cleavage efficiency was lowered, as indicated by a doubling of the fl-FGE/Δ72-FGE (SEQ ID NO: 6) ratio compared to that of FGE-wt (SEQ ID NO: 2) (FIG. 11A). The occurrence of proline at position P1′ is known to compromise the cleavage efficiency of the PCs and as expected, substitution of Glu73 with proline (RYSR⁷²↓ P) (SEQ ID NO: 30) also led to a reduction in processing indicating that Glu73 is conducive to cleavage.

Mutating the highly conserved Tyr70 at position P3 to Phe (RFSR↓E) (SEQ ID NO: 16) or Lys (RKSR↓E) (SEQ ID NO: 14) led to a clear increase in cleavage efficiency, while substitution with Ser (RSSR↓E) only had a minor effect (FIG. 11B). A dramatic increase in the cleavage efficiency was observed when Ser71 at position P2 was mutated to a positively charged arginine residue (RYRR↓E or RARR↓E) (SEQ ID NO: 22 and 28) to a level where virtually all of the secreted FGE was processed. From these data the inventors conclude that RYSR↓E is a unique cleavage motif with the highly conserved Tyr (at position P3) and Ser (P2) conferring an inefficient cleavage property to FGE. Whether these residues, apart from probably being unfavourable for PC recognition, make contact to the FGE core domain (or to other proteins), which may restrict access of the cleavage site, remains to be determined

### Example 14

### Furin Mediates the Proteolytic Processing of Secreted FGE

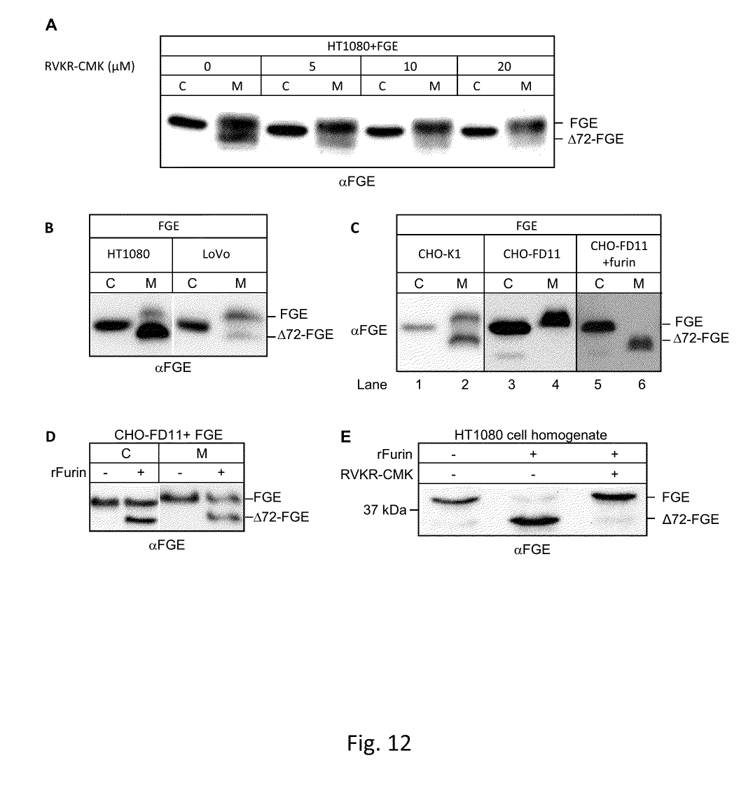

In order to verify that the N-terminus of FGE containing the RYSR motif is subject to processing by furin or furin-like convertases, the inventors studied the secretion profile of FGE in cells in the presence of decanoyl-RVKR-ChloroMethylKetone (CMK), an inhibitor for PCs (FIG. 12A). HT1080 Tet-On cells expressing FGE under the control of doxycycline were treated with only DMSO (as a carrier control) or increasing concentrations of the inhibitor for 16 h in culture. Indeed the amount of the truncated form of FGE in the secretions was decreased by the inhibitor in a concentration dependent manner (FIG. 12A). These data clearly indicate that the N-terminal processing during secretion of FGE is mediated by PCs.

In mammals, the proproteinconvertase family comprises nine enzymes that differ in their substrate specificity and tissue-specific expression and/or subcellular localization (17). Furin is the best characterized mammalian PC with a ubiquitous tissue distribution and it is the major convertase in the secretory pathway. To analyze the role of furin in the processing of FGE the inventors expressed FGE in cells that are deficient for furin. When expressed in LoVo cells, the major fraction of secreted FGE in the medium was found in the unprocessed form, which contrasts with the observations in other cells; however, a significant fraction was still in the truncated form (FIG. 5B). Strikingly, expression of FGE in furin-deficient CHO cells (CHO-FD11) led to secretion of FGE in the full-length form exclusively (FIG. 12C, lane 4), while expressing FGE in CHO-K1 cells, which contain endogenous furin, led to processed FGE in the secretion medium (FIG. 12C, lane 2). This clearly indicates that furin is involved in processing the N-terminus of FGE. Remarkably, when furin was replenished in CHO-FD11 cells by exogenous expression, all secreted FGE was in the truncated form (FIG. 12C, lane 6) providing unequivocal evidence that FGE is processed by furin along the secretory pathway. Further FGE was found to be processed by furin in vitro. When cell homogenate and medium from CHO-FD11 cells stably expressing FGE-His were treated with commercially available recombinant furin (rFurin), almost half the amount of FGE was processed, as observed for both cells and medium (FIG. 12D). Using this in vitro furin cleavage assay, the inventors also examined whether endogenous intracellular FGE is susceptible to furin processing. Indeed, rFurin readily converted almost 90% of endogenous fl-FGE from untransfected HT1080 cells to the truncated form (FIG. 12E). Also here the RVKR-CMK inhibitor completely abrogated the furin-mediated processing of FGE. In summary, these data unambiguously show that furin mediates the N-terminal truncation of FGE.

### Example 15

### Other Members of the PC Family are Able to Process FGE During Secretion and in Some Cells Processing Additionally Occurs Extracellularly

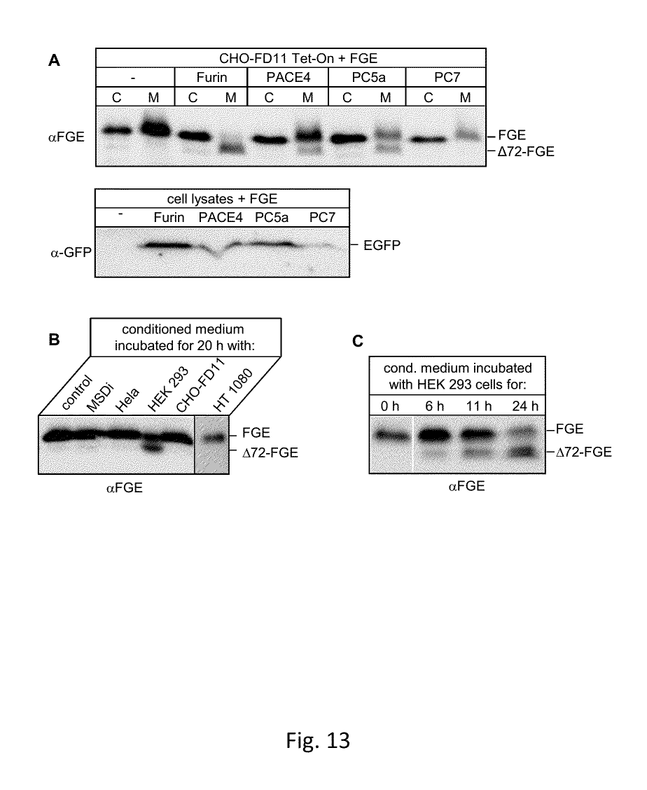

The observation of a low but significant truncation of secreted FGE in LoVo cells (FIG. 12B) led us to speculate that FGE could also serve as a substrate to other furin related PCs. It is reported that PACE4, PCSa and PC7 have sequence specificity similar to that of furin (28, 29). In order to verify this, pIRES vector constructs coding for EGFP and furin, PACE4, PCSa or PC7 were transiently transfected along with pBI-FGE in CHO-FD11 Tet-On cells; thereafter cell and medium samples were subjected to western blot analysis for FGE processing (FIG. 13A). Successful detection of EGFP signals in transfected cell lysates indirectly verified expression of un-tagged PCs (FIG. 13A, lower panel). Coexpression of furin led to nearly 100% Δ72-FGE in secretions, whereas PACE4 and PC5a coexpression led to processing of FGE, albeit with low efficiency. Of note, a physiological role for PC7 on the processing of FGE is unclear due to its lower expression level compared to other PCs. However, this experiment indicates that FGE is a better substrate for furin than for other PCs.

PCs are mainly localized in the trans-Golgi network (TGN) but do also cycle between the endosomal compartments and the cell surface (30). In addition, a soluble form of furin can be generated by sheddases (31). To investigate the possibility of post-secretion processing, the inventors incubated fl-FGE containing conditioned medium obtained from CHO-FD11 cells stably expressing FGE with different cell lines (FIG. 13B). In fact, FGE was processed after incubation with HEK293 cells but not with HeLa, HT1080 or immortalized MSD patient cells. In parallel, conditioned medium was incubated with CHO-FD11 cells to show that truncation of FGE does not occur due to the cultivation conditions used.

Further, increased incubation times with HEK293 cells led to increased level of Δ72-FGE in the conditioned medium (FIG. 13C). These data clearly show that FGE can be processed extracellularly by surface exposed proteases and this cleavage is cell type specific and time dependent. The cell type specificity may represent the furin expression level of the cell line or, at least, the amount of cell surface exposed and/or soluble furin.

### Example 16

### Processing of the N-Terminus Leads to Inactivation of FGE

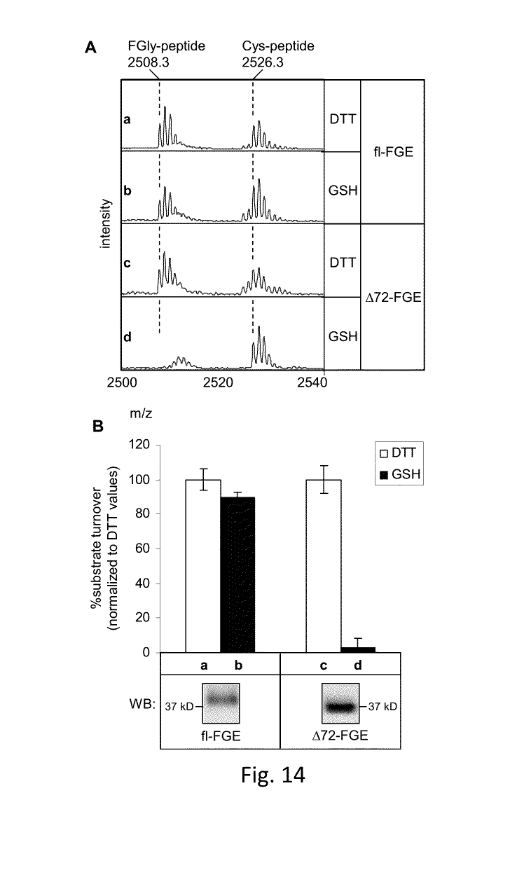

The inventors have recently shown that the N-terminal part of FGE (residues 34-68) is essential to activate sulfatases in vivo (12). By contrast, the N-terminal extension was not required for in vitro FGly-generating activity of purified secreted Δ72-FGE. However, this in vitro activity is dependent on the presence of the reductant dithiothreitol (DTT) (26, 32). To assess the physiological consequence of the N-terminal truncation by furin on the function of FGE, the inventors analyzed the FGly-generating activity of the processed and the unprocessed forms of secreted FGE in the presence of glutathione (GSH), a physiological reductant, or DTT serving as a control. The in vitro FGE activity assay (based on mass spectrometry, see Experimental Procedures) was performed with conditioned media obtained from CHO-FD11 cells expressing either FGE alone or coexpressing FGE plus furin leading to secretion of unprocessed FGE or processed Δ72-FGE, respectively (FIG. 14B, western blot panels). Reaction conditions (amount of FGE and incubation time) in presence of DTT were set to 50% turnover of the cysteine-containing substrate peptide (2526.3 m/z) to the FGly-containing product peptide (2508.3 m/z). The unprocessed FGE is active in the presence of both DTT and GSH as shown by the appearance of the product (2508.3 m/z) in the representative spectra (FIG. 14A, panels a and b), whereas processed FGE (Δ72-FGE) showed activity only in the presence of DTT but not when GSH was used as reductant (FIG. 14A, panels c and d). Quantitative analysis of the FGly-generating activity (% substrate turnover) of FGE and Δ72-FGE in the presence of GSH normalized to that of DTT (100%) revealed that the unprocessed form is as active with GSH as with DTT, but Δ72-FGE is barely active in the presence of GSH. These data clearly indicate that the FGly-generating activity of FGE with a physiological reductant is exclusively dependent on the presence of the N-terminal extension and furin-mediated processing functionally inactivates FGE during secretion. This agrees with the earlier observation that the forced expression of truncated FGE in MSD patient cells does not lead to intracellular sulfatase activation in vivo (12).

### Example 17

### Accessibility of the N-Terminus of FGE for Furin-Mediated Cleavage is Abolished when FGE is in Complex with ERp44

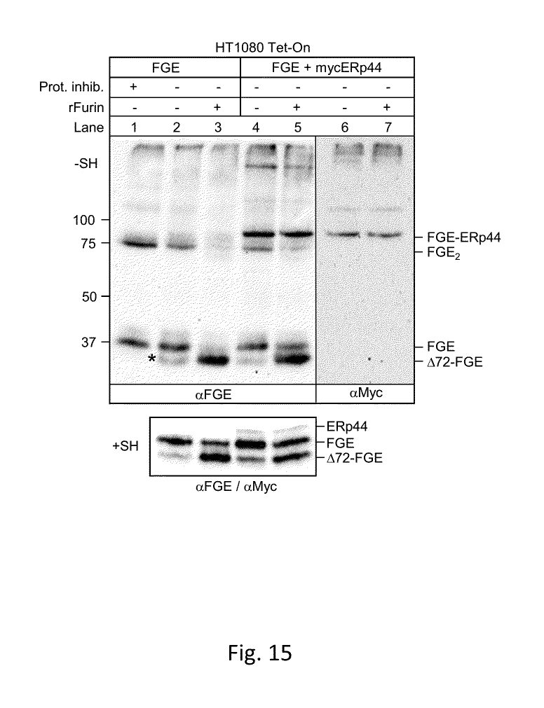

Although FGE is an ER resident protein, it lacks a canonical ER-retention motif. The inventors recently showed that FGE is retained in the ER by ERp44 via a thiol-independent mechanism, but nevertheless forms a disulfide-linked covalent complex with ERp44 through its N-terminal cysteines C50 and C52 (13). Interestingly, when co-expressed together with ERp44 lacking the RDEL sequence (ERp44ΔRDEL), a larger fraction of FGE was secreted, mainly in the full-length form. This indicates that FGE when secreted along with ERp44ΔRDEL as a complex escapes N-terminal trimming. In this study, using the in vitro furin cleavage assay, the inventors assessed furin-mediated processing of the FGE-ERp44 covalent complex (FIG. 8). Homogenates, pre-treated with NEM (to prevent post-lysis disulfide shuffling), from HT1080 cells expressing either FGE alone or FGE plus myc-ERp44 together from a bi-directional promoter were incubated with rFurin and analyzed by SDS-PAGE under non-reducing conditions. Upon overexpression, intracellular FGE via its N-terminal cysteines (C50 and C52) forms covalently cross-bridged homodimers (FIG. 15, lane 1 and 2) and, additionally, FGE-ERp44 heterodimers when co-expressed with ERp44 (FIG. 15, lane 4 and 6) as previously observed (13). Removal of the N-terminus by furin cleavage should lead to a disappearance of the homo-/hetero-dimeric FGE forms, which serves as an indicator for accessibility of the furin cleavage motif in FGE. Upon treatment with rFurin, the signals for monomeric as well as the homodimeric fraction of FGE decrease with a concomitant increase in the signal corresponding to the N-terminally truncated form (Δ72-FGE) (SEQ ID NO: 6), indicating that in both monomer and homodimer the furin-cleavage site is accessible to furin (FIG. 15, compare lanes 2 and 3; lanes 4 and 5). However, rFurin treatment does not abolish the covalent interaction between FGE and ERp44 as evidenced by the presence of an intact FGE-ERp44 heterodimer observed in untreated samples (FIG. 15, compare lanes 4 and 5; 6 and 7) and a quantitative increase of the unprocessed FGE form due to heterodimer formation when analyzed under reducing (+SH) conditions (FIG. 15, lanes 3 and 5).

These data show that the cleavage motif in the N-terminus of FGE is inaccessible when in complex with ERp44 and is thereby protected from furin cleavage.

### Example 18

### FGE as a Non-Canonical Substrate for Furin and Other PCs

In this study the inventors could show that furin-like PCs cleave the N-terminus of secreted FGE, with furin itself being the most effective and primary protease that performs this N-terminal processing. Several lines of evidence support our conclusion. FGE, like other proteins that are processed by PCs, bears a conserved cleavage site sequence containing the minimal consensus motif [R/K]-Xₙ-[R/K] [symbol] . Our phylogenetic analysis revealed that the RYSR motif of FGE is conserved in later diverging eukaryote species (FIG. 10); experimentally we show that this RYSR sequence represents an authentic processing motif, as alanine variants of the conserved arginines conferred resistance to cleavage. Moreover, the processing of FGE was abolished either when treated with RVKR-CMK, a peptide based inhibitor of PCs, or when expressed in CHO-FD11 cells that are deficient for furin; replenishing furin in these cells by transient expression led to a complete processing of secreted FGE. Expression of PACE4 or PC5a also led to FGE processing, albeit to a lesser extent compared to furin. Further, both intracellular and secreted FGE was processed by recombinant furin under in vitro conditions. All these data unambiguously lead to the conclusion that furin is the primary PC that mediates the processing of FGE during secretion.

The observation that this N-terminal processing occurs for endogenous secreted FGE signifies the physiological relevance of this processing event. Interestingly, a fraction (˜20-30%) of secreted FGE, independent of its expression level, is found in the unprocessed form. The escape from complete processing even at the very low amounts of endogenous FGE exiting the ER may reveal a kinetic limitation during the processing step and, at the same time, the importance of secreted fl-FGE. Since we observed an intracellular accumulation of FGE upon treatment with Brefeldin A indicating that an intact Golgi is necessary for secretion of FGE (our unpublished results), it is very unlikely that the fraction of the unprocessed form represents a population of FGE secreted via unconventional trafficking routes. Rather, we find that the processing is inefficient due to properties of the cleavage motif A classical furin cleavage motif is defined by the presence of arginines at P1 and P4 but residues in close vicinity of the cleavage site play an important role in the recognition and cleavage efficiency of PCs (33). The presence of a positively charged amino acid at P2 (mostly lysine or arginine) has been shown to improve the processing efficiency of furin, whereas no clear consensus has been found for residues at position P3. Interestingly, the presence of Ser at position P2 in some substrates has been suggested to be unfavorable for cleavage by mammalian furin and other PCs (29). Our mutational analysis of Tyr70 and Ser71 residues indicates that this holds true also for FGE. Modifying the residues at positions P2 and P3 to positively charged residues, thus representing a more favorable motif, led to a strongly increased efficiency of processing with a maximum effect observed by mutating Ser71. Thus, the presence of Ser at position P2 is the major cause for inefficient processing of FGE. Interestingly, Tyr70 and Ser71 are absolutely conserved throughout evolution to a degree even higher than the essential basic (arginine) residues, supporting our conclusion that the RYSR motif in FGE is a non-canonical cleavage motif that evolved to confer suboptimal cleavage efficiency.

Why should FGE be Processed at all?

Both phylogenetic comparison and experimental data clearly demonstrate that the FGE protein during evolution gained a function by addition of the N-terminal extension harboring the Cys-Gly-Cys sequence. This motif is critical for the biological activity of eukaryotic FGE (12) and fully conserved from human to sponge. The N-terminal extension is found only in eukaryotes (encoded by exon 1) and the fact that eukaryotic FGly generation is a co-translational event occurring in a specialized compartment, the ER, suggests that the N-terminal extension serves to adapt FGE to ER-based functioning in eukaryotes. One of the gained properties is ER retention through interaction of ERp44 with the N-terminus (13), another seems to be related to inter- or intramolecular activation of the catalytic domain of FGE by the Cys-Gly-Cys motif (12). Further ER-specific aspects, such as competence to act on nascent sulfatase polypeptides emerging at ER import sites, might be associated with the N-terminal extension.

Interestingly, prokaryotic FGE from Streptomyces coelicolor, which lacks the N-terminal extension, when expressed in the cytoplasm of eukaryotic cells was shown to possess FGly-generating activity acting on engineered cytosolic model substrates containing the FGly-modification signature (34, 35). On the other hand, we have shown that N-terminally truncated human FGE when expressed in the ER, did not possess FGly generating activity, which agrees with the hypothesis that N-terminal processing irreversibly abrogates ER-based FGE functioning. On the basis of these two observations one can speculate that furin-mediated removal of the N-terminus during secretion could serve a possible mechanism in later diverging eukaryotes to generate FGE that is active under extracellular conditions. However, an extracellular formylglycine generation has not been reported so far.

### Processing by PCs as a Means of Enzyme Inactivation

Our data indicate that a direct consequence of N-terminal processing of secreted human FGE is its inactivation. The N-terminal extension was found to be essential for in vitro activity in the presence of glutathione, a physiological reductant, which does not sustain activity of N-terminally truncated FGE. This corroborates our previous observation that the N-terminus is essential for biological activity in cultured cells. Since removal of this part renders secreted FGE inactive, we propose that the observed furin-mediated processing during secretion is a physiological means for regulation of FGE function/activity. The need for inactivation of the enzyme is not clear, but it might be a mechanism necessary to avoid generation of toxic aldehydes in extracellular or cell surface proteins in case that FGE evades its indirect ERp44-mediated ER retention.

Proteolytic processing mediated by PCs along the secretory pathway is a widely used necessary step in the activation or maturation of many proteins that are involved in various cellular processes. In contrast, inactivation of a protein function by furin-mediated processing only recently has been recognized as a novel mode of regulation, as shown for PCSK9 (36). The function of PCSK9, in mediating LDL-receptor internalisation and degradation, was shown to be inactivated by furin-catalyzed cleavage of PCSK9. FGE could represent another protein that is inactivated by proteolytic processing mediated by PCs. Nevertheless, one cannot exclude the possibility that the secreted N-terminally truncated FGE could perform as yet unknown extracellular functions other than formylglycine generation.

### Incomplete Processing as a Means to Provide Active FGE to the Extracellular Space?

On the other hand, it is tempting to assume that the inefficient processing could represent a functional tool for regulated release of FGE in the unprocessed active form. The low but significant secretion of fl-FGE even under endogenous expression levels might indicate such a regulated release of active FGE. In fact, it has been shown that secreted FGE can enter other cells by mannose receptor-mediated internalization and activate sulfatases in a paracrine manner after reaching the ER of the recipient cells (16). To exert this function, FGE should escape furin-mediated inactivation during secretion. As a proof of principle our data show that FGE, when in complex with ERp44, is barred from furin processing. This indicates that masking the cleavage site by an interacting protein during secretion could significantly hinder recognition by furin and could lead to the secretion of FGE in the unprocessed form. Alternatively, a population of FGE destined to be secreted in the active form could have an altered conformation wherein the cleavage site is masked due to intramolecular interactions of the N-terminus to the core of the protein, thus preventing truncation. In combination with the suboptimal cleavage motif, an impaired processing by furin due to an inaccessible cleavage site (either by inter- or intramolecular interactions) could further increase the fraction of unprocessed secreted FGE. The secretion of FGE (14, 15) and possible re-uptake by other cells (16) has been shown to be a multistep regulated process and our findings extend this complex regulation of FGE function to an additional level.

### Perspective

The finding that FGE is secreted in the unprocessed form by furin-deficient CHO cells should pave the way for production and detailed in vitro analysis of fl-FGE, which hopefully will lead to a more complete understanding of the structure-function relationship of FGE. It should further allow studying the cell biology of FGE in more detail in order to address the questions and concepts put forward in this study. One of the concepts to be tested involves uptake of recombinant fl-FGE by recipient cells, which if true, might be developed into strategies for enzyme-replacement therapy in MSD patients.

TABLE 2
N-terminal FGE amino acid sequences used for WebLogo generation.

| Abbr. | Species | Classification | Sequence P8-P8′ | number of sequences |
|---|---|---|---|---|
| homSap | homo sapiens | euarchontoglires | AAAHRYSREANAPGPV | SEQ ID NO: 45 |
| panTro | pan troglodytes | | AAAHRYSREANAPGPV | SEQ ID NO: 45 |
| gorGor | gorilla gorilla | | AAAHRYSREANAPGPV | SEQ ID NO: 45 |
| ponAbe | pongo abelii | | AAAHRYSREANAPGPV | SEQ ID NO: 45 |
| nomLeu | nomascus leucogenys | | AAAHRYSREANAPGPV | SEQ ID NO: 45 |
| rheMac | macaca mulatta | | GAAHRYSREANAPGSV | as SEQ ID NO: 45, |
| macFas | macaca fascicularis | | GAAHRYSREANAPGSV | wherein A₁ is G and P₁₅ |
| papHam | papio hamadryas | | GAAHRYSREANAPGSV | is S |
| calJac | callithrix jacchus | | AAAHRYSREANAPGPV | SEQ ID NO: 45 |
| tarSyr | tarsius syrichta | | AAAHRYSREANAPGPF | as SEQ ID NO: 45, |
| otoGar | otolemur garnettii | | AAAHRYSREANAPGPI | wherein V₁₆ is F or I |
| tupBel | tupaia belangeri | | AAAHRYSREANVPGPV | EQ ID NO: 45, wherein |
| | | | | A₁₂ is V |
| musMus | mus musculus | | AAAQRYSREANAPGLT | SEQ ID NO: 52 |
| ratNor | rattus norvegicus | | AAAQRYSREANAQGLT | As SEQ ID NO: 52, |
| | | | | wherein P₁₅ is Q |
| speTri | spermophilus tridecemlineatus | | SAAHRYSREANAPSAL | SEQ ID NO: 53 |
| dipOrd | diplomys ordii | | AAAHRYSREANAPPGP | SEQ ID NO: 54 |
| oryCun | oryctolagus cuniculus | | AAAHPYSREANAPGPV | as SEQ ID NO: 45, |
| | | | | wherein R₅ is P |
| ochPri | ochotona princeps | | AAAHLYSREANAPGPV | as SEQ ID NO: 45, |
| | | | | wherein R₅ is L |
| canFam | canis familiaris | laurasiatheres | AAAHRYSREANAPGQV | SEQ ID NO: 45, |
| felCat | felis catus | | AAAHRYSREANAPGQV | wherein P is Q |
| ursAme | ursus americanus | | AAAHRYSREANAPGQV | |
| ailMel | ailuropoda melanoleuca | | AAAHRYSREGNAPGQV | as SEQ ID NO: 45, |
| | | | | wherein A₁₀ is G an P₁₅ |
| | | | | is Q |
| pteVam | pteropus vampyrus | | AAAHRYSREANAAGPG | as SEQ ID NO: 45, |
| | | | | wherein P₁₂ is A and |
| | | | | V₁₆ is G |
| turTru | tursiops truncatus | | AAAHRYSREANAPGSV | as SEQ ID NO: 45, |
| | | | | wherein P₁₅ is S |
| susScr | sus scrofa | | AAAQQYSREANAPGPV | SEQ ID NO: 55 |
| equCab | equus caballus | | AAAHRYSREANAPGSG | as SEQ ID NO: 45, |
| | | | | wherein P₁₅ and V₁₆ is |
| | | | | SG |
| bosTau | bos taurus | | AAAHRYSREANAPGSV | as SEQ ID NO 45, |
| capHir | capra hircus | | AAAHRYSREANAPGSV | wherein P₁₅ is S |
| oviAri | ovis aries | | AAPHRYSREANAPGSV | |
| eriEur | erinaceus europae | | AAAHRYSWEANAPGPD | SEQ ID NO: 56 |
| loxAfr | loxodonta africana | atlantogenata | AAAHRYSREANVPGPV | as SEQ ID NO 45, |
| mamPri | mammuthus primigenius | | AAAHRYSREANVPGPV | wherein A₁₂ is V |
| proCap | procavia capensis | | AAAHRYSREANVPGPV | |
| echTel | echinops telfairi | | AAAHRYSREANAPGLG | as SEQ ID NO: 45, |
| | | | | wherein PV₁₆₋₁₇ are LG |
| dasNov | dasypus novemcinctus | | AAAHGYSREANAQGRA | SEQ ID NO: 57 |
| choHof | choloepus hoffmanni | | AAAHRYSREANAPGRA | as SEQ ID NO: 45, |
| | | | | wherein PV₁₆₋₁₇ are RA |
| monDom | monodelphis domestics | marsupials | SAAHRYSREANVAEPA | SEQ ID NO: 58 |
| macEug | macropus eugenii | | SAAHKYSREANVAERA | SEQ ID NO: 59 |
| anoCar | anolis carolinensis | early diverging | VASRKYSREVHLPQQP | SEQ ID NO: 60 |
| galGal | gallus gallus | amniotes | VATVRYSAAANDGRSP | SEQ ID NO: 61 |
| melGal | meleagris gallopavo | | AAARRYSAVANGGRSS | SEQ ID NO: 62 |
| allMis | alligator mississippiensis | | AAVRRYSPEANAQRPG | SEQ ID NO: 63 |
| pytMol | python molurus | | VAARKYSLDANVSQQP | SEQ ID NO: 64 |
| ambMex | ambystoma mexicanum | | HRAARYSREANEPLKA | SEQ ID NO: 65 |
| xenTro | xenopus tropicalis | | DSPHKYSREANEPEPA | SEQ ID NO: 66 |
| xenLae | xenopus laevis | | ENSHKYSREANEPEPT | SEQ ID NO: 67 |
| takRub | takifugu rubripes | ray-finned fish | VDGAKYSRGASRRDQT | SEQ ID NO: 68 |
| tetNig | tetraodon nigroviridis | | EPGPKYSRGANGRDED | SEQ ID NO: 69 |
| danReg | danio rerio | | DVNRIYSKTANEGPDD | SEQ ID NO: 70 |
| oncMyk | oncorhynchus mykiss | | KESSKYSKKSNERHTD | SEQ ID NO.: 71 |
| salSal | salmo salar salmon | | KESSKYSKKSNERHTD | SEQ ID NO.: 71 |
| oryLat | oryzias latipes | | MTEPKYSSAGSKSNGG | SEQ ID NO.: 72 |
| ictPun | ictalurus punctatus | | DEDGKYSERANKEFVG | SEQ ID NO.: 73 |
| ictFur | ictalurus furcatus | | DEDGKYSERANKEFVG | SEQ ID NO.: 74 |
| gasAcu | gasterosteus aculeatus | | EEGSKYSEGANGRFVQ | SEQ ID NO: 75 |
| oreNil | oreochromis niloticus | | LDEDKYSKDANDRTNQ | SEQ ID NO: 76 |
| osmMor | osmerus mordax | | SHASKYLQTTNEKPTL | SEQ ID NO: 77 |
| esoLuc | esox lucius | | RESGIYSKTSNEKLTD | SEQ ID NO: 78 |
| cioInt | ciona intestinalis* | urochordaes | EVAEEPDLPLQKVSSD | SEQ ID NO: 79 |
| cioSav | ciona savignyi* | | EVEEEPDIPLPTIPTG | SEQ ID NO: 80 |
| halRor | halocynthia roretzi* | | MDQYEVTENAEQHLVE | SEQ ID NO: 81 |
| oikDio | oikopleura dioica | | AGTFTMGDNEELMPGD | SEQ ID NO: 82 |
| braFlo | branchiostoma floridae* | cephalochordates | PVEGEGGAEAPEFDKD | SEQ ID NO: 83 |
| strPur | strongylocentrotus purpuratus* | echinoderms | ALEEKYSREANDPIDH | SEQ ID NO: 84 |
| parLiv | paracentrotus lividus | | PLALKYSKEVNDATGS | SEQ ID NO: 85 |
| sacKow | saccoglossus kowalevskii* | hemichordates | KSGGEVNDHHGVEQHD | SEQ ID NO: 86 |
| droMel | drosophila melanogaster | ecdysozoa | SGQVCQQRAQGAHSHY | SEQ ID NO: 87 |
| anoGam | anopheles gambiae | | KERVIFPTDAAQHSPS | SEQ ID NO: 88 |
| mayDes | mayetiola destructor | | SNENKDDSSNEMCSNP | SEQ ID NO: 89 |
| bomMor | bombyx mori | | LYSGNNNEQCSVENIS | SEQ ID NO: 90 |
| apimel | apis_mellifera | | YKKEIQDSCLANDILH | SEQ ID NO: 91 |
| camFlo | camponotus floridanus | | GYCVIDNSKFDAIDIN | SEQ ID NO: 92 |
| acyPis | acyrthosiphon pisum | | VCTSSATSNSERLDSE | SEQ ID NO: 93 |
| rhoPro | rhodnius prolixus | | CIPSSFLDLLKQTREN | SEQ ID NO: 94 |
| pedHum | pediculus humanus | | KVNFKDDAIFEEQEIS | SEQ ID NO: 95 |
| triCas | tribolium castaneum | | EPHNKYSKTFNEGGDS | SEQ ID NO: 96 |
| denPon | dendroctonus ponderosae | | NPSQKYKRDLNENPAN | SEQ ID NO: 97 |
| lepDec | leptinotarsa decemlineata | | NPSHKYMKESNEETGN | SEQ ID NO: 98 |
| eriSin | eriocheir sinensis | | SPETSPVLENNEESPN | SEQ ID NO: 99 |
| ambVar | amblyomma variegatum | | GSTSDDDEESRVEVED | SEQ ID NO: 100 |
| rhiMic | rhipicephalus microplus | | NHDEKARLDSENAALN | SEQ ID NO: 101 |
| aplCal | aplysia californica | lophotrochozoa | SQAPESSDPSGSVGVD | SEQ ID NO: 102 |
| lotGig | lottia gigantea | | KGSEQADDKSGMYHPQ | SEQ ID NO: 103 |
| nemVec | nematostella vectensis | cnidarians | KKFVKYSKKANVDQDI | SEQ ID NO: 104 |
| acrMil | acropora millepora | | KITDRKNGEGKFNLKS | SEQ ID NO: 105 |
| monFav | montastraea faveolata | | MTLNTGNTDGEIKLKS | SEQ ID NO: 106 |
| plePil | pleurobrachia pileus | ctenophores | KISDMKRNEQQSEHPN | SEQ ID NO: 107 |
| ampQue | amphimedon queenslandica | basal metazoan | SVEKEESEEPKAQAEE | SEQ ID NO: 108 |

The 88 species used are listed and classified. The N-terminal FGE sequences were centered at P1 (Arg72 of human FGE) or based on a ClustalW alignment for group III that do not bear a R/K/X-Y-S-R/K/X motif (SEQ ID NO: 44). Sequences are given for the part that corresponds to the cleavage site P8-P8′ and the core region of P4-P1 is marked in bold letters.* intronated differently.

### Example 19

### Biochemical Characterization and Optimization of the Activity and Stability of Recombinant fl-FGE-R69A/R72A and Δ72-FGE Produced with Insect Cells

To provide a high protein quality (e.g. stability and enzymatic activity) of recombinant fl-FGE-R69A/R72A and Δ72-FGE-wt produced from insect cells as described above, the following biochemical parameters were optimized: concentration of the reducing agents DTT and glutathione (GSH), pH, and protein stability upon storage.

1. Separation of the Recombinant fl-FGE-R69A/R72A Monomer and Dimer by Size-Exclusion Chromatography

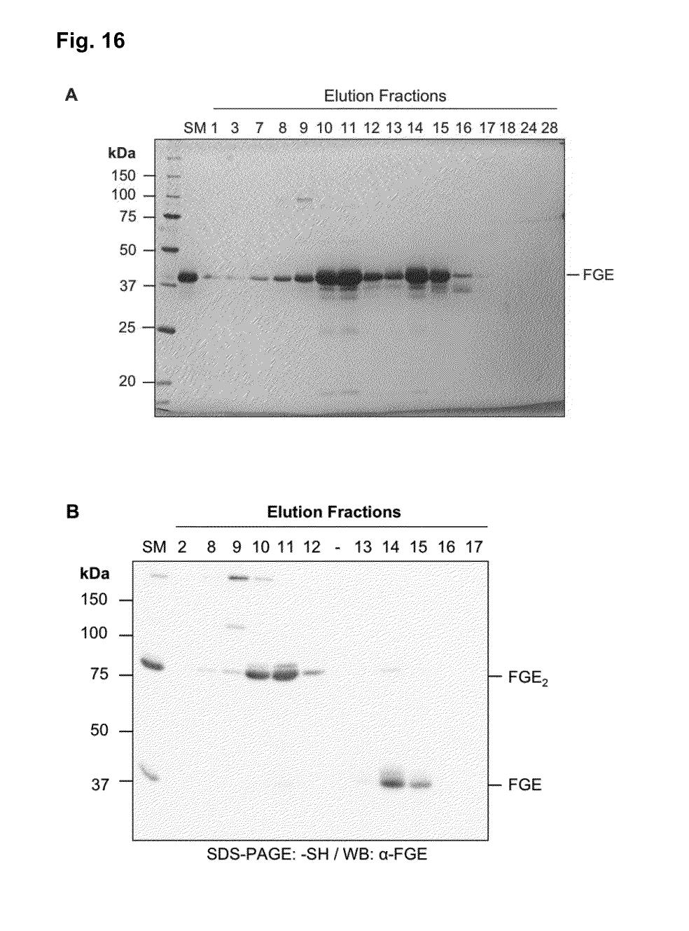

In an attempt to remove imidazole from Ni-NTA affinity purified fractions containing fl-FGE-R69A/R72A (see Example 5), a second step purification using size-exclusion chromatography was employed. Pooled Ni-NTA elution fractions (2.45 mg/ml) were concentrated using centricon filters (Corning B.V. Lifesciences) and purified in a Superdex 200 column. Analysis of the elution fractions (about 3 mg/ml) by SDS-PAGE (10%) and coomassie staining showed that purified fl-FGE-R69A/R72A was found in fractions 9-15 (FIG. 16A). Interestingly, the major amount of FGE was concentrated in two sets of fractions (10-11 and 14-15) indicating two different population of FGE differing in size. To gain insights on the nature of this distribution and to determine the identity of these two fractions, the elution fractions were analyzed by SDS-PAGE under non-reducing conditions followed by decorating the western blot membrane with FGE anti-serum (FIG. 16B). Clearly, major amount of fl-FGE-R69A/R72A was observed in fractions 10-11 and 14-15 (compare FIG. 16A). However, in fractions 10-11, FGE was observed running at the molecular size above 75 kDa, suggestive of a dimeric form whereas fractions 14-15 represented the monomeric form. Note that both the monomeric and dimeric forms (and minor fraction of oligomeric forms as well) are present in the starting material (SM, FIG. 16B), which clearly get separated after size exclusion chromatography yielding a homogenous preparation.

The dimer is sensitive to DTT or GSH or any reducing agent suggesting that it is disulfide-mediated and that this disulfide-bridged dimer is dependent on the presence of the N-terminal domain and mediated by cysteine residues C50 and C52.

In summary, using size exclusion chromatography we were able to separate monomeric and dimeric FGE to homogeneity.

2. Imidazole Efficiently Stabilizes Recombinant fl-FGE-R69A/R72A

Purified enzymes are preferably stored frozen at −20° C. or −80° C., but it is known that the freeze-thaw cycle can lead to a decrease in the stability of proteins and/or accompanied loss of enzymatic activity. To minimize this effect, it is common to add stabilizing agents to preserve the stability and functionality of proteins during long-term storage. Analyses of conditions that affect the stability of purified recombinant fl-FGE-R69A/R72A and Δ72-FGE-wt were performed. The stability was assessed by analyzing the purified protein by SDS-PAGE and subsequently visualized by Coomassie staining or western blotting using FGE anti-serum as described above. In the case of Δ72-FGE-wt, analysis of the protein after thawing from −80° C. and short-term storage at 4° C. did not lead to any change in the molecular integrity indicating a high stability (data not shown).

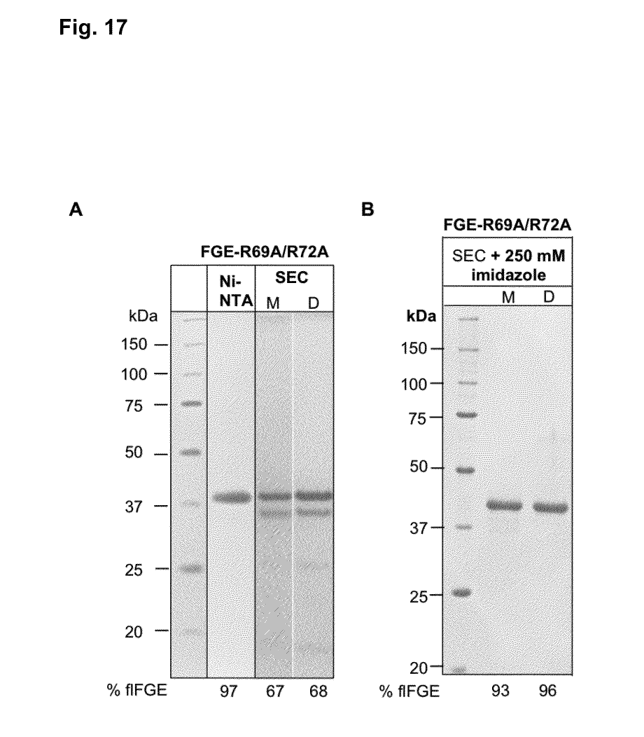

In the case of fl-FGE-R69A/R72A, the purified fractions from Ni-NTA affinity chromatography (containing 250-500 mM imidazole), when analyzed after long-term storage in −80° C., were highly stable. However, generation of a truncated fragment (less than 5% of the total protein), with a molecular weight of ˜37 kDa, was observed after thawing (Panel Ni-NTA, FIG. 17A). Addition of commonly used protease inhibitors namely PMSF, Protease inhibitor cocktail (from Sigma) and Pefa block did not prevent the generation of this 37 kDa fragment (not shown).

Generation of truncated FGE was increasingly detected upon removing the imidazole by size exclusion chromatography (Panel SEC, FIG. 17A). To gain further insights, aliquots of the purified fraction from size exclusion chromatography were incubated at 4° C. for either 3.5 h or 20 h in the presence of various additives like NaCl, imidazole, arginine and glycerol and later assessed by western blot using FGE anti-serum. Prolonged incubation at 4° C. led to generation of truncated fragments. However, addition of 250 mM imidazole or 850 mM NaCl prevented further increase of FGE truncation (not shown).

These data indicated that imidazole serves as a stabilizing agent for recombinant fl-FGE-R69A/R72A. Accordingly, addition of 250 mM imidazole to fractions containing monomeric or dimeric FGE that were obtained after size exclusion chromatography were more stable, as assessed after one freeze-thaw cycle including storage for two weeks at −80° C. (FIG. 17B). Since these fractions, that was stored with imidazole and used for biochemical studies (see below), were fully functional, thus indicating that the presence of imidazole did not negatively affect the activity of recombinant FGE.

In summary, a high fl-FGE-R69A/R72A protein quality is conserved by storage as purified protein solution in frozen state at −80° C. Addition of imidazole (e.g. about 250 mM) maintains the protein stability upon (long-term) storage of purified recombinant fl-FGE-R69A/R72A.

3. Optimal In Vitro Activity of Recombinant fl-FGE-R69A/R72A and Δ72-FGE-Wt Shows Different Dependency on DTT and GSH

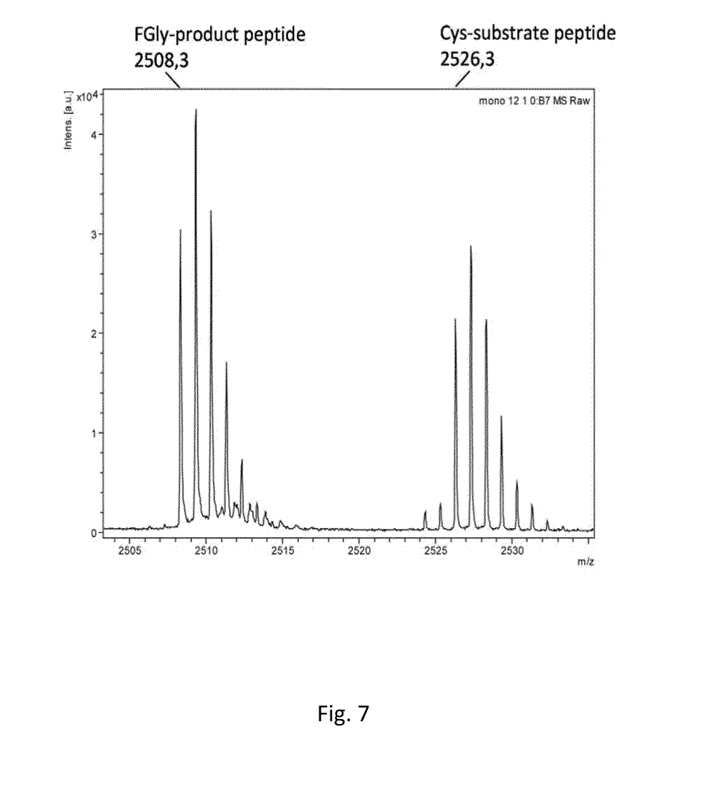

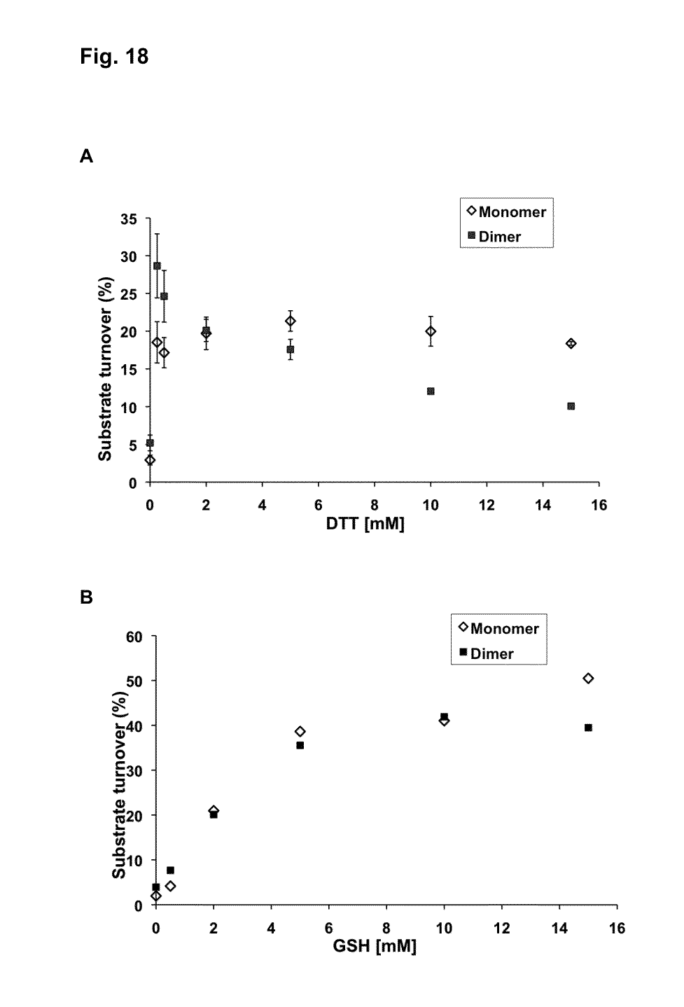

The dependence of FGly-generating activity of recombinant fl-FGE-R69A/R72A on the reducing agents DTT and GSH were analyzed at pH 9.3 under standard assay conditions (FGE and substrate peptide were incubated at 37° C. in 50 mM Tris/HCl, 67 mM NaCl, and 0.33 mg/ml BSA in 30 μl volume. The reaction was started by addition of substrate) using a MALDI-mass spectrometry based in vitro activity assay (Ennemann et al., 2013 and FIG. 7). Recombinant fl-FGE-R69A/R72A (either monomer or dimer) was incubated with the substrate peptide under various concentrations (0-15 mM) of either DTT or GSH and the activity (percent ratio of the signal intensities of product (FGly-containing peptide) to substrate (Cys-containing peptide) were analyzed (FIG. 18). In the presence of DTT, monomeric FGE showed a DTT-dependent increase in the activity, with maximal activity observed at 2 mM DTT Whereas dimeric FGE showed a showed a sharp increase in activity at lower concentrations of DTT with a maximal activity with 0.1-1 mM DTT (FIG. 18A). However, for both forms of FGE at DTT concentrations above 10 mM a decrease in the activity was observed indicating an inhibitory effect with dimeric FGE showing the highest sensitivity. When analyzed in the presence of GSH, both monomeric and dimeric FGE showed a GSH-concentration dependent increase in the activity with the maximal activity observed at ≧5 mM GSH (FIG. 18B). Of note and in contrast to DTT, FGE exhibited a typical hyperbolic increase in activity with increasing concentrations of GSH.

In conclusion, the data show that fl-FGE-R69A/R72A exhibited, albeit very low activity in the absence of externally added reducing agents, a DTT/GSH dependent increase in the activity. Optimal activity under in vitro conditions is achieved with 1-10 mM DTT for monomeric FGE, 0.1-2 mM DTT for dimeric FGE and 5-15 mM GSH for both the forms. To summarize, fl-FGE-R69A/R72A from insect cells is fully functional in the presence of either DTT or glutathione (GSH) as reducing agent, while activity of Δ72-FGE-wt strictly relies on DTT (see FIG. 14).

4. Optimal pH Conditions for In Vitro Activity of Recombinant fl-FGE-R69A/R72A and Δ72-FGE-wt

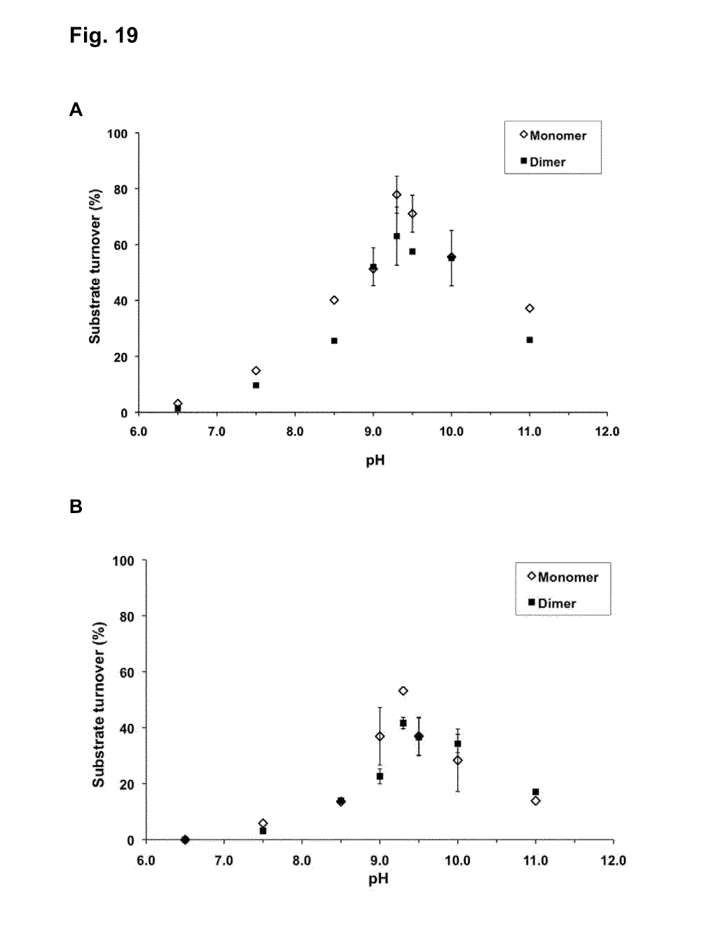

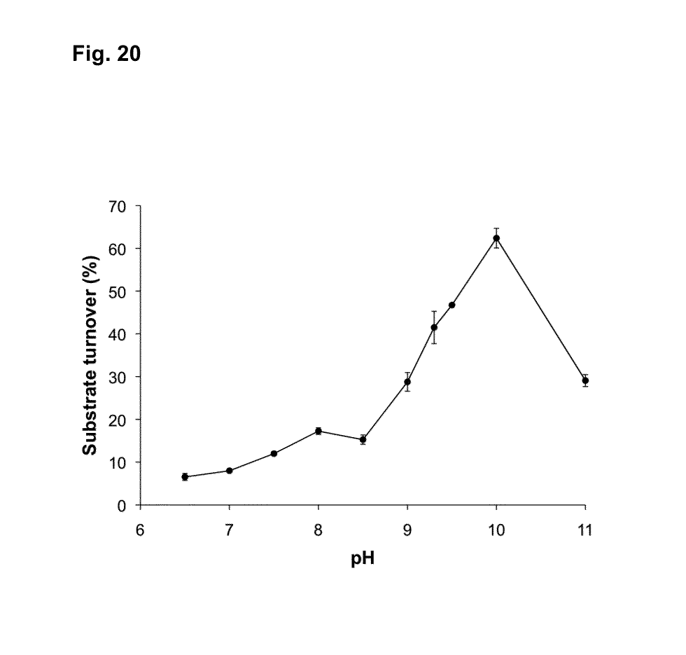

The pH-dependence for in vitro activity of recombinant fl-FGE-R69A/R72A and Δ72-FGE-wt was analyzed under optimal DTT and GSH concentrations. Accordingly, the activity of recombinant fl-FGE-R69A/R72A in the presence of 2 mM DTT, under standard assay conditions, was measured in buffers of pH ranging from pH 6.5 to 11.0 (FIG. 19A). The maximal activity in the presence of 2 mM DTT was observed at pH 9.3 with an activity range from pH 7.5 to 11. In the presence of 5 mM GSH, the maximal activity was also observed at pH 9.3 and a very similar activity profile ranging from pH 7.5 to 11 (FIG. 19B). When measuring the pH-dependence of Δ72-FGE-wt in the presence of 2 mM DTT (FIG. 20), the enzyme was highly active in a pH range of 9 to 11 showing an optimum at pH ˜10. In summary, fl-FGE-R69A/R72A requires less alkaline conditions for optimum activity than Δ72-FGE-wt.

### Example 20

### “In Vitro” (Cell-Free) Conversion and Modification of a Therapeutically Active Protein Under Physiological Conditions

Physiological reductants like GSH, a milder reducing agent, are favorable to use than a strong reducing agent like DTT for generation of aldehyde-tag in disulfide containing proteins or peptides, in particular therapeutic or diagnostic proteins such as antibodies, growth hormones or vaccines for instance. The presence of a stronger reducing agent could lead to uncontrollable and unfavorable reduction of disulfide bridges that might be crucial for the structural stability and homogeneity and in turn potentially affect the function or side effects of the protein/biopharmaceutical. Cells producing monoclonal IgG antibodies obtained from ATCC catalog no: CRL-1716) can be used as an example of a therapeutically active protein. These cells can be transfected with a plasmid coding for the 23 aa long peptide of Example 8 (SEQ ID NO: 46) according to standard protocols in order to produce IgG antibodies exhibiting an C-terminal aldehyde tag. As an alternative only the minimized 6-residue sequence LC×P×R can be fused to the IgG protein as described by Wu et al, 2009, Proc Natl Acad Sci USA 106: 3000-3005.

16 ng of expressed IgG-aldehyde tag proteins can be incubated with 12 ng of FGE-R69A/R72A for 20 min up to overnight at 20 under assay conditions described in Example 8, wherein 5 mM GSH instead of DTT is used as a reducing agent.

The therapeutically active protein of interest can be purified from this reaction mixture by standard methods like affinity purification or size exclusion chromatography. The efficiency of conversion after proteolytic digestion of the therapeutically active protein, can be analyzed by MALDI-Tof mass spectrometry assay of peptides. For this purpose, an aliquot of the reaction mixture can be treated with denaturing agents like urea (4-8 M) or guanidine hydrochloride (up to 6 M) to stop the reaction and then diluted in protease (for example trypsin) buffer (20 mM Tris/C1, pH 8.0-8.6). The protein can be digested by addition of trypsin in the ratio of 1:20 (protease:protein) and overnight incubation at 37° C. The protease reaction can be stopped by adding 3 μL of 20% trifluoroacetic-acid (TFA), immediately followed by vortexing and by a short centrifugation at 10000×g. The peptides can be purified and concentrated by C18-Zip-Tip treatment. Therefore the Zip-Tip can be prepared by pipetting three times 10 μL of 50% acetonitrile, 0.05% TFA in water and three times 10 μL 0.1% TFA in water. The bound IgG protein can be washed by pipetting 10 times 10 μL 0.1% TFA in water and eluted in 10 μL 50% acetonitrile, 0.05% TFA in water by pipetting 10 times up and down. For MS-analysis the following matrix can be freshly prepared: 40 μL of a saturated α-cyano-hydroxycinnamic acid solution in acetone can be added to 10 μL of a solution containing 10 mg/mL nitrocellulose in 50% acetone/50% isopropanol (v/v). 0.5 μL of the matrix can be spotted onto a polished steel target and 1 μL of the purified sample can be added. The dried sample spot can be analyzed by MALDI-ToF mass spectrometry using the UltrafleXtreme spectrometer from Bruker Daltonics. The cysteine containing substrate peptide and the FGly containing product peptide can be detected.

Subsequently the isolated proteins can be further outfitted with an aldehyde group for site-specific chemical modification with aminooxy- or hydrazide-functionalized moieties, including fluorophores, affinity tags, and PEG chains according to standard methods, see for further information U.S. Pat. No. 6,570,040, U.S. Pat. No. 6,214,966 as well as WO2012097333 which are incorporated herein by reference.

Accordingly, an aldehyde tag at a predetermined site can be provided by genetic engineering into a therapeutically active protein.

### Example 21

### Therapeutic Use of FGE for Treatment of Specific Lysosomal Storage Disorders (LSDs): Gaucher Disease and Multiple Sulfatase Deficiency (MSD)

### Gaucher Disease

Twelve patients with non neuronopathic type 1 Gaucher's disease can be selected for participation in the trial from among patients referred to the Developmental and Metabolic Neurology Branch of the National Institute of Neurological Disorders and Stroke. The diagnosis can be confirmed by assaying glucocerebrosidase activity in extracts of cultured skin fibroblasts. Patients are required to be at least six years old and to have an intact spleen; they could be of either sex. The hemoglobin level at the time of entry into the study has to be less than 110 g per liter. All participants should be serologically nonreactive for hepatitis B surface antigen and human immunodeficiency virus (HIV) and should have no evidence of intercurrent cardiopulmonary, renal, infectious, or neoplastic disease. A complete series of vaccinations against poliovirus is required of all participants, as a negative pregnancy test of all female patients of childbearing age see also Barton et al., N Engl J Med 1991; 324:1464-1470.

1 mg/ml of an isolated purified polypeptide of this invention having the sequence of SEQ ID NO:4, SEQ ID NO: 8 and SEQ ID NO: 26 (corresponding to fl FGE variants described above) can be formulated into a composition buffer (10 mM citrate, 140 mM NaCl, 10 mM succinate, 140 mM NaCl, pH 10 mM succinate, 140 mM NaCl, 10 mM histidine, 140 mM NaCl, and 10 mM glycylglycine, 140 mM NaCl, pH 8.0).

Subsequently purified fl FGE recombinant enzyme (at a dose related to kilogram body weight) formulated into the composition buffer can be injected intravenously at a dose of per kilogram of body weight once weekly for 52 weeks to the patient. For each infusion, the requisite amount of enzyme can be diluted to a total volume of 100 ml with 0.9 percent sodium chloride solution (U.S.P.). To guard against adverse reactions, each patient can be given a test dose of 5 ml and observed for 10 minutes. The remainder of the dose can then be infused over a period of one to four hours.

A complete blood count including a reticulocyte count, routine serum biochemical values, serum acid phosphatase activity, the prothrombin and partial-thromboplastin times, and the plasma glucocerebroside level can be determined before enzyme infusion. Plasma glucocerebroside levels can be quantified by high-performance liquid chromatography. Infusions can be continued without interruption for a minimum of nine months. Routine urinalyses can be performed, and serum specimens can be analyzed for the presence or absence of antibody to the infused enzyme every three months. Chest radiography, electrocardiography, testing for hepatitis B and HIV, radiography of the long bones, and quantitative abdominal magnetic resonance imaging can be repeated at six-month intervals.

Serial analyses of the hemoglobin concentrations, platelet count, serum acid phosphatase activity, plasma glucocerebroside level, and hepatic and splenic volumes can serve as markers of the clinical response to enzyme infusions. Changes in the skeleton can be monitored radiographically.

### Multiple Sulfatase Deficiency (MSD)

SUMF1 mutations in patients with a neonatal very severe course of disease are either nonsense mutations with large deletions, frameshift mutations or missense mutations directly affecting the active site of FGE (like p.C336R).

1 ml blood sample obtained from a patient can be centrifuged at 100 rpm for 5 min and cells can be resuspended in a Tris buffer, pH 7.2. DNA is subsequently isolated according to QIAamp DNA Blood Mini Kit® manufacture instructions (Qiagen, Hilden, Germany). Genomic DNA can be tested for the presence of FGE missense mutations found in homozygosity (or in combination with a frame-shift null allele) in MSD patients (p.A177P, p.W179S, p.A279V, p.R349W). For conformation purpose, FGE protein can be isolated from fibroblasts of a patient's sample and can be further analyzed as described in Harmatz et al., Acta Paediatr Suppl. 2005 March; 94(447):61-8; discussion 57; Kakkis et al., N Engl J Med. 2001 Jan. 18; 344(3):182-8, in order to confirm that the FGE protein shows defects or decreased stability.

Subsequently purified fl FGE recombinant enzyme formulated into a composition, (see section Gaucher's disease above) at a dose of purified fl FGE recombinant variant of body weight (such as 100 units/g) can be given intrahecal or intravenously once weekly for 52 weeks to the patient.

### EQUIVALENTS

Those skilled in the art will recognize, or be able to ascertain using no more than routine experimentation, many equivalents to the specific embodiments of the invention described herein. The scope of the present invention is not intended to be limited to the above Description, but rather is as set forth in the appended claims. The invention includes embodiments in which exactly one member of the group is present in, employed in, or otherwise relevant to a given product or process. The invention also includes embodiments in which more than one, or all of the group members are present in, employed in, or otherwise relevant to a given product or process. Furthermore, it is to be understood that the invention encompasses variations, combinations, and permutations in which one or more limitations, elements, clauses, descriptive terms, etc., from one or more of the claims is introduced into another claim dependent on the same base claim (or, as relevant, any other claim) unless otherwise indicated or unless it would be evident to one of ordinary skill in the art that a contradiction or inconsistency would arise. Where elements are presented as lists, e.g., in Markush group or similar format, it is to be understood that each subgroup of the elements is also disclosed, and any element(s) can be removed from the group. It should it be understood that, in general, where the invention, or aspects of the invention, is/are referred to as comprising particular elements, features, etc., certain embodiments of the invention or aspects of the invention consist, or consist essentially of, such elements, features, etc. For purposes of simplicity those embodiments have not in every case been specifically set forth herein. It should also be understood that any embodiment of the invention, e.g., any embodiment found within the prior art, can be explicitly excluded from the claims, regardless of whether the specific exclusion is recited in the specification.

It should also be understood that, unless clearly indicated to the contrary, in any methods claimed herein that include more than one act, the order of the acts of the method is not necessarily limited to the order in which the acts of the method are recited, but the invention includes embodiments in which the order is so limited. Furthermore, where the claims recite a composition, the invention encompasses methods of using the composition and methods of making the composition. Where the claims recite a composition, it should be understood that the invention encompasses methods of using the composition and methods of making the composition.

### REFERENCES

1. von Figura, K., Schmidt, B., Selmer, T., and Dierks, T. (1998) A novel protein modification generating an aldehyde group in sulfatases: its role in catalysis and disease. BioEssays 20, 505-510
2. Diez-Roux, G. and Ballabio, A. (2005) Sulfatases and human disease. Annu. Rev. Genomics Hum. Genet. 6, 355-379
3. Schmidt, B., Selmer, T., Ingendoh, A., and von Figura, K. (1995) A novel amino acid modification in sulfatases that is defective in multiple sulfatase deficiency. Cell 82, 271-278
4. Dierks, T., Schmidt, B., Borissenko, L. V., Peng, J., Preusser, A., Mariappan, M., and von Figura, K. (2003) Multiple sulfatase deficiency is caused by mutations in the gene encoding the human C(alpha)-formylglycine generating enzyme. Cell 113, 435-444
5. Cosma, M. P., Pepe, S., Annunziata, I., Trott, D. A., Parenti, G., and Ballabio, A. (2003) The multiple sulfatase deficiency gene encodes an essential and limiting factor for the activity of sulfatases. Cell 113, 445-456
6. Cosma, M. P., Pepe, S., Parenti, G., Settembre, C., Annunziata, I., Wade-Martins, R., Di Domenico, C., Di Natale, P., Mankad, A., Cox, B., Uziel, G., Mancini, G. M., Zammarchi, E., Donati, M. A., Kleijer, W. J., Filocamo, M., Carrozzo, R., Carella, M., and Ballabio, A. (2004) Molecular and functional analysis of SUMF1 mutations in multiple sulfatase deficiency. Hum. Mutat. 23, 576-581
7. Annunziata, I., Bouche, V., Lombardi, A., Settembre, C., and Ballabio, A. (2007) Multiple sulfatase deficiency is due to hypomorphic mutations of the SUMF1 gene. Hum. Mutat. 28, 928
8. Schlotawa, L., Steinfeld, R., von Figura, K., Dierks, T., and Gartner, J. (2008) Molecular analysis of SUMF1 mutations: stability and residual activity of mutant formylglycine-generating enzyme determine disease severity in multiple sulfatase deficiency. Hum. Mutat. 29, 205
9. Buono, M., Visigalli, I., Bergamasco, R., Biffi, A. and Cosma, M. P. (2010) Sulfatase modifying factor 1-mediated fibroblast growth factor signaling primes hematopoietic multilineage development. J Exp Med. 207, 1647-1660
10. Dierks, T., Dickmanns, A., Preusser-Kunze, A., Schmidt, B., Mariappan, M., von Figura, K., Ficner, R., and Rudolph, M. G. (2005) Molecular basis for multiple sulfatase deficiency and mechanism for formylglycine generation of the human formylglycine-generating enzyme. Cell 121, 541-552
11. Landgrebe J, Dierks T, Schmidt B, von Figura K. (2003) The human SUMF1 gene, required for posttranslational sulfatase modification, defines a new gene family which is conserved from pro- to eukaryotes. Gene 316, 47-56
12. Mariappan, M., Gande, S. L., Radhakrishnan, K., Schmidt, B., Dierks, T and von Figura, K (2008) The non-catalytic N-terminal extension of formylglycine-generating enzyme is required for its biological activity and retention in the endoplasmic reticulum. J. Biol. Chem. 283, 11556-11564
13. Mariappan, M., Radhakrishnan, K., Dierks, T., Schmidt, B., and von Figura, K. (2008) ERp44 mediates a thiol-independent retention of formylglycine-generating enzyme in the endoplasmic reticulum. J. Biol. Chem. 283, 6375-6383
14. Fraldi, A., Zito, E., Annunziata, F., Lombardi, A., Cozzolino, M., Monti, M., Spampanato, C., Ballabio, A., Pucci, P., Sitia, R., Cosma, M. P. (2008) Multistep, sequential control of the trafficking and function of the multiple sulfatase deficiency gene product, SUMF1 by PDI, ERGIC-53 and ERp44. Hum. Mol. Genet. 17, 2610-2621
15. Preusser-Kunze, A., Mariappan, M., Schmidt, B., Gande, S. L., Mutenda, K., Wenzel, D., von Figura, K., and Dierks, T. (2005) Molecular characterization of the human Calpha-formylglycine-generating enzyme. J. Biol. Chem. 280, 14900-14910
16. Zito, E., Buono, M., Pepe, S., Settembre, C., Annunziata, I., Surace, E. M., Dierks, T., Monti, M., Cozzolino, M., Pucci, P., Ballabio, A., and Cosma, M. P. (2007) Sulfatase modifying factor 1 trafficking through the cells: from endoplasmic reticulum to the endoplasmic reticulum. EMBO J. 26, 2443-2453
17. Seidah, N. G. (2011) What lies ahead for the proproteinconvertases?. Ann. N. Y. Acad. Sci. 1220, 149-161
18. Khatib, A. M.; Siegfried, G.; Prat, A.; Luis, J.; Chrétien, M.; Metrakos, P.; Seidah, N. G. (2001) Inhibition of proproteinconvertases is associated with loss of growth and tumorigenicity of HT-29 human colon carcinoma cells: importance of insulin-like growth factor-1 (IGF-1) receptor processing in IGF-1-mediated functions. J. Biol. Chem. 276, 30686-30693
19. Schneider T. D., Stephens R. M. (1990) Sequence logos: a new way to display consensus sequences. Nucleic Acids Res. 18, 6097-6100
20. Crooks G. E., Hon G., Chandonia J. M., Brenner S. E. (2004) WebLogo: A sequence logo generator. Genome Res. 14, 1188-1190
21. Gordon, V. M., Klimpel, K. R., Arora, N., Henderson, M. A., and Leppla, S. H. (1995) Proteolytic activation of bacterial toxins by eukaryotic cells is performed by furin and by additional cellular proteases. Infect. Immun. 63, 82-87
22. Gieselmann, V., Schmidt, B., and Figura, K. von. 1992. In vitro mutagenesis of potential N-glycosylation sites of arylsulfatase A. Effects on glycosylation, phosphorylation, and intracellular sorting. J. Biol. Chem. 267, 13262-13266
23. Mariappan, M., Preusser-Kunze, A., Balleininger, M., Eiselt, N., Schmidt, B., Gande, S. L., Wenzel, D., Dierks, T., and von Figura, K. (2005) Expression, localization, structural, and functional characterization of pFGE, the paralog of the Calpha-formylglycine-generating enzyme. J. Biol. Chem. 280, 15173-15179
24. Duckert, P., Brunak, S., and Blom, N. (2004) Prediction of proproteinconvertase cleavage sites. Protein Eng. Des. Sel. 17, 107-112
25. Tian, S., Huajun, W., Wu, J. (2012) Computational prediction of furin cleavage sites by a hybrid method and understanding mechanism underlying diseases. Sci Rep. 2, 261
26. Dierks, T., Schlotawa, L., Frese, M. A., Radhakrishnan, K., von Figura, K., Schmidt, B (2009) Molecular basis of multiple sulfatase deficiency, mucolipidosis II/III and Niemann-Pick C1 disease—Lysosomal storage disorders caused by defects of non-lysosomal proteins. Biochim. Biophys. Acta 1793, 710-725
27. Erwin D. H., Laflamme M., Tweedt S. M., Sperling E. A., Pisani D., Peterson K. J. (2011) The Cambrian conundrum: early divergence and later ecological success in the early history of animals. Science 334, 1091-1097
28. Hosaka, M., Nagahama, M., Kim, W. S., Watanabe, T., Hatsuzawa, K., Ikemizu, J., Murakami, K., Nakayama, K. (1991) Arg-X-Lys/Arg-Arg motif as a signal for precursor cleavage catalyzed by furin within the constitutive secretory pathway. J. Biol. Chem. 266, 12127-12130
29. Remacle, A. G., Shiryaev, S. A., Oh, E. S., Cieplak, P., Srinivasan, A., Wei, G., Liddington, R. C., Ratnikov, B. I., Parent, A., Desjardins, R., Day, R., Smith, J. W., Lebl, M., Strongin, A. Y. (2008) Substrate cleavage analysis of furin and related proproteinconvertases A comparative study. J. Biol. Chem. 283, 20897-20906
30. Molloy, S. S., Bresnahan, P. A., Leppla, S. H., Klimpel, K. R., Thomas, G. (1992) Human furin is a calcium-dependent serine endoprotease that recognizes the sequence Arg-X-X-Arg and efficiently cleaves anthrax toxin protective antigen. J. Biol. Chem. 267, 16396-16402
31. Plaimauer, B., Mohr, G., Wernhart, W., Himmelspach, M., Dorner, F., Schlokat, U. (2001) ‘Shed’ furin: mapping of the cleavage determinants and identification of its C-terminus Biochem. J. 354, 689-695
32. Fey, J., Balleininger, M., Borissenko, L. V., Schmidt, B., von Figura, K., Dierks, T. (2001) Characterization of posttranslational formylglycine formation by luminal components of the endoplasmic reticulum. J. Biol. Chem. 276, 47021-47028
33. Tian, S., Jianhua, W. (2010) Comparative study of the binding pockets of mammalian proproteinconvertases and its implications for the design of specific small molecule inhibitors. Int. J. Biol. Sci. 6, 89-95
34. Carlson, B. L., Ballister, E. R., Skordalakes, E., King, D. S., Breidenbach, M. A., Gilmore, S. A., Berger, J. M., Bertozzi, C. R. (2008) Function and structure of a prokaryotic formylglycine-generating enzyme. J. Biol. Chem. 283, 20117-20125
35. Wu, P., Shui, W., Carlson, B. L., Hu, N., Rabuka, D., Lee, J., Bertozzi, C. R. (2009) Site-specific chemical modification of recombinant proteins produced in mammalian cells by using the genetically encoded aldehyde tag. Proc. Natl. Acad. Sci. U.S.A. 106, 3000-3005
36. Essalmani, R., Susan-Resiga, D., Chamberland, A., Abifadel, M., Creemers, J. W., Boileau, C., Seidah, N. G. and Prat, A. (2011) In vivo evidence that furin from hepatocytes inactivates PCSK9. J. Biol. Chem. 286, 4257-4263
37. Alam, S. (2013). Conformational changes in formylglycine-generating enzyme during the catalytic cycle: Role of reducing agent and calcium. Ph.D thesis (Unpublished), Georg-August University of Gottingen.
38. Ennemann E C, Radhakrishnan K, Mariappan M, Wachs M, Pringle T H, Schmidt B, Dierks T. (2013) Proproteinconvertases process and thereby inactivate formylglycine-generating enzyme. J Biol Chem. 22; 288(8):5828-39
39. Mariappan, M., Gande, S. L., Radhakrishnan, K., Schmidt, B., Dierks, T., und von Figura, K. (2008). The non-catalytic N-terminal extension of formylglycine-generating enzyme is required for its biological activity and retention in the endoplasmic reticulum. J. Biol. Chem. 283, 17, 11556-11564.
40. Preusser-Kunze, A., Mariappan, M., Schmidt, B., Gande, S. L., Mutenda, K., Wenzel, D., Figura, K. von, und Dierks, T. (2005). Molecular characterization of the human Calpha-formylglycine generating enzyme. J. Biol. Chem. 280, 15, 14900-14910.

1. Process for producing eukaryotic Cα-formylglycine Generating Enzyme (FGE) or a functional variant thereof having Cα-formylglycine generating activity or a fragment thereof, comprising:
(i) culturing an insect cell containing an isolated polynucleotide encoding the eukaryotic FGE enzyme or a functional variant or a fragment thereof in a medium under conditions permitting the expression of FGE or functional variant or a fragment thereof;
(ii) obtaining the produced FGE polypeptide of step (i).

2. The process of claim 1, wherein for the production of eukaryotic full length (fl) FGE₍₃₄₋₃₇₄ₐₐ₎, the polynucleotide encoding an eukaryotic fl FGE variant or a fragment thereof comprises a furin cleavage motif in the N-terminal region compared to the human fl FGE wild type (SEQ ID NO:2) which is non-cleavable, wherein the amino acid numbering of the fl FGE variant or fragment thereof corresponds to human FGE amino acid (SEQ ID NO:2).

3. The process of claim 1, wherein, the insect cell stably express the isolated polynucleotide.

4. The process of claim 1, wherein process further comprises the following steps which are to be conducted prior to step (i) of claim 1:
(ia) infecting the cell with a recombinant baculovirus, wherein the virus containing an isolated polynucleotide encoding the eukaryotic FGE or a functional variant thereof or a fragment thereof;
(ib) producing an infected insect cell capable of expressing the eukaryotic FGE or the functional variant thereof or the fragment thereof.

5. The process of claim 2, wherein the furin cleavage motif having at least a core motif of the amino acid formula:
R-Y-S-R
corresponding to human FGE amino acid (SEQ ID NO:2) aa 69-72.

6. The process of claim 4, wherein the baculovirus is selected from the group consisting of Autographa californica multicapsid nucleo polyhedrovirus (AcMNPV) and Bombyx mori nuclear polyhedrovirus (BmNPV).

7. The process of claim 1, wherein the insect cell is selected from the group consisting of cells derived from Spodopterafrugiperda, Trichoplusiani, Plutellasylostella, Manducasextra and Mamestrabrassicae.

8. The process of claim 7, wherein the insect cell is selected from the group consisting of Schneider cells S2 and S3, SF9, SF21, High FiveCells (BTI-TN-5B1-4), D.Mel-2 cells KCl cells and Mimi Sf9 insect cells.

9. The process of claim 1, wherein the eukaryotic FGE species is selected from the group consisting of mammalian, human, fungus, algae and insect.

10. The process of claim 1, wherein the species is human.

11. The process of claim 1, wherein the produced FGE polypeptide is secreted into the medium.

12. A eukaryotic polypeptide comprising a eukaryotic Cα-formylglycine Generating Enzyme (FGE) or a functional variant thereof having Cα-formylglycine generating activity or a FGE fragment obtainable by the process of claim 1, wherein the obtained eukaryotic polypeptide exhibit insect-specific post-translational modifications.

13. (canceled)

14. The eukaryotic polypeptide of claim 12 having an FGE obtainable by:
(i) culturing an insect cell containing an isolated polynucleotide encoding a eukaryotic FGE enzyme or a functional variant or a fragment thereof in a medium under conditions permitting the expression of FGE or functional variant or a fragment thereof;
(ii) obtaining the produced FGE polypeptide of step (i).

15. A eukaryotic FGE polypeptide variant having Cα-formylglycine generating activity, wherein the variant comprises an amino acid sequence further comprising a furin core cleavage motif wherein the furin core cleavage motif includes a core motif of the amino acid formula R-Y-S-R corresponding to human FGE amino acid (SEQ ID NO:2) aa 69-72 and having at least one amino acid modification in the furin-cleavage motif.

16. The eukaryotic FGE polypeptide variant of claim 15, wherein the at least one amino acid modification provides a modified FGE selected from the group consisting of:
i) an FGE variant having a non-cleavable furin cleavage motif; and
ii) an FGE variant having an optimized furin cleavage motif,
wherein the at least one amino acid modifications is located in the furin core cleavage motif, and at least one amino acid residue is changed compared to a corresponding wild type.

17. The eukaryotic FGE polypeptide variant of claim 15, wherein the at least one amino acid modifications takes place in the extended furin-cleavage motif comprising:
Xₙ₋₆-R-Y-S-R-Xₙ₊₈,
corresponding to human FGE amino acid (SEQ ID NO: 2) aa 63-80, wherein
(iii) Xₙ₋₆ is SSAAAH in position 63 to 68,
(iv) Xₙ₊₈ is EANAPGPV in position 73 to 80, and
wherein at least one amino acid residue is changed compared to a corresponding wild type.

18. The eukaryotic FGE polypeptide of claim 15, wherein the polypeptide exhibits at least one characteristic of the group consisting of:
(a) is at least a 41 kDa+/−3 kDa protein (SDS-PAGE);
(b) has a 55 aa N-terminal extension compared to a prokaryotic FGE protein;
(c) exhibits in vitro formlyglycine generation activity;
(d) is stable during chromatographic purification process;
(e) exhibits the N-terminal sequence EAN (Glu-Ala-Asn);
(f) exhibits an amino acid sequence having 85% or more identity to human FGE amino acid sequence (SEQ ID NO: 2); and
(g) catalyzes thiol-to-aldehyde oxidation of cysteine residues in the presence of glutathione.

19. The eukaryotic FGE polypeptide of claim 15, wherein
i) the variant comprises at least one of the substitutions selected from the group consisting of SEQ ID NO:8, SEQ ID NO: 10, SEQ ID NO:12, SEQ ID NO: 14, SEQ ID NO: 16, SEQ ID NO: 18, SEQ ID NO:20; (SEQ ID NO:22, SEQ ID NO:24; SEQ ID NO:26, SEQ ID NO:4, SEQ ID NO:29 and SEQ ID NO: 31, and combinations thereof; and
ii) wherein the amino acid sequence of the variant comprises of an amino acid sequence having at least a degree of identity to SEQ ID NO: 2 of at least 60%.

20. An in vitro method of producing an aldehyde tag in a polypeptide of interest, comprising the steps of
(i) incubating a polypeptide having a motif comprising a sulfatase motif having a 2-formylglycine, together with the FGE polypeptide obtained by the process of claim 1 having the presence of a reducing agent under conditions suitable for enzymatic activity to allow conversion of an amino acid residue to a formylglycine (FGIy) residue in the polypeptide and producing a converted tagged polypeptide;
(ii) recovering the polypeptide with the newly generated tag.

21. The method of claim 20, further comprising the step of
(iii) attaching a moiety of interest to the newly generated tag, wherein the moiety is selected from the group consisting of detectable label, a small molecule, a peptide or a toxin.

22. The method of claim 20, wherein glutathione is used as a reducing agent.

23. The method of claim 21, wherein the polypeptide is a medicament or a vaccine.

24. The method of claim 20, wherein the produced polypeptide is a non-naturally occurring or modified non-naturally occurring, recombinant polypeptide.

25. The method of claim 24, wherein the modified non-naturally occurring, recombinant polypeptide comprising a heterologous sulfatase motif having a 2-formylglycine residue covalently attached to a moiety of interest.

26. The method of claim 25, wherein the modified non-naturally occurring, recombinant polypeptide is selected from the group consisting of an Fc fragment, an antibody, an antigen-binding fragment of an antibody, a blood factor, a fibroblast growth factor, a protein vaccine, and an enzyme.

27. A polypeptide with a tag obtained by the method of claim 20

28. A polypeptide with a tag obtained by the method of claim 21.


SEQUENCE LISTING (110 sequences)

SEQ ID NO: 1 - DNA, 1074 bp, Artificial Sequence
  matured /active full length (fl)  human FGE with 6x His tag
gcggatgaattcagccaggaggccgggaccggtgcgggcgcggggtcccttgcgggttcttgcggctgcggcacgccccagcggcctggcgcccatggcagttcggcagccgctcaccgatactcgcgggaggctaacgctccgggccccgtacccggagagcggcaactcgcgcactcaaagatggtccccatccctgctggagtatttacaatgggcacagatgatcctcagataaagcaggatggggaagcacctgcgaggagagttactattgatgccttttacatggatgcctatgaagtcagtaatactgaatttgagaagtttgtgaactcaactggctatttgacagaggctgagaagtttggcgactcctttgtctttgaaggcatgttgagtgagcaagtgaagaccaatattcaacaggcagttgcagctgctccctggtggttacctgtgaaaggcgctaactggagacacccagaagggcctgactctactattctgcacaggccggatcatccagttctccatgtgtcctggaatgatgcggttgcctactgcacttgggcagggaagcggctgcccacggaagctgagtgggaatacagctgtcgaggaggcctgcataatagacttttcccctggggcaacaaactgcagcccaaaggccagcattatgccaacatttggcagggcgagtttccggtgaccaacactggtgaggatggcttccaaggaactgcgcctgttgatgccttccctcccaatggttatggcttatacaacatagtggggaacgcatgggaatggacttcagactggtggactgttcatcattctgttgaagaaacgcttaacccaaaaggtcccccttctgggaaagaccgagtgaagaaaggtggatcctacatgtgccataggtcttattgttacaggtatcgctgtgctgctcggagccagaacacacctgatagctctgcttcgaatctgggattccgctgtgcagccgaccgcctgcccactatggacagcggccgcggaagccatcaccatcaccatcaccattaa

SEQ ID NO: 2 - protein, 357 aa, Artificial Sequence
  cloned matured acitve full length FGE with 6x His tag on C-terminus
ADEFSQEAGTGAGAGSLAGSCGCGTPQRPGAHGSSAAAHRYSREANAPGPVPGERQLAHSKMVPIPAGVFTMGTDDPQIKQDGEAPARRVTIDAFYMDAYEVSNTEFEKFVNSTGYLTEAEKFGDSFVFEGMLSEQVKTNIQQAVAAAPWWLPVKGANWRHPEGPDSTILHRPDHPVLHVSWNDAVAYCTWAGKRLPTEAEWEYSCRGGLHNRLFPWGNKLQPKGQHYANIWQGEFPVTNTGEDGFQGTAPVDAFPPNGYGLYNIVGNAWEWTSDWWTVHHSVEETLNPKGPPSGKDRVKKGGSYMCHRSYCYRYRCAARSQNTPDSSASNLGFRCAADRLPTMDSGRGSHHHHHHH

SEQ ID NO: 3 - DNA, 1074 bp, Artificial Sequence
  fl FGE variant, R69A / R72A
gcggatgaattcagccaggaggccgggaccggtgcgggcgcggggtcccttgcgggttcttgcggctgcggcacgccccagcggcctggcgcccatggcagttcggcagccgctcacgcatactcggcggaggctaacgctccgggccccgtacccggagagcggcaactcgcgcactcaaagatggtccccatccctgctggagtatttacaatgggcacagatgatcctcagataaagcaggatggggaagcacctgcgaggagagttactattgatgccttttacatggatgcctatgaagtcagtaatactgaatttgagaagtttgtgaactcaactggctatttgacagaggctgagaagtttggcgactcctttgtctttgaaggcatgttgagtgagcaagtgaagaccaatattcaacaggcagttgcagctgctccctggtggttacctgtgaaaggcgctaactggagacacccagaagggcctgactctactattctgcacaggccggatcatccagttctccatgtgtcctggaatgatgcggttgcctactgcacttgggcagggaagcggctgcccacggaagctgagtgggaatacagctgtcgaggaggcctgcataatagacttttcccctggggcaacaaactgcagcccaaaggccagcattatgccaacatttggcagggcgagtttccggtgaccaacactggtgaggatggcttccaaggaactgcgcctgttgatgccttccctcccaatggttatggcttatacaacatagtggggaacgcatgggaatggacttcagactggtggactgttcatcattctgttgaagaaacgcttaacccaaaaggtcccccttctgggaaagaccgagtgaagaaaggtggatcctacatgtgccataggtcttattgttacaggtatcgctgtgctgctcggagccagaacacacctgatagctctgcttcgaatctgggattccgctgtgcagccgaccgcctgcccactatggacagcggccgcggaagccatcaccatcaccatcaccattaa

SEQ ID NO: 4 - protein, 357 aa, Artificial Sequence
  double fl FGE variant R69A / R72A
ADEFSQEAGTGAGAGSLAGSCGCGTPQRPGAHGSSAAAHAYSAEANAPGPVPGERQLAHSKMVPIPAGVFTMGTDDPQIKQDGEAPARRVTIDAFYMDAYEVSNTEFEKFVNSTGYLTEAEKFGDSFVFEGMLSEQVKTNIQQAVAAAPWWLPVKGANWRHPEGPDSTILHRPDHPVLHVSWNDAVAYCTWAGKRLPTEAEWEYSCRGGLHNRLFPWGNKLQPKGQHYANIWQGEFPVTNTGEDGFQGTAPVDAFPPNGYGLYNIVGNAWEWTSDWWTVHHSVEETLNPKGPPSGKDRVKKGGSYMCHRSYCYRYRCAARSQNTPDSSASNLGFRCAADRLPTMDSGRGSHHHHHHH

SEQ ID NO: 5 - DNA, 972 bp, Artificial Sequence
  delta 72 FGE inactive FGE cleaved at aa 72 post translational
gcggatcttggatcctccatggaattcgaggctaacgctccgggccccgtacccggagagcggcaactcgcgcactcaaagatggtccccatccctgctggagtatttacaatgggcacagatgatcctcagataaagcaggatggggaagcacctgcgaggagagttactattgatgccttttacatggatgcctatgaagtcagtaatactgaatttgagaagtttgtgaactcaactggctatttgacagaggctgagaagtttggcgactcctttgtctttgaaggcatgttgagtgagcaagtgaagaccaatattcaacaggcagttgcagctgctccctggtggttacctgtgaaaggcgctaactggagacacccagaagggcctgactctactattctgcacaggccggatcatccagttctccatgtgtcctggaatgatgcggttgcctactgcacttgggcagggaagcggctgcccacggaagctgagtgggaatacagctgtcgaggaggcctgcataatagacttttcccctggggcaacaaactgcagcccaaaggccagcattatgccaacatttggcagggcgagtttccggtgaccaacactggtgaggatggcttccaaggaactgcgcctgttgatgccttccctcccaatggttatggcttatacaacatagtggggaacgcatgggaatggacttcagactggtggactgttcatcattctgttgaagaaacgcttaacccaaaaggtcccccttctgggaaagaccgagtgaagaaaggtggatcctacatgtgccataggtcttattgttacaggtatcgctgtgctgctcggagccagaacacacctgatagctctgcttcgaatctgggattccgctgtgcagccgaccgcctgcccactatggacagcggccgcggaagccatcaccatcaccatcaccattaa

SEQ ID NO: 6 - protein, 323 aa, Artificial Sequence
  cleaved FGE in position 72 , inacitve FGE
ADLGSSMEFEANAPGPVPGERQLAHSKMVPIPAGVFTMGTDDPQIKQDGEAPARRVTIDAFYMDAYEVSNTEFEKFVNSTGYLTEAEKFGDSFVFEGMLSEQVKTNIQQAVAAAPWWLPVKGANWRHPEGPDSTILHRPDHPVLHVSWNDAVAYCTWAGKRLPTEAEWEYSCRGGLHNRLFPWGNKLQPKGQHYANIWQGEFPVTNTGEDGFQGTAPVDAFPPNGYGLYNIVGNAWEWTSDWWTVHHSVEETLNPKGPPSGKDRVKKGGSYMCHRSYCYRYRCAARSQNTPDSSASNLGFRCAADRLPTMDSGRGSHHHHHHH

SEQ ID NO: 7 - DNA, 33 bp, Artificial Sequence
  primer
tcggcagccgctcacgcatactcgcgggaggct

SEQ ID NO: 8 - protein, 357 aa, Artificial Sequence; identical to SEQ ID NO: 4
  FGE fl variant R(Arg) to A (Ala) in position 69

SEQ ID NO: 9 - DNA, 33 bp, Artificial Sequence
  Primer for generating R 69 K FGE
tcggcagccgctcacaaatactcgcgggaggct

SEQ ID NO: 10 - protein, 357 aa, Artificial Sequence
  FGE (FL) variant R 69 K
ADEFSQEAGTGAGAGSLAGSCGCGTPQRPGAHGSSAAAHKYSREANAPGPVPGERQLAHSKMVPIPAGVFTMGTDDPQIKQDGEAPARRVTIDAFYMDAYEVSNTEFEKFVNSTGYLTEAEKFGDSFVFEGMLSEQVKTNIQQAVAAAPWWLPVKGANWRHPEGPDSTILHRPDHPVLHVSWNDAVAYCTWAGKRLPTEAEWEYSCRGGLHNRLFPWGNKLQPKGQHYANIWQGEFPVTNTGEDGFQGTAPVDAFPPNGYGLYNIVGNAWEWTSDWWTVHHSVEETLNPKGPPSGKDRVKKGGSYMCHRSYCYRYRCAARSQNTPDSSASNLGFRCAADRLPTMDSGRGSHHHHHHH

SEQ ID NO: 11 - DNA, 33 bp, Artificial Sequence
  primer
gcagccgctcaccgagcctcgcgggaggctaac

SEQ ID NO: 12 - protein, 357 aa, Artificial Sequence
  Y70A FGE variant
ADEFSQEAGTGAGAGSLAGSCGCGTPQRPGAHGSSAAAHRASREANAPGPVPGERQLAHSKMVPIPAGVFTMGTDDPQIKQDGEAPARRVTIDAFYMDAYEVSNTEFEKFVNSTGYLTEAEKFGDSFVFEGMLSEQVKTNIQQAVAAAPWWLPVKGANWRHPEGPDSTILHRPDHPVLHVSWNDAVAYCTWAGKRLPTEAEWEYSCRGGLHNRLFPWGNKLQPKGQHYANIWQGEFPVTNTGEDGFQGTAPVDAFPPNGYGLYNIVGNAWEWTSDWWTVHHSVEETLNPKGPPSGKDRVKKGGSYMCHRSYCYRYRCAARSQNTPDSSASNLGFRCAADRLPTMDSGRGSHHHHHHH

SEQ ID NO: 13 - DNA, 33 bp, Artificial Sequence
  primer
gcagccgctcaccgaaagtcgcgggaggctaac

SEQ ID NO: 14 - protein, 357 aa, Artificial Sequence
  Y70K FGE fl variant
ADEFSQEAGTGAGAGSLAGSCGCGTPQRPGAHGSSAAAHRKSREANAPGPVPGERQLAHSKMVPIPAGVFTMGTDDPQIKQDGEAPARRVTIDAFYMDAYEVSNTEFEKFVNSTGYLTEAEKFGDSFVFEGMLSEQVKTNIQQAVAAAPWWLPVKGANWRHPEGPDSTILHRPDHPVLHVSWNDAVAYCTWAGKRLPTEAEWEYSCRGGLHNRLFPWGNKLQPKGQHYANIWQGEFPVTNTGEDGFQGTAPVDAFPPNGYGLYNIVGNAWEWTSDWWTVHHSVEETLNPKGPPSGKDRVKKGGSYMCHRSYCYRYRCAARSQNTPDSSASNLGFRCAADRLPTMDSGRGSHHHHHHH

SEQ ID NO: 15 - DNA, 33 bp, Artificial Sequence
  primer
gcagccgctcaccgattctcgcgggaggctaac

SEQ ID NO: 16 - protein, 357 aa, Artificial Sequence
  FGE fl variant Y70F
ADEFSQEAGTGAGAGSLAGSCGCGTPQRPGAHGSSAAAHRFSREANAPGPVPGERQLAHSKMVPIPAGVFTMGTDDPQIKQDGEAPARRVTIDAFYMDAYEVSNTEFEKFVNSTGYLTEAEKFGDSFVFEGMLSEQVKTNIQQAVAAAPWWLPVKGANWRHPEGPDSTILHRPDHPVLHVSWNDAVAYCTWAGKRLPTEAEWEYSCRGGLHNRLFPWGNKLQPKGQHYANIWQGEFPVTNTGEDGFQGTAPVDAFPPNGYGLYNIVGNAWEWTSDWWTVHHSVEETLNPKGPPSGKDRVKKGGSYMCHRSYCYRYRCAARSQNTPDSSASNLGFRCAADRLPTMDSGRGSHHHHHHH

SEQ ID NO: 17 - DNA, 33 bp, Artificial Sequence
  primer
gcagccgctcaccgatcctcgcgggaggctaac

SEQ ID NO: 18 - protein, 357 aa, Artificial Sequence
  FGE fl variant Y70S
ADEFSQEAGTGAGAGSLAGSCGCGTPQRPGAHGSSAAAHRSSREANAPGPVPGERQLAHSKMVPIPAGVFTMGTDDPQIKQDGEAPARRVTIDAFYMDAYEVSNTEFEKFVNSTGYLTEAEKFGDSFVFEGMLSEQVKTNIQQAVAAAPWWLPVKGANWRHPEGPDSTILHRPDHPVLHVSWNDAVAYCTWAGKRLPTEAEWEYSCRGGLHNRLFPWGNKLQPKGQHYANIWQGEFPVTNTGEDGFQGTAPVDAFPPNGYGLYNIVGNAWEWTSDWWTVHHSVEETLNPKGPPSGKDRVKKGGSYMCHRSYCYRYRCAARSQNTPDSSASNLGFRCAADRLPTMDSGRGSHHHHHHH

SEQ ID NO: 19 - DNA, 33 bp, Artificial Sequence
  primer
gccgctcaccgatacgcgcgggaggctaacgct

SEQ ID NO: 20 - protein, 357 aa, Artificial Sequence
  FGE fl variant S71A
ADEFSQEAGTGAGAGSLAGSCGCGTPQRPGAHGSSAAAHRYAREANAPGPVPGERQLAHSKMVPIPAGVFTMGTDDPQIKQDGEAPARRVTIDAFYMDAYEVSNTEFEKFVNSTGYLTEAEKFGDSFVFEGMLSEQVKTNIQQAVAAAPWWLPVKGANWRHPEGPDSTILHRPDHPVLHVSWNDAVAYCTWAGKRLPTEAEWEYSCRGGLHNRLFPWGNKLQPKGQHYANIWQGEFPVTNTGEDGFQGTAPVDAFPPNGYGLYNIVGNAWEWTSDWWTVHHSVEETLNPKGPPSGKDRVKKGGSYMCHRSYCYRYRCAARSQNTPDSSASNLGFRCAADRLPTMDSGRGSHHHHHHH

SEQ ID NO: 21 - DNA, 33 bp, Artificial Sequence
  primer
gccgctcaccgatacaggcgggaggctaacgct

SEQ ID NO: 22 - protein, 357 aa, Artificial Sequence
  FGE variant S71R
ADEFSQEAGTGAGAGSLAGSCGCGTPQRPGAHGSSAAAHRYRREANAPGPVPGERQLAHSKMVPIPAGVFTMGTDDPQIKQDGEAPARRVTIDAFYMDAYEVSNTEFEKFVNSTGYLTEAEKFGDSFVFEGMLSEQVKTNIQQAVAAAPWWLPVKGANWRHPEGPDSTILHRPDHPVLHVSWNDAVAYCTWAGKRLPTEAEWEYSCRGGLHNRLFPWGNKLQPKGQHYANIWQGEFPVTNTGEDGFQGTAPVDAFPPNGYGLYNIVGNAWEWTSDWWTVHHSVEETLNPKGPPSGKDRVKKGGSYMCHRSYCYRYRCAARSQNTPDSSASNLGFRCAADRLPTMDSGRGSHHHHHHH

SEQ ID NO: 23 - DNA, 33 bp, Artificial Sequence
  primer
gctcaccgatactcgaaggaggctaacgctccg

SEQ ID NO: 24 - protein, 357 aa, Artificial Sequence
  FGE fl variant R72K
ADEFSQEAGTGAGAGSLAGSCGCGTPQRPGAHGSSAAAHRYSKEANAPGPVPGERQLAHSKMVPIPAGVFTMGTDDPQIKQDGEAPARRVTIDAFYMDAYEVSNTEFEKFVNSTGYLTEAEKFGDSFVFEGMLSEQVKTNIQQAVAAAPWWLPVKGANWRHPEGPDSTILHRPDHPVLHVSWNDAVAYCTWAGKRLPTEAEWEYSCRGGLHNRLFPWGNKLQPKGQHYANIWQGEFPVTNTGEDGFQGTAPVDAFPPNGYGLYNIVGNAWEWTSDWWTVHHSVEETLNPKGPPSGKDRVKKGGSYMCHRSYCYRYRCAARSQNTPDSSASNLGFRCAADRLPTMDSGRGSHHHHHHH

SEQ ID NO: 25 - DNA, 33 bp, Artificial Sequence
  primer
gctcaccgatactcggcggaggctaacgctccg

SEQ ID NO: 26 - protein, 357 aa, Artificial Sequence
  FGE R72A variant
ADEFSQEAGTGAGAGSLAGSCGCGTPQRPGAHGSSAAAHRYSAEANAPGPVPGERQLAHSKMVPIPAGVFTMGTDDPQIKQDGEAPARRVTIDAFYMDAYEVSNTEFEKFVNSTGYLTEAEKFGDSFVFEGMLSEQVKTNIQQAVAAAPWWLPVKGANWRHPEGPDSTILHRPDHPVLHVSWNDAVAYCTWAGKRLPTEAEWEYSCRGGLHNRLFPWGNKLQPKGQHYANIWQGEFPVTNTGEDGFQGTAPVDAFPPNGYGLYNIVGNAWEWTSDWWTVHHSVEETLNPKGPPSGKDRVKKGGSYMCHRSYCYRYRCAARSQNTPDSSASNLGFRCAADRLPTMDSGRGSHHHHHHH

SEQ ID NO: 27 - DNA, 33 bp, Artificial Sequence
  primer
gctcacgcatactcggcggaggctaacgctccg

SEQ ID NO: 28 - DNA, 33 bp, Artificial Sequence
  primer
gccgctcaccgagccaggcgggaggctaacgct

SEQ ID NO: 29 - protein, 357 aa, Artificial Sequence
  FGE variant Y70A / S71R
ADEFSQEAGTGAGAGSLAGSCGCGTPQRPGAHGSSAAAHRARREANAPGPVPGERQLAHSKMVPIPAGVFTMGTDDPQIKQDGEAPARRVTIDAFYMDAYEVSNTEFEKFVNSTGYLTEAEKFGDSFVFEGMLSEQVKTNIQQAVAAAPWWLPVKGANWRHPEGPDSTILHRPDHPVLHVSWNDAVAYCTWAGKRLPTEAEWEYSCRGGLHNRLFPWGNKLQPKGQHYANIWQGEFPVTNTGEDGFQGTAPVDAFPPNGYGLYNIVGNAWEWTSDWWTVHHSVEETLNPKGPPSGKDRVKKGGSYMCHRSYCYRYRCAARSQNTPDSSASNLGFRCAADRLPTMDSGRGSHHHHHHH

SEQ ID NO: 30 - DNA, 33 bp, Artificial Sequence
  primer
caccgatactcgcggccggctaacgctccgggc

SEQ ID NO: 31 - protein, 357 aa, Artificial Sequence
  FGE variant E73P
ADEFSQEAGTGAGAGSLAGSCGCGTPQRPGAHGSSAAAHRYSRPANAPGPVPGERQLAHSKMVPIPAGVFTMGTDDPQIKQDGEAPARRVTIDAFYMDAYEVSNTEFEKFVNSTGYLTEAEKFGDSFVFEGMLSEQVKTNIQQAVAAAPWWLPVKGANWRHPEGPDSTILHRPDHPVLHVSWNDAVAYCTWAGKRLPTEAEWEYSCRGGLHNRLFPWGNKLQPKGQHYANIWQGEFPVTNTGEDGFQGTAPVDAFPPNGYGLYNIVGNAWEWTSDWWTVHHSVEETLNPKGPPSGKDRVKKGGSYMCHRSYCYRYRCAARSQNTPDSSASNLGFRCAADRLPTMDSGRGSHHHHHHH

SEQ ID NO: 32 - DNA, 27 bp, Artificial Sequence
  primer
ccggaattcagccaggaggccgggacc

SEQ ID NO: 33 - DNA, 23 bp, Artificial Sequence
  primer
cggatatcatggagataattaaa

SEQ ID NO: 34 - DNA, 30 bp, Artificial Sequence
  primer
ccggaattcatccgccgcaaaggcagaatg

SEQ ID NO: 35 - DNA, 27 bp, Artificial Sequence
  primer
ccggaattcagccaggaggccgggacc

SEQ ID NO: 36 - DNA, 32 bp, Artificial Sequence
  primer
ataatgcggccgctgtccatagtgggcaggcg

SEQ ID NO: 37 - DNA, 21 bp, Artificial Sequence
  primer
ccggattattcataccgtccc

SEQ ID NO: 38 - DNA, 21 bp, Artificial Sequence
  primer
cgtgtcgggtttaacattacg

SEQ ID NO: 39 - DNA, 27 bp, Artificial Sequence
  primer
ccggaattcgaggctaacgctccgggc

SEQ ID NO: 40 - DNA, 32 bp, Artificial Sequence; identical to SEQ ID NO: 36
  primer

SEQ ID NO: 41 - DNA, 21 bp, Artificial Sequence
  primer
ccggattattcataccgtccc

SEQ ID NO: 42 - DNA, 21 bp, Artificial Sequence
  primer
cgtgtcgggtttaacattacg

SEQ ID NO: 43 - DNA, 18 bp, Artificial Sequence
  primer
gagtgagcaagtgaagac

SEQ ID NO: 44 - protein, 6 aa, Artificial Sequence
  furin like cleavage motif within eukaryote comparison
KXYSKX

SEQ ID NO: 45 - protein, 16 aa, Artificial Sequence
  furin cleavage motif
AAAHRYSREANAPGPV

SEQ ID NO: 46 - protein, 23 aa, Artificial Sequence
  peptide for Fgly acitivity FGE and delta FGE variants
MTDFYVPVSLCTPSRAALLTGRS

SEQ ID NO: 47 - protein, 6 aa, Artificial Sequence
  fornylglycine recognition motif for FGE
VCXPSR

SEQ ID NO: 48 - protein, 4 aa, Artificial Sequence
  human furin cleavage motif in FGE protein
RYSR

SEQ ID NO: 49 - protein, 4 aa, Artificial Sequence
  canonical furin recognition sequence
RXKR

SEQ ID NO: 50 - protein, 5 aa, Artificial Sequence
  furin cleavage motif in eukaryote species from northern pike (ray-finned fish) to human
KRYSK

SEQ ID NO: 51 - protein, 4 aa, Artificial Sequence
  furin cleavage like motif in group III, i.e. the later diverging eukaryotes, in the mountain pine beetle (dendroctonusponderosae)
KYKR

SEQ ID NO: 52 - protein, 16 aa, Mus musculus
  PEPTIDE (1)..(16): furin cleavage site
AAAQRYSREANAPGLT

SEQ ID NO: 53 - protein, 16 aa, Spermophilus tridecemlineatus
  PEPTIDE (1)..(16): furin cleavage site
SAAHRYSREANAPSAL

SEQ ID NO: 54 - protein, 16 aa, Diplodus bellottii
  PEPTIDE (1)..(16): furin cleavage site
AAAHRYSREANAPPGP

SEQ ID NO: 55 - protein, 16 aa, sus scrofa
  PEPTIDE (1)..(16): furin cleavage site
AAAQQYSREANAPGPV

SEQ ID NO: 56 - protein, 16 aa, erinaceus europae
  PEPTIDE (1)..(16): furin cleavage site
AAAHRYSWEANAPGPD

SEQ ID NO: 57 - protein, 16 aa, dasypus novemcinctus
  PEPTIDE (1)..(16): furin cleavage site
AAAHGYSREANAQGRA

SEQ ID NO: 58 - protein, 16 aa, monodelphis domestica
  PEPTIDE (1)..(16): furin cleavage site
SAAHRYSREANVAEPA

SEQ ID NO: 59 - protein, 16 aa, macropus eugenii
  PEPTIDE (1)..(16): furin cleavage motif
SAAHKYSREANVAERA

SEQ ID NO: 60 - protein, 16 aa, anolis carolinensis
  PEPTIDE (1)..(16): furin cleavage motif
VASRKYSREVHLPQQP

SEQ ID NO: 61 - protein, 16 aa, gallus gallus
  PEPTIDE (1)..(16): furin cleavage motif
VATVRYSAAANDGRSP

SEQ ID NO: 62 - protein, 16 aa, meleagris gallopavo
  PEPTIDE (1)..(16): furin cleavage motif
AAARRYSAVANGGRSS

SEQ ID NO: 63 - protein, 16 aa, alligator mississippiensis
  PEPTIDE (1)..(16): furin cleavage motif
AAVRRYSPEANAQRPG

SEQ ID NO: 64 - protein, 16 aa, python molurus
  PEPTIDE (1)..(16): furin cleavage motif
VAARKYSLDANVSQQP

SEQ ID NO: 65 - protein, 16 aa, ambystoma mexicanum
  PEPTIDE (1)..(16): furin cleavage motif
HRAARYSREANEPLKA

SEQ ID NO: 66 - protein, 16 aa, xenopus tropicalis
  PEPTIDE (1)..(16): furin cleavage motif
DSPHKYSREANEPEPA

SEQ ID NO: 67 - protein, 16 aa, xenopus laevis
  PEPTIDE (1)..(16): furin cleavage motif
ENSHKYSREANEPEPT

SEQ ID NO: 68 - protein, 16 aa, tetraodon nigroviridis
  PEPTIDE (1)..(16): furin cleavage motif
VDGAKYSRGASRRDQT

SEQ ID NO: 69 - protein, 16 aa, tetraodon nigroviridis
  PEPTIDE (1)..(16): furin cleavage motif
EPGPKYSRGANGRDED

SEQ ID NO: 70 - protein, 16 aa, danio rerio
  PEPTIDE (1)..(16): furin cleavage motif
DVNRIYSKTANEGPDD

SEQ ID NO: 71 - protein, 16 aa, oncorhynchus mykiss
  PEPTIDE (1)..(16): furin cleavage motif
KESSKYSKKSNERHTD

SEQ ID NO: 72 - protein, 16 aa, oryzias latipes
MTEPKYSSAGSKSNGG

SEQ ID NO: 73 - protein, 16 aa, ictalurus punctatus
  PEPTIDE (1)..(16): furin cleavage site
DEDGKYSERANKEFVG

SEQ ID NO: 74 - protein, 16 aa, ictalurus furcatus
  PEPTIDE (1)..(16): furin cleavage site
DEDGKYSERANKEFVG

SEQ ID NO: 75 - protein, 16 aa, gasterosteus aculeatus
  PEPTIDE (1)..(16): furin cleavage motif
EEGSKYSEGANGRFVQ

SEQ ID NO: 76 - protein, 16 aa, oreochromis niloticus
  PEPTIDE (1)..(16): furin cleavage site
LDEDKYSKDANDRTNQ

SEQ ID NO: 77 - protein, 16 aa, osmerus mordax
  PEPTIDE (1)..(16): furin cleavage sites
SHASKYLQTTNEKPTL

SEQ ID NO: 78 - protein, 16 aa, esox lucius
  PEPTIDE (1)..(16): furin cleavage sites
RESGIYSKTSNEKLTD

SEQ ID NO: 79 - protein, 16 aa, ciona intestinalis
  PEPTIDE (1)..(16): furin cleavage sites
EVAEEPDLPLQKVSSD

SEQ ID NO: 80 - protein, 16 aa, ciona savignyi
  PEPTIDE (1)..(16): furin cleavage site
EVEEEPDIPLPTIPTG

SEQ ID NO: 81 - protein, 16 aa, halocynthia roretzi
  PEPTIDE (1)..(16): furin cleavage site
MDQYEVTENAEQHLVE

SEQ ID NO: 82 - protein, 16 aa, oikopleura dioica
  PETIDE (1)..(16): furin cleavage site
AGTFTMGDNEELMPGD

SEQ ID NO: 83 - protein, 16 aa, branchiostoma floridae
  PEPTIDE (1)..(16): furin cleavage site
PVEGEGGAEAPEFDKD

SEQ ID NO: 84 - protein, 16 aa, strongylocentrotus purpuratus
  PEPTIDE (1)..(16): furin cleavage site
ALEEKYSREANDPIDH

SEQ ID NO: 85 - protein, 16 aa, paracentrotus lividus
  PEPTIDE (1)..(16): furin cleavage site
PLALKYSKEVNDATGS

SEQ ID NO: 86 - protein, 16 aa, saccoglossus kowalevskii
  PEPTIDE (1)..(16): furin cleavage site
KSGGEVNDHHGVEQHD

SEQ ID NO: 87 - protein, 16 aa, drosophila melanogaster
  PEPTIDE (1)..(16): furin cleavage site
SGQVCQQRAQGAHSHY

SEQ ID NO: 88 - protein, 16 aa, anopheles gambiae
  PEPTIDE (1)..(16): furin cleavage site
KERVIFPTDAAQHSPS

SEQ ID NO: 89 - protein, 16 aa, mayetiola destructor
  PEPTIDE (1)..(16): furin cleavage site
SNENKDDSSNEMCSNP

SEQ ID NO: 90 - protein, 16 aa, bombyx mori
  PEPTIDE (1)..(16): furin cleavage site
LYSGNNNEQCSVENIS

SEQ ID NO: 91 - protein, 16 aa, apis mellifera
  PEPTIDE (1)..(16): furin cleavage site
YKKEIQDSCLANDILH

SEQ ID NO: 92 - protein, 16 aa, camponotus floridanus
GYCVIDNSKFDAIDIN

SEQ ID NO: 93 - protein, 16 aa, acyrthosiphon pisum
  PEPTIDE (1)..(16): furin cleavage site
VCTSSATSNSERLDSE

SEQ ID NO: 94 - protein, 16 aa, rhodnius prolixus
  PEPTIDE (1)..(16): furin cleavage site
CIPSSFLDLLKQTREN

SEQ ID NO: 95 - protein, 16 aa, pediculus humanus
  PEPTDIE (1)..(16): furin cleavage site
KVNFKDDAIFEEQEIS

SEQ ID NO: 96 - protein, 16 aa, tribolium castaneum
  peptide (1)..(16): furin cleavage site
EPHNKYSKTFNEGGDS

SEQ ID NO: 97 - protein, 16 aa, dendroctonus ponderosae
  PEPTIDE (1)..(16): furin cleavage site
NPSQKYKRDLNENPAN

SEQ ID NO: 98 - protein, 16 aa, leptinotarsa decemlineata
  PEPTIDE (1)..(16): furin cleavage site
NPSHKYMKESNEETGN

SEQ ID NO: 99 - protein, 16 aa, eriocheir sinensis
  PEPTIDE (1)..(16): furin cleavage site
SPETSPVLENNEESPN

SEQ ID NO: 100 - protein, 16 aa, amblyomma variegatum
  PEPTIDE (1)..(16): furin cleavage site
GSTSDDDEESRVEVED

SEQ ID NO: 101 - protein, 16 aa, rhipicephalus microplus
  PEPTIDE (1)..(16): furin cleavage site
NHDEKARLDSENAALN

SEQ ID NO: 102 - protein, 16 aa, aplysia californica
  PEPTIDE (1)..(16): furin cleavage site
SQAPESSDPSGSVGVD

SEQ ID NO: 103 - protein, 16 aa, lottia gigantea
  PEPTIDE (1)..(16): furin cleavage site
KGSEQADDKSGMYHPQ

SEQ ID NO: 104 - protein, 16 aa, nematostella vectensis
  PEPTIDE (1)..(16): furin cleavage site
KKFVKYSKKANVDQDI

SEQ ID NO: 105 - protein, 16 aa, acropora millepora
  PEPTIDE (1)..(16): furin cleavage site
KITDRKNGEGKFNLKS

SEQ ID NO: 106 - protein, 16 aa, montastraea faveolata
  PEPTIDE (1)..(16): furin cleavage site
MTLNTGNTDGEIKLKS

SEQ ID NO: 107 - protein, 16 aa, pleurobrachia pileus
  PEPTIDE (1)..(16): furin cleavage site
KISDMKRNEQQSEHPN

SEQ ID NO: 108 - protein, 16 aa, amphimedon queenslandica
  PEPTIDE (1)..(16): furin cleavage site
SVEKEESEEPKAQAEE

SEQ ID NO: 109 - protein, 5 aa, Artificial Sequence
  synthetic generated motif recognized by the FGE protein or variant
GALNS

SEQ ID NO: 110 - protein, 4 aa, Artificial Sequence
  naturally occuring sulfatase moiety
ARSA